(19) United States
(12) Patent Application Publication
(10) Pub. No.: US 2013/0172553 A1
(43) Pub. Date: Jul. 4, 2013

(54) CHIRAL SYNTHESIS OF ISOXAZOLINES, ISOXAZOLINE COMPOUNDS, AND USES THEREOF

(71) Applicants: Thais Sielecki-Dzurdz, Kennett Square, PA (US); James Pruitt, Landenberg, PA (US)

(72) Inventors: Thais Sielecki-Dzurdz, Kennett Square, PA (US); James Pruitt, Landenberg, PA (US)

(73) Assignee: CPSI STOCKHOLDER TRUST, New York, NY (US)

(21) Appl. No.: 13/512,292

(22) PCT Filed: Nov. 26, 2010
(86) PCT No.: PCT/US2010/058135

§ 371 Date: Feb. 5, 2013

Related U.S. Application Data
(60) Provisional application No. 61/264,406, filed on Nov. 25, 2009.

(51) Int. Cl. C07D 261/04; C07D 413/06; C07D 495/04; C07D 413/04

(52) U.S. Cl. CPC C07D 261/04; C07D 413/04; C07D 413/06; C07D 495/04
USPC 544/137; 548/240; 546/209; 544/367; 546/168; 546/146

(57) ABSTRACT

Processes for the chiral syntheses of isoxazolines and intermediates are described. Compounds prepared thereby, and methods of using the compounds are also described.

### BACKGROUND

1. Field of the Application

The present application relates to new synthetic routes to isoxazolines and intermediates, and further uses thereof.

2. Discussion of the Background

Many isoxazoline compounds are known. Conventional synthetic routes to isoxazolines have been used. An example of such a conventional route, which relies on the synthesis of racemic isoxazoline followed by chiral HPLC, is shown below:

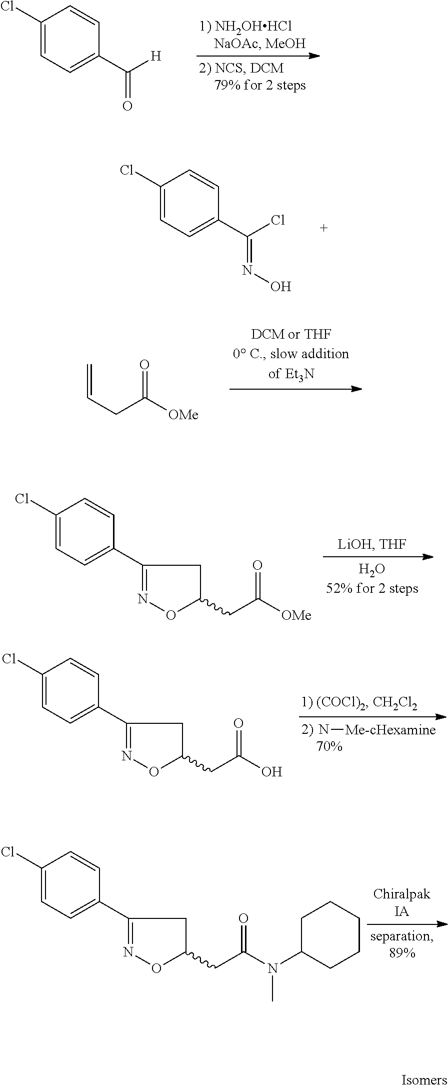

Another conventional synthetic route, which relies on the recrystallization of cinchonidine salt is shown below:

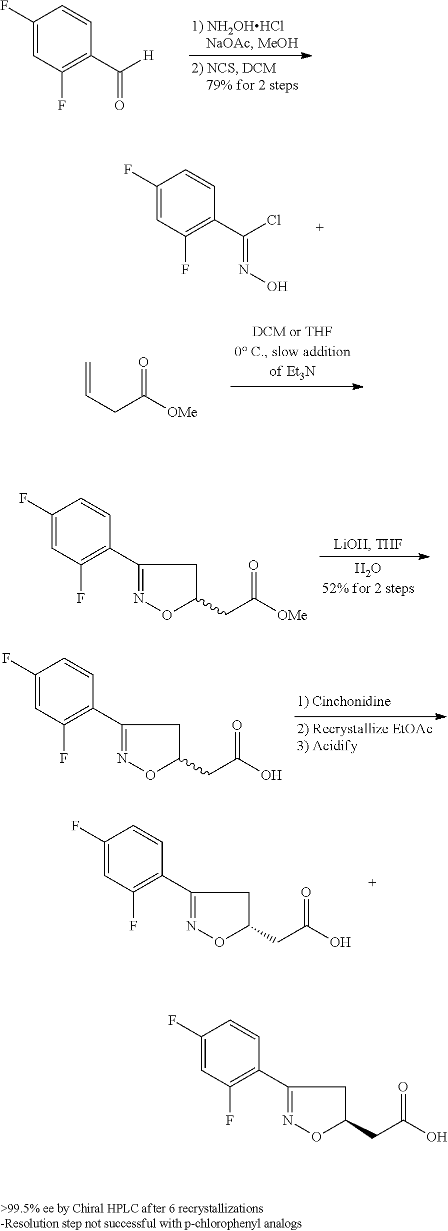

Neither of the conventional routes are completely satisfactory.

### DETAILED DESCRIPTION OF THE EMBODIMENTS

One embodiment provides a process, comprising:

reacting, in the presence of a base, a compound having the following formula (I):

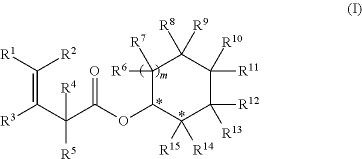

with a compound having the following formula (II):

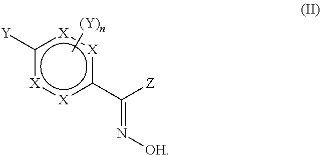

to produce a compound having the following formula (III):

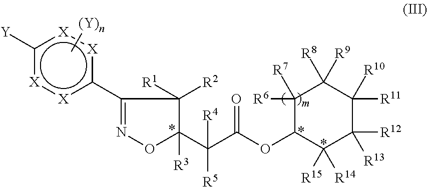

wherein at least the carbons marked “*” are chiral;

wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;

wherein R⁶, R⁷, R⁸, R⁹, R¹⁰, R¹¹, R¹², R¹³, R¹⁴, and R¹⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein m is 0 or 1; wherein any two of R⁶, R⁷, R⁸, R⁹, R¹⁰, R¹¹, R¹², R¹³, R¹⁴, and R¹⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; with the proviso that R¹⁴ and R¹⁵ are not identical groups;

wherein each X is independently carbon or nitrogen, wherein when any X is carbon, it comprises a Y substituent, n being an integer of from 1 to 4 and being the number of X's that are carbon;

wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms; and

wherein Z is iodine, bromine, chlorine, alkoxy group, aryloxy group, alkylcarbonyloxy group, alkoxycarbonyloxy group, alkylsulfonyloxy group, or alkoxysulfonyloxy group, arylcarbonyloxy group, aryloxycarbonyloxy group, arylsulfonyloxy group, or aryloxysulfonyloxy group.

In one embodiment, the compound having the formula (I) is selected from the following compounds:

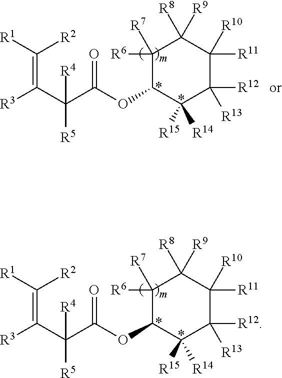

In one embodiment, the compound having the formula (III) is one or more selected from the following compounds:

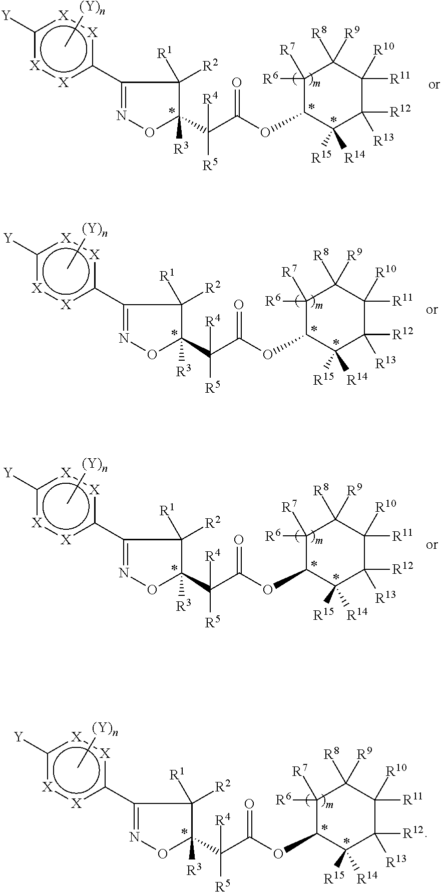

In one embodiment, the compound having the formula (I) is the following compound:

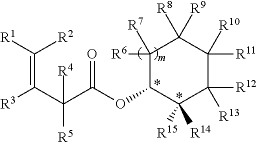

and the compound having the formula (III) is one or more of the following compounds:

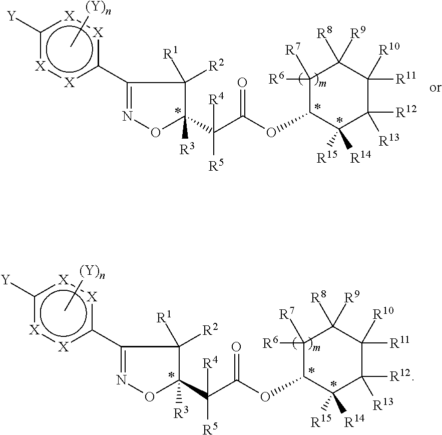

In one embodiment, the compounds having the following formulas may be separated from one another:

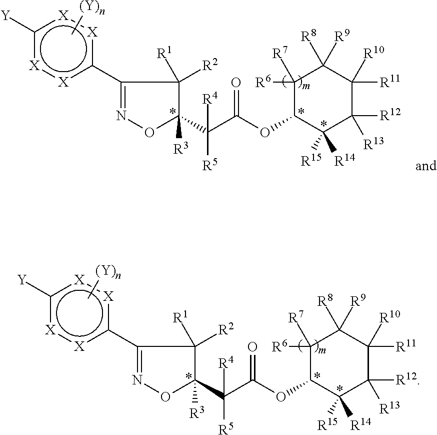

In one embodiment, the separating is carried out by a process comprising recrystallization, chromatography, or a combination thereof.

In one embodiment, the compound having the formula (I) is the following compound:

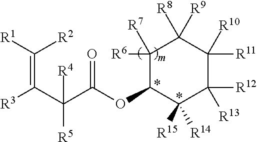

and the compound having the formula (III) is one or more of the following compounds:

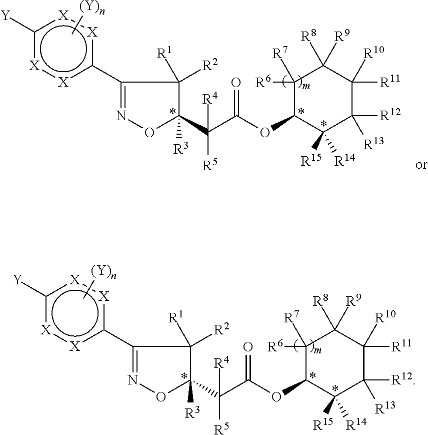

In one embodiment, the compounds having the formulas are separated from one another:

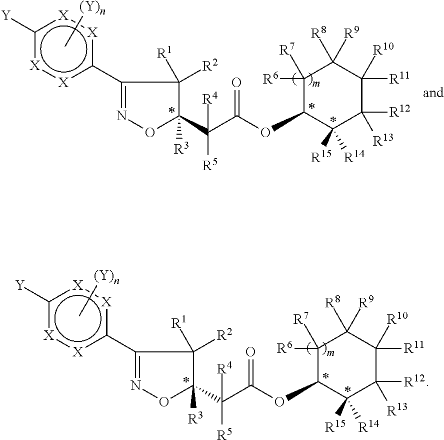

In one embodiment, the separating is carried out by a process comprising recrystallization, chromatography, or a combination thereof.

In one embodiment, the compound having the formula (III) is selected from one of the following compounds:

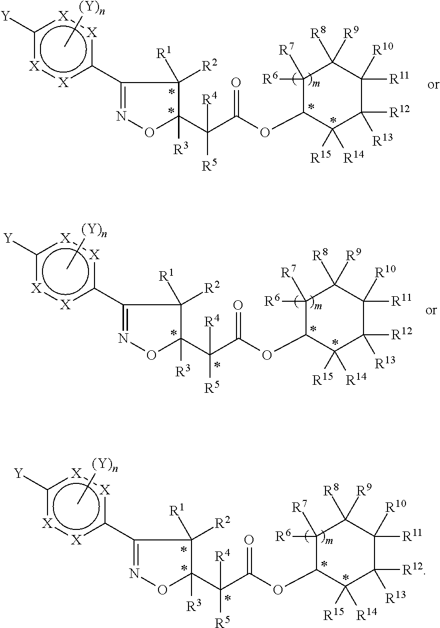

In one embodiment, R¹, R², R³, R⁴, and R⁵ are each independently hydrogen, an alkyl group, a cycloalkyl group, an alkenyl group, an alkynyl group, an oxo group, an aryl group, a heterocyclic group, a heteroaryl group, an aralkyl group, a heteroaralkyl group, an amino group, an alkylamino group, a dialkylamino group, an amidine group, an amide group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, R⁶, R⁷, R⁸, R⁹, R¹⁰, R¹¹, R¹², R¹³, R¹⁴, and R¹⁵ are each independently hydrogen, an alkyl group, a cycloalkyl group, a halo group, an alkenyl group, an alkynyl group, a hydroxy group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein m is 0 or 1; wherein any two of R⁶, R⁷, R⁸, R⁹, R¹⁰, R¹¹, R¹², R¹³, R¹⁴, and R¹⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; with the proviso that R¹⁴ and R¹⁵ are not identical groups.

In one embodiment, Z is iodine, bromine, or chlorine.

In one embodiment, one or both of R⁴ and R⁵ are hydrogen.

In one embodiment, only one of R⁴ and R⁵ is hydrogen.

In one embodiment, at least one of the compounds having formula (I), (III), or a combination thereof, in which one or both of R⁴ and R⁵ are hydrogen, is reacted with an electrophile in the presence of a base, to independently and optionally selectively replace one or both of the hydrogens at R⁴ and R⁵ with at least one selected from the group including an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, at least one of the compounds having formula (I), (III), or a combination thereof, in which only one of R⁴ and R⁵ is hydrogen, is contacted with a base in the absence of an electrophile, and allowed to equilibrate, to produce a thermodynamically favored equilibration product.

One embodiment provides a process, comprising:

reacting, in the presence of an aqueous bleach, a compound having the following formula (I):

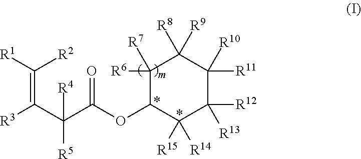

with a compound having the following formula (II-B):

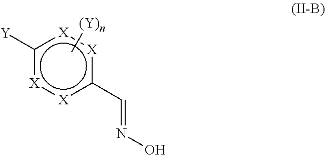

to produce a compound having the following formula (III):

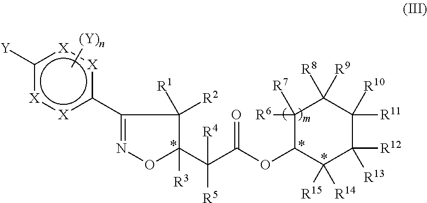

wherein at least the carbons marked “*” are chiral;

wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;

wherein R⁶, R⁷, R⁸, R⁹, R¹⁰, R¹¹, R¹², R¹³, R¹⁴, and R¹⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein m is 0 or 1; wherein any two of R⁶, R⁷, R⁸, R⁹, R¹⁰, R¹¹, R¹², R¹³, R¹⁴, and R¹⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; with the proviso that R¹⁴ and R¹⁵ are not identical groups;

wherein each X is independently carbon or nitrogen, wherein when any X is carbon, it comprises a Y substituent, n being an integer of from 1 to 4 and being the number of X's that are carbon;

and wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, the compound having the formula (I) is selected from the following compounds:

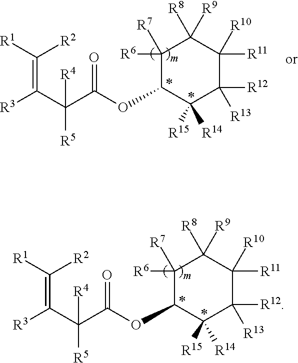

In one embodiment, the compound having the formula (III) is one or more selected from the following compounds:

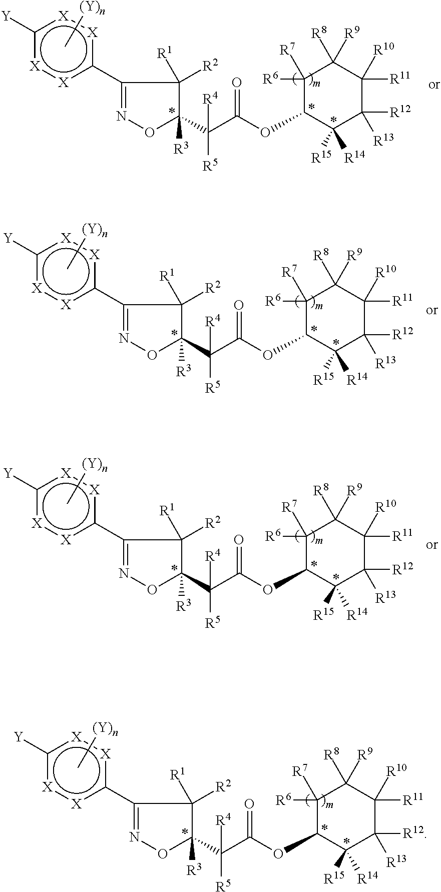

In one embodiment, the compound having the formula (I) is the following compound:

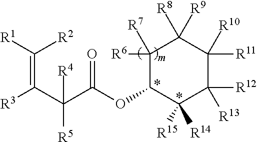

and the compound having the formula (III) is one or more of the following compounds:

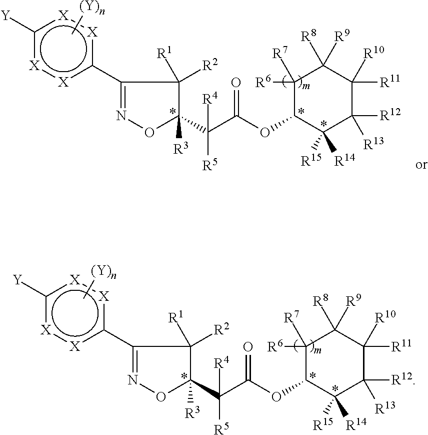

In one embodiment, the compounds having the following formulas are separated from one another:

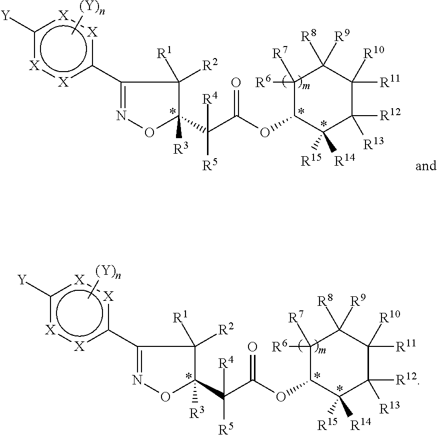

In one embodiment, the separating is carried out by a process comprising recrystallization, chromatography, or a combination thereof.

In one embodiment, the compound having the formula (I) is the following compound:

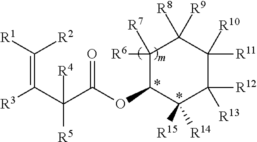

and the compound having the formula (III) is one or more of the following compounds:

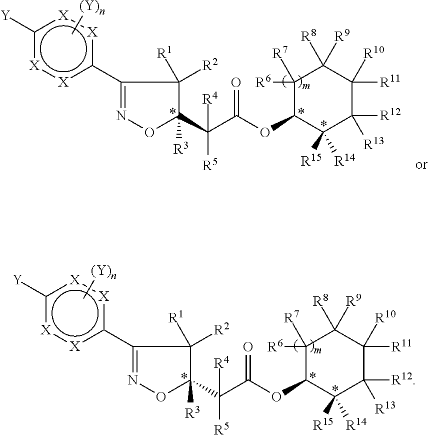

In one embodiment, the compounds having the following formulas are separated from one another:

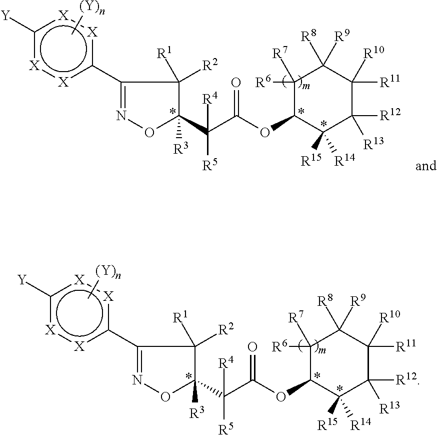

In one embodiment, the separating is carried out by a process comprising recrystallization, chromatography, or a combination thereof.

In one embodiment, the compound having the formula (III) is selected from one of the following compounds:

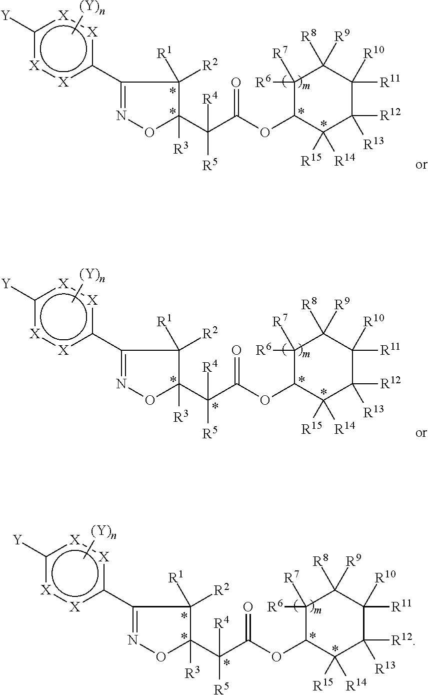

In one embodiment, R¹, R², R³, R⁴, and R⁵ are each independently hydrogen, an alkyl group, a cycloalkyl group, an alkenyl group, an alkynyl group, an oxo group, an aryl group, a heterocyclic group, a heteroaryl group, an aralkyl group, a heteroaralkyl group, an amino group, an alkylamino group, a dialkylamino group, an amidine group, an amide group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, R⁶, R⁷, R⁸, R⁹, R¹⁰, R¹¹, R¹², R¹³, R¹⁴, and R¹⁵ are each independently hydrogen, an alkyl group, a cycloalkyl group, a halo group, an alkenyl group, an alkynyl group, a hydroxy group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein m is 0 or 1; wherein any two of R⁶, R⁷, R⁸, R⁹, R¹⁰, R¹¹, R¹², R¹³, R¹⁴, and R¹⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; with the proviso that R¹⁴ and R¹⁵ are not identical groups.

In one embodiment, one or both of R⁴ and R⁵ are hydrogen.

In one embodiment, only one of R⁴ and R⁵ is hydrogen.

In one embodiment, at least one of the compounds having formula (I), (III), or a combination thereof, in which one or both of R⁴ and R⁵ are hydrogen, are reacted with an electrophile in the presence of a base, to independently and optionally selectively replace one or both of the hydrogens at R⁴ and R⁵ with at least one selected from the group including an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, at least one of the compounds having formula (I), (III), or a combination thereof, in which only one of R⁴ and R⁵ is hydrogen, is contacted with a base in the absence of an electrophile, and allowed to equilibrate, to produce a thermodynamically favored equilibration product.

One embodiment provides a process, comprising:

reacting a compound having the following formula (III):

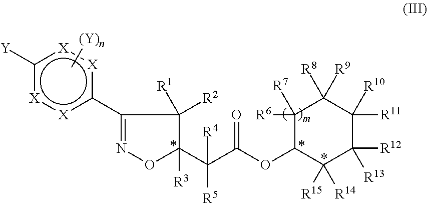

with an acid;

to produce a compound having the following formula (IV):

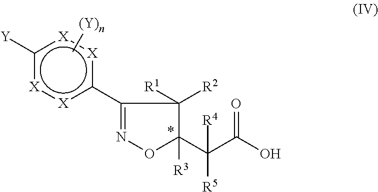

wherein at least the carbons marked “*” are chiral;

wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;

wherein R⁶, R⁷, R⁸, R⁹, R¹⁰, R¹¹, R¹², R¹³, R¹⁴, and R¹⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein m is 0 or 1; wherein any two of R⁶, R⁷, R⁸, R⁹, R¹⁰, R¹¹, R¹², R¹³, R¹⁴, and R¹⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; with the proviso that R¹⁴ and R¹⁵ are not identical groups;

wherein each X is independently carbon or nitrogen, wherein when any X is carbon, it comprises a Y substituent, n being an integer of from 1 to 4 and being the number of X's that are carbon; and

wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, the compound having the formula (III) is one or more selected from the following compounds:

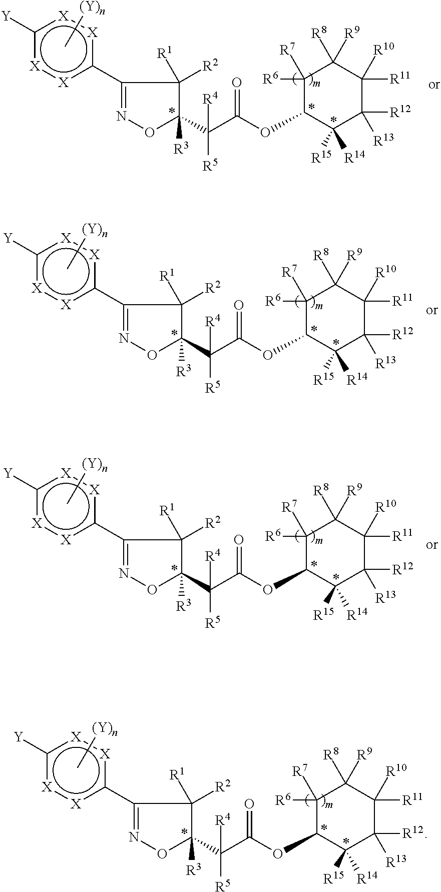

In one embodiment, the compound having the formula (IV) is selected from the following compounds:

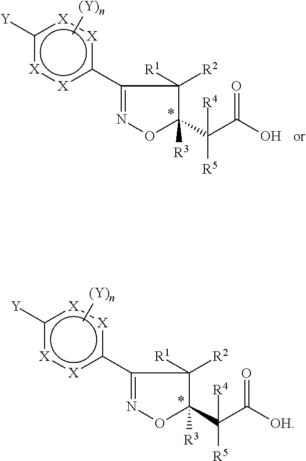

In one embodiment, the compound having the formula (III) is the following compound:

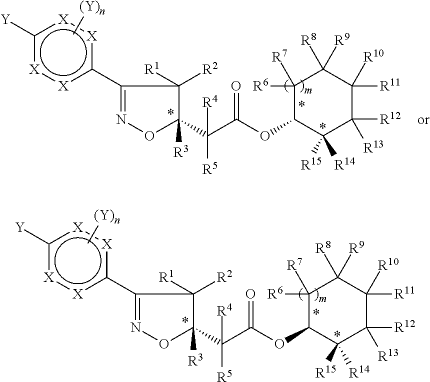

and the compound having the formula (IV) is the following compound:

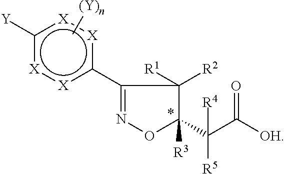

In one embodiment, the compound having the formula (III) is the following compound:

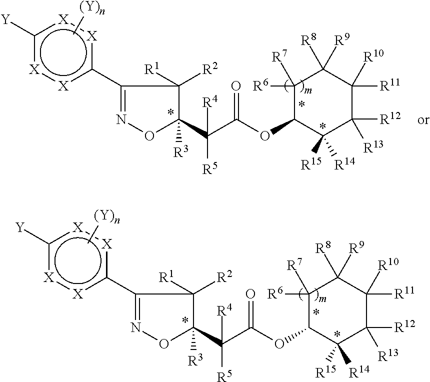

and the compound having the formula (IV) is the following compound:

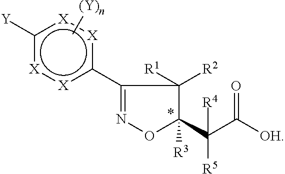

In one embodiment, R¹, R², R³, R⁴, and R⁵ are each independently hydrogen, an alkyl group, a cycloalkyl group, an alkenyl group, an alkynyl group, an oxo group, an aryl group, a heterocyclic group, a heteroaryl group, an aralkyl group, a heteroaralkyl group, an amino group, an alkylamino group, a dialkylamino group, an amidine group, an amide group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, R⁶, R⁷, R⁸, R⁹, R¹⁰, R¹¹, R¹², R¹³, R¹⁴, and R¹⁵ are each independently hydrogen, an alkyl group, a cycloalkyl group, a halo group, an alkenyl group, an alkynyl group, a hydroxy group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group; a perhaloaryl group, or a perhaloaralkyl group; wherein m is 0 or 1; wherein any two of R⁶, R⁷, R⁸, R⁹, R¹⁰, R¹¹, R¹², R¹³, R¹⁴, and R¹⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; with the proviso that R¹⁴ and R¹⁵ are not identical groups.

In one embodiment, one or both of R⁴ and R⁵ are hydrogen.

In one embodiment, only one of R⁴ and R⁵ is hydrogen.

In one embodiment, the compound having formula (III), in which one or both of R⁴ and R⁵ are hydrogen, is reacted with an electrophile in the presence of a base, to independently and optionally selectively replace one or both of the hydrogens at R⁴ and R⁵ with at least one selected from the group including an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, the compound having formula (III), in which only one of R⁴ and R⁵ is hydrogen, is contacted with a base in the absence of an electrophile, and allowed to equilibrate, to produce a thermodynamically favored equilibration product.

One embodiment provides a process, comprising:

reacting, in the presence of an acid halide and simultaneously or subsequently at least one selected from a base, an inorganic base, a trialkylamine, an aromatic nitrogen-containing heterocycle, or an organometallic compound, a compound having the following formula (IV):

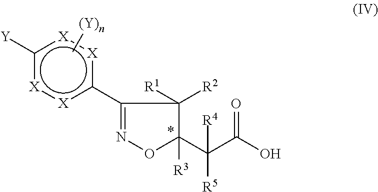

with a compound having at least one nucleophilic O, N, S, C, B or combination thereof, which forms a new bond with an electrophile;

to produce a compound having the following formula (V):

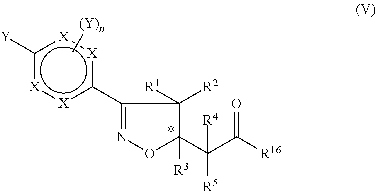

wherein at least the carbons marked “*” are chiral;

wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;

wherein each X is independently carbon or nitrogen, wherein when any X is carbon, it comprises a Y substituent, n being an integer of from 1 to 4 and being the number of X's that are carbon;

wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms;

and wherein R¹⁶ is an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein any two alkyl groups may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, the compound having the formula (IV) is selected from the following compounds:

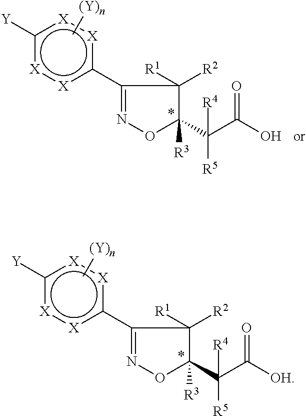

In one embodiment, the compound having the formula (V) is selected from the following compounds:

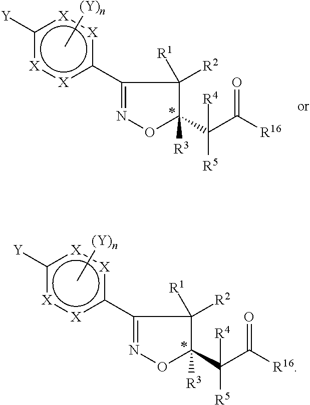

In one embodiment, the compound having the formula (IV) is the following compound:

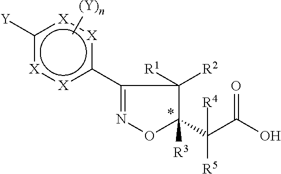

and the compound having the formula (V) is the following compound:

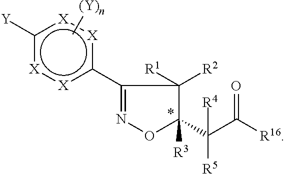

In one embodiment, the compound having the formula (IV) is the following compound:

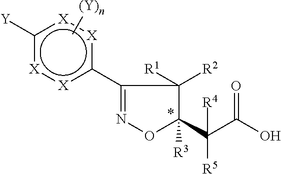

and the compound having the formula (V) is the following compound:

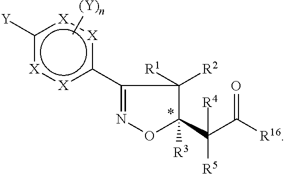

In one embodiment, R¹, R², R³, R⁴, and R⁵ are each independently hydrogen, an alkyl group, a cycloalkyl group, an alkenyl group, an alkynyl group, an oxo group, an aryl group, a heterocyclic group, a heteroaryl group, an aralkyl group, a heteroaralkyl group, an amino group, an alkylamino group, a dialkylamino group, an amidine group, an amide group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, one or both of R⁴ and R⁵ are hydrogen.

In one embodiment, only one of R⁴ and R⁵ is hydrogen.

In one embodiment, the compound having formula (V), in which one or both of R⁴ and R⁵ are hydrogen, is reacted with an electrophile in the presence of a base, to independently and optionally selectively replace one or both of the hydrogens at R⁴ and R⁵ with at least one selected from the group including an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, the compound having formula (V), in which only one of R⁴ and R⁵ is hydrogen, is contacted with a base in the absence of an electrophile, and allowed to equilibrate, to produce a thermodynamically favored equilibration product.

In one embodiment, at least one compound having formula (V), or salt thereof, or prodrug thereof, or combination thereof, is contacted with at least one pharmaceutically acceptable carrier, to produce a pharmaceutical composition.

One embodiment provides a process, comprising:

reacting, in the presence of an acid halide, a compound having the following formula (I-A):

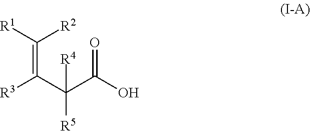

with a compound having the following formula (I-B):

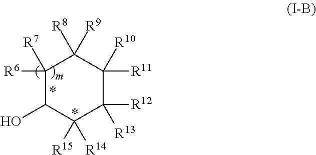

to produce a compound having the following formula (I):

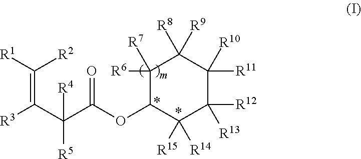

wherein at least the carbons marked “*” are chiral;

wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; and

wherein R⁶, R⁷, R⁸, R⁹, R¹⁰, R¹¹, R¹², R¹³, R¹⁴, and R¹⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein m is 0 or 1; wherein any two of R⁶, R⁷, R⁸, R⁹, R¹⁰, R¹¹, R¹², R¹³, R¹⁴, and R¹⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; with the proviso that R¹⁴ and R¹⁵ are not identical groups.

In one embodiment, the compound having the formula (I-B) is selected from the following compounds:

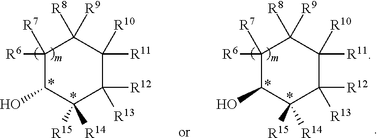

In one embodiment, the compound having the formula (I) is selected from the following compounds:

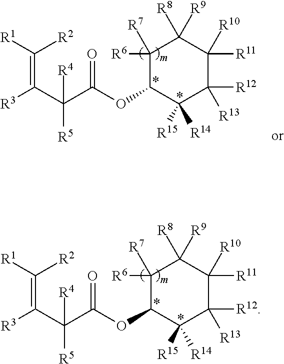

In one embodiment, the compound having the formula (I-B) is the following compound:

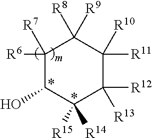

and the compound having the formula (I) is the following compound:

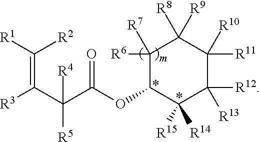

In one embodiment, the compound having the formula (I-B) is the following compound:

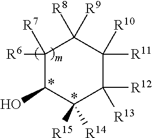

and the compound having the formula (I) is the following compound:

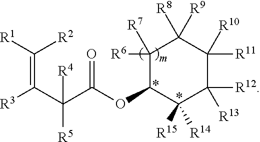

In one embodiment, R¹, R², R³, R⁴, and R⁵ are each independently hydrogen, an alkyl group, a cycloalkyl group, an alkenyl group, an alkynyl group, an oxo group, an aryl group, a heterocyclic group, a heteroaryl group, an aralkyl group, a heteroaralkyl group, an amino group, an alkylamino group, a dialkylamino group, an amidine group, an amide group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, R⁶, R⁷, R⁸, R⁹, R¹⁰, R¹¹, R¹², R¹³, R¹⁴, and R¹⁵ are each independently hydrogen, an alkyl group, a cycloalkyl group, a halo group, an alkenyl group, an alkynyl group, a hydroxy group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein m is 0 or 1; wherein any two of R⁶, R⁷, R⁸, R⁹, R¹⁰, R¹¹, R¹², R¹³, R¹⁴, and R¹⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; with the proviso that R¹⁴ and R¹⁵ are not identical groups.

In one embodiment, one or both of R⁴ and R⁵ are hydrogen.

In one embodiment, only one of R⁴ and R⁵ is hydrogen.

In one embodiment, the compound having formula (I), in which one or both of R⁴ and R⁵ are hydrogen, is reacted with an electrophile in the presence of a base, to independently and optionally selectively replace one or both of the hydrogens at R⁴ and R⁵ with at least one selected from the group including an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, the compound having formula (I), in which only one of R⁴ and R⁵ is hydrogen, is contacted with a base in the absence of an electrophile, and allowed to equilibrate, to produce a thermodynamically favored equilibration product.

One embodiment provides a process, comprising:

reacting a compound having the following formula (II-A):

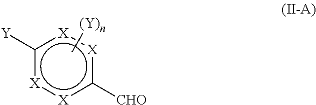

with a hydroxylamine salt and a base;

to produce a compound having the following formula (II-B):

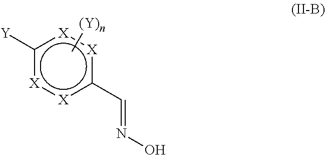

wherein each X is independently carbon or nitrogen, wherein when any X is carbon, it comprises a Y substituent, n being an integer of from 1 to 4 and being the number of X's that are carbon;

wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms.

One embodiment provides a process, comprising:

reacting a compound having the following formula (II-B):

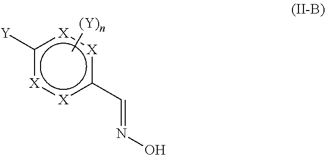

with a halogenating agent, optionally followed by at least one selected from a carboxylic acid or a sulfonic acid;

to produce a compound having the formula (II):

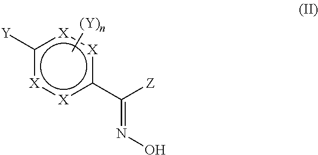

wherein each X is independently carbon or nitrogen, wherein when any X is carbon, it comprises a Y substituent, n being an integer of from 1 to 4 and being the number of X's that are carbon; and

wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms; and

wherein Z is iodine, bromine, chlorine, alkoxy group, aryloxy group, alkylcarbonyloxy group, alkoxycarbonyloxy group, alkylsulfonyloxy group, or alkoxysulfonyloxy group, arylcarbonyloxy group, aryloxycarbonyloxy group, arylsulfonyloxy group, or aryloxysulfonyloxy group.

In one embodiment, Z is iodine, bromine, or chlorine.

One embodiment provides a process, comprising:

reacting, in the presence of a base, a compound having the following formula (I):

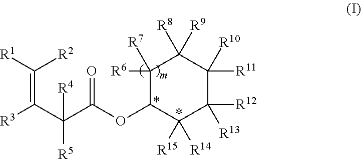

with a compound having the following formula (VI):

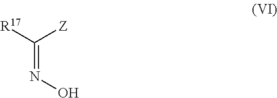

to produce a compound having the following formula (VII):

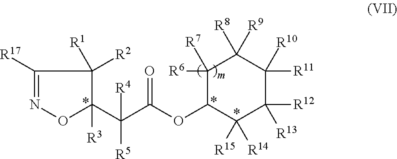

wherein at least the carbons marked “*” are chiral;

wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;

wherein R⁶, R⁷, R⁸, R⁹, R¹⁰, R¹¹, R¹², R¹³, R¹⁴, and R¹⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein m is 0 or 1; wherein any two of R⁶, R⁷, R⁸, R⁹, R¹⁰, R¹¹, R¹², R¹³, R¹⁴, and R¹⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; with the proviso that R¹⁴ and R¹⁵ are not identical groups;

wherein R¹⁷ is an alkyl group, a cycloalkyl group, an alkenyl group, an alkynyl group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms;

and wherein Z is iodine, bromine, chlorine, alkoxy group, aryloxy group, alkylcarbonyloxy group, alkoxycarbonyloxy group, alkylsulfonyloxy group, or alkoxysulfonyloxy group, arylcarbonyloxy group, aryloxycarbonyloxy group, arylsulfonyloxy group, or aryloxysulfonyloxy group.

In one embodiment, the compound having the formula (I) is selected from the following compounds:

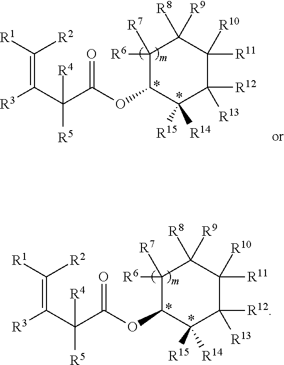

In one embodiment, the compound having the formula (VII) is selected from the following compounds:

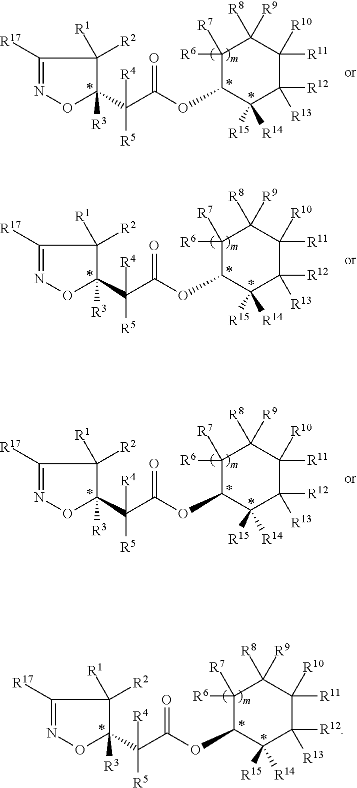

In one embodiment, the compound having the formula (I) is the following compound:

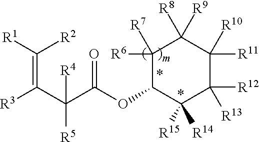

and the compound having the formula (VII) is one or more of the following compounds:

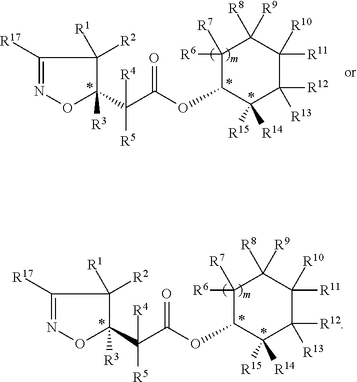

In one embodiment, the compounds having the following formulas are separated from one another:

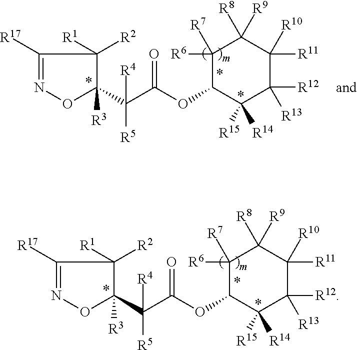

In one embodiment, the separating is carried out by a process comprising recrystallization, chromatography, or a combination thereof.

In one embodiment, the compound having the formula (I) is the following compound:

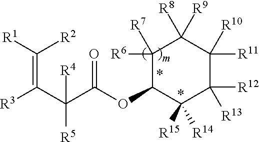

and the compound having the formula (VII) is one or more of the following compounds:

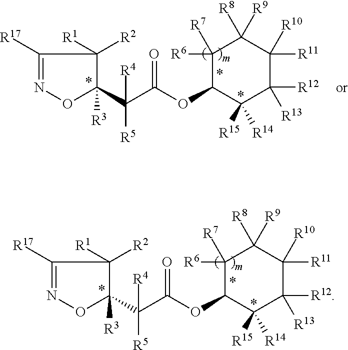

In one embodiment, the compounds having the following formulas are separated from one another:

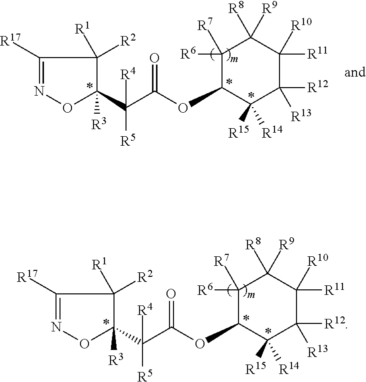

In one embodiment, the separating is carried out by a process comprising recrystallization, chromatography, or a combination thereof.

In one embodiment, the compound having the formula (VII) is selected from one of the following compounds:

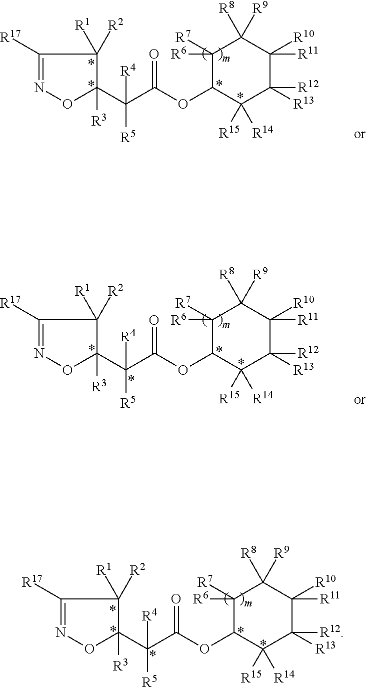

In one embodiment, R¹, R², R³, R⁴, and R⁵ are each independently hydrogen, an alkyl group, a cycloalkyl group, an alkenyl group, an alkynyl group, an oxo group, an aryl group, a heterocyclic group, a heteroaryl group, an aralkyl group, a heteroaralkyl group, an amino group, an alkylamino group, a dialkylamino group, an amidine group, an amide group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, R⁶, R⁷, R⁸, R⁹, R¹⁰, R¹¹, R¹², R¹³, R¹⁴, and R¹⁵ are each independently hydrogen, an alkyl group, a cycloalkyl group, a halo group, an alkenyl group, an alkynyl group, a hydroxy group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein m is 0 or 1; wherein any two of R⁶, R⁷, R⁸, R⁹, R¹⁰, R¹¹, R¹², R¹³, R¹⁴, and R¹⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; with the proviso that R¹⁴ and R¹⁵ are not identical groups.

In one embodiment, R¹⁷ is an alkyl group, a cycloalkyl group, an alkenyl group, an alkynyl group, an oxo group, an aryl group, a heterocyclic group, a heteroaryl group, an aralkyl group, a heteroaralkyl group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, Z is iodine, bromine, or chlorine.

In one embodiment, one or both of R⁴ and R⁵ are hydrogen.

In one embodiment, only one of R⁴ and R⁵ is hydrogen.

In one embodiment, at least one of the compounds having formula (I), (VII), or a combination thereof, in which one or both of R⁴ and R⁵ are hydrogen, is reacted with an electrophile in the presence of a base, to independently and optionally selectively replace one or both of the hydrogens at R⁴ and R⁵ with at least one selected from the group including an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, at least one of the compounds having formula (I), (VII), or a combination thereof, in which only one of R⁴ and R⁵ is hydrogen, is contacted with a base in the absence of an electrophile, and allowed to equilibrate, to produce a thermodynamically favored equilibration product.

One embodiment provides a process, comprising:

reacting, in the presence of an aqueous bleach, a compound having the following formula (I):

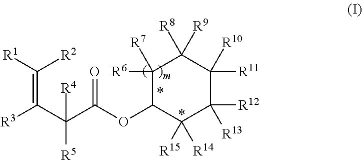

with a compound having the following formula (VI-B):

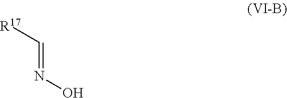

to produce a compound having the following formula (VII):

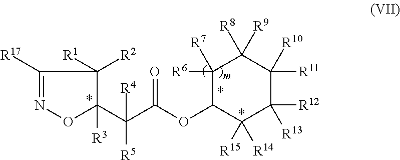

wherein at least the carbons marked “*” are chiral;

wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;

wherein R⁶, R⁷, R⁸, R⁹, R¹⁰, R¹¹, R¹², R¹³, R¹⁴, and R¹⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein m is 0 or 1; wherein any two of R⁶, R⁷, R⁸, R⁹, R¹⁰, R¹¹, R¹², R¹³, R¹⁴, and R¹⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; with the proviso that R¹⁴ and R¹⁵ are not identical groups;

and wherein R¹⁷ is an alkyl group, a cycloalkyl group, an alkenyl group, an alkynyl group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, the compound having the formula (I) is selected from the following compounds:

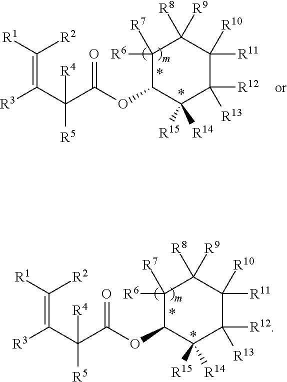

In one embodiment, the compound having the formula (VII) is one or more selected from the following compounds:

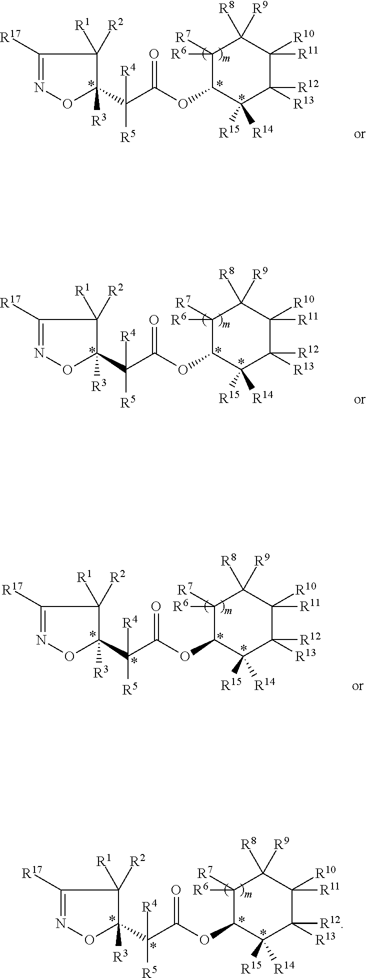

In one embodiment, the compound having the formula (I) is the following compound:

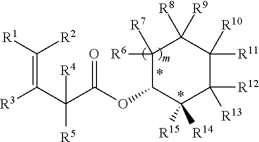

and the compound having the formula (VII) is one or more of the following compounds:

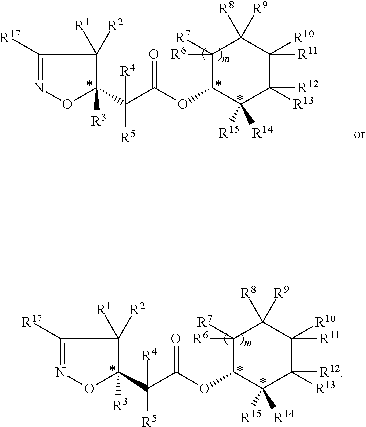

In one embodiment, the compounds having the following formulas are separated from one another:

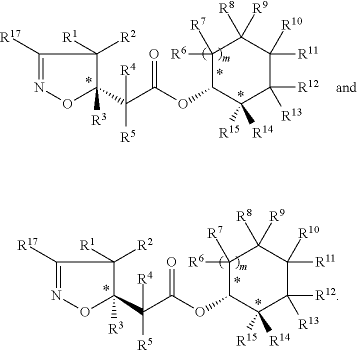

In one embodiment, the separating is carried out by a process comprising recrystallization, chromatography, or a combination thereof.

In one embodiment, the compound having the formula (I) is the following compound:

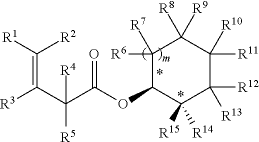

and the compound having the formula (VII) is one or more of the following compounds:

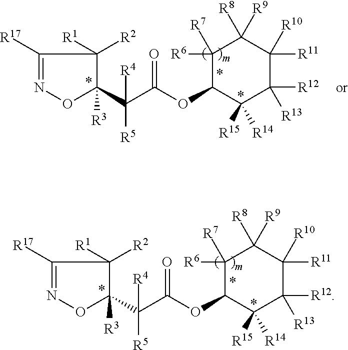

In one embodiment, the compounds having the following formulas are separated from one another:

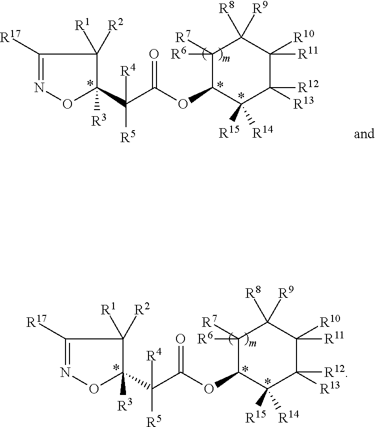

In one embodiment, the separating is carried out by a process comprising recrystallization, chromatography, or a combination thereof.

In one embodiment, the compound having the formula (VII) is selected from one of the following compounds:

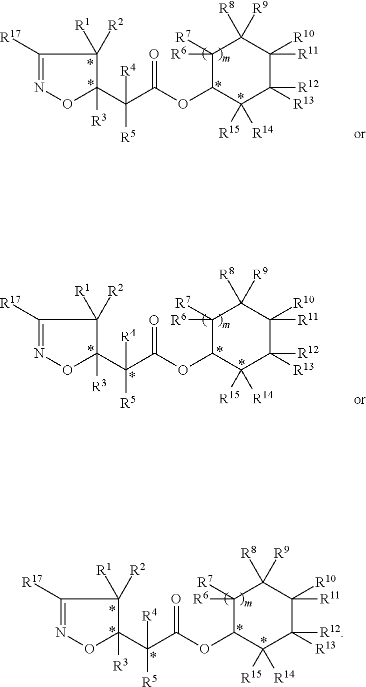

In one embodiment, R¹, R², R³, R⁴, and R⁵ are each independently hydrogen, an alkyl group, a cycloalkyl group, an alkenyl group, an alkynyl group, an oxo group, an aryl group, a heterocyclic group, a heteroaryl group, an aralkyl group, a heteroaralkyl group, an amino group, an alkylamino group, a dialkylamino group, an amidine group, an amide group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, R⁶, R⁷, R⁸, R⁹, R¹⁰, R¹¹, R¹², R¹³, R¹⁴, and R¹⁵ are each independently hydrogen, an alkyl group, a cycloalkyl group, a halo group, an alkenyl group, an alkynyl group, a hydroxy group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein m is 0 or 1; wherein any two of R⁶, R⁷, R⁸, R⁹, R¹⁰, R¹¹, R¹², R¹³, R¹⁴, and R¹⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; with the proviso that R¹⁴ and R¹⁵ are not identical groups.

In one embodiment, R¹⁷ is an alkyl group, a cycloalkyl group, an alkenyl group, an alkynyl group, an oxo group, an aryl group, a heterocyclic group, a heteroaryl group, an aralkyl group, a heteroaralkyl group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one in each group may be optionally and independently replaced with one or more independent heteroatoms.

In one embodiment, one or both of R⁴ and R⁵ are hydrogen.

In one embodiment, only one of R⁴ and R⁵ is hydrogen.

In one embodiment, at least one of the compounds having formula (I), (VII), or a combination thereof, in which one or both of R⁴ and R⁵ are hydrogen, is reacted with an electrophile in the presence of a base, to independently and optionally selectively replace one or both of the hydrogens at R⁴ and R⁵ with at least one selected from the group including an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, at least one of the compounds having formula (I), (VII), or a combination thereof, in which only one of R⁴ and R⁵ is hydrogen, is contacted with a base in the absence of an electrophile, and allowed to equilibrate, to produce a thermodynamically favored equilibration product.

One embodiment provides a process, comprising:

reacting a compound having the following formula (VII):

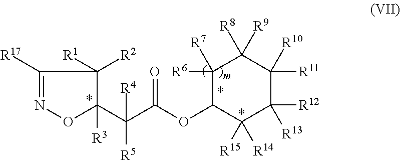

with an acid;

to produce a compound having the following formula (VIII):

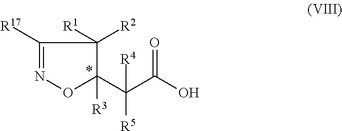

wherein at least the carbons marked “*” are chiral;

wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;

wherein R⁶, R⁷, R⁸, R⁹, R¹⁰, R¹¹, R¹², R¹³, R¹⁴, and R¹⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein m is 0 or 1; wherein any two of R⁶, R⁷, R⁸, R⁹, R¹⁰, R¹¹, R¹², R¹³, R¹⁴, and R¹⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; with the proviso that R¹⁴ and R¹⁵ are not identical groups;

and wherein R¹⁷ is an alkyl group, a cycloalkyl group, an alkenyl group, an alkynyl group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, the compound having the formula (VII) is one or more selected from the following compounds:

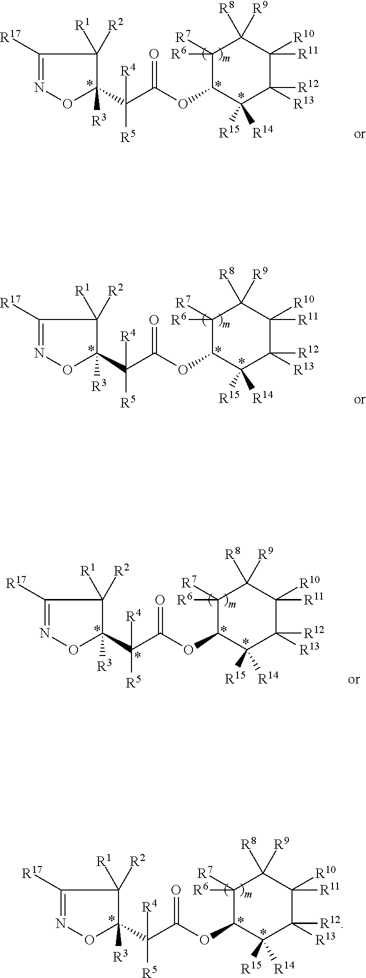

In one embodiment, the compound having the formula (VIII) is selected from the following compounds:

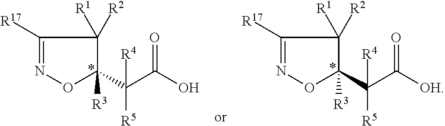

In one embodiment, the compound having the formula (VII) is one or more of the following compounds:

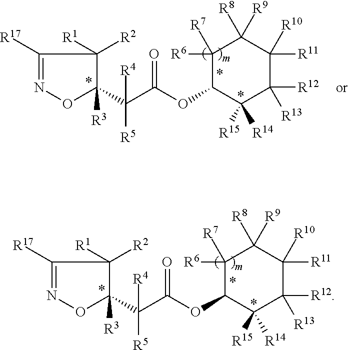

and the compound having the formula (VIII) is the following compound:

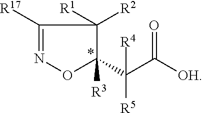

In one embodiment, the compound having the formula (VII) is one or more of the following compounds:

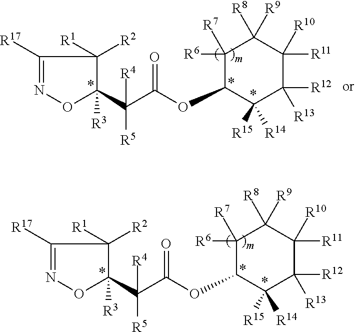

and the compound having the formula (VIII) is the following compound:

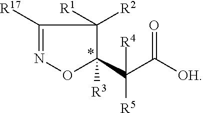

In one embodiment, R¹, R², R³, R⁴, and R⁵ are each independently hydrogen, an alkyl group, a cycloalkyl group, an alkenyl group, an alkynyl group, an oxo group, an aryl group, a heterocyclic group, a heteroaryl group, an aralkyl group, a heteroaralkyl group, an amino group, an alkylamino group, a dialkylamino group, an amidine group, an amide group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, R⁶, R⁷, R⁸, R⁹, R¹⁰, R¹¹, R², R¹³, R¹⁴, and R¹⁵ are each independently hydrogen, an alkyl group, a cycloalkyl group, a halo group, an alkenyl group, an alkynyl group, a hydroxy group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein m is 0 or 1; wherein any two of R⁶, R⁷, R⁸, R⁹, R¹⁰, R¹¹, R¹², R¹³, R¹⁴, and R¹⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; with the proviso that R¹⁴ and R¹⁵ are not identical groups.

In one embodiment, R¹⁷ is an alkyl group, a cycloalkyl group, an alkenyl group, an alkynyl group, an oxo group, an aryl group, a heterocyclic group, a heteroaryl group, an aralkyl group, a heteroaralkyl group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, one or both of R⁴ and R⁵ are hydrogen.

In one embodiment, only one of R⁴ and R⁵ is hydrogen.

In one embodiment, the compound having formula (VII), in which one or both of R⁴ and R⁵ are hydrogen, is reacted with an electrophile in the presence of a base, to independently and optionally selectively replace one or both of the hydrogens at R⁴ and R⁵ with at least one selected from the group including an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, the compound having formula (VII), in which only one of R⁴ and R⁵ is hydrogen, is contacted with a base in the absence of an electrophile, and allowed to equilibrate, to produce a thermodynamically favored equilibration product.

One embodiment provides a process, comprising:

reacting, in the presence of an acid halide and simultaneously or subsequently at least one selected from a base, an inorganic base, a trialkylamine, an aromatic nitrogen-containing heterocycle, or an organometallic compound, a compound having the following formula (VIII):

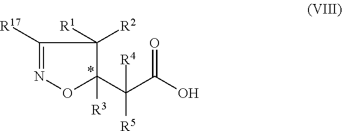

with a compound having at least one nucleophilic O, N, S, C, B or combination thereof, which forms a new bond with an electrophile;

to produce a compound having the following formula (IX):

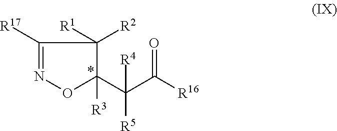

wherein at least the carbons marked “*” are chiral;

wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;

wherein R¹⁶ is an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein any two alkyl groups may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms

and wherein R¹⁷ is an alkyl group, a cycloalkyl group, an alkenyl group, an alkynyl group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, the compound having the formula (VIII) is selected from the following compounds:

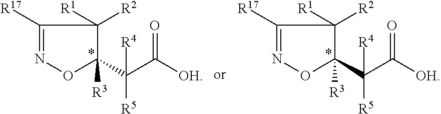

In one embodiment, the compound having the formula (IX) is selected from the following compounds:

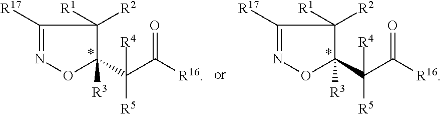

In one embodiment, the compound having the formula (VIII) is the following compound:

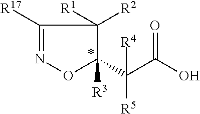

and the compound having the formula (IX) is the following compound:

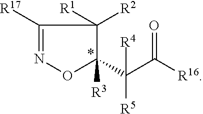

In one embodiment, the compound having the formula (VIII) is the following compound:

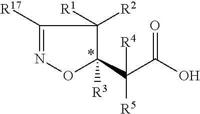

and the compound having the formula (IX) is the following compound:

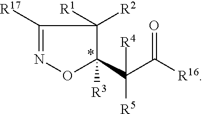

In one embodiment, R¹, R², R³, R⁴, and R⁵ are each independently hydrogen, an alkyl group, a cycloalkyl group, an alkenyl group, an alkynyl group, an oxo group, an aryl group, a heterocyclic group, a heteroaryl group, an aralkyl group, a heteroaralkyl group, an amino group, an alkylamino group, a dialkylamino group, an amidine group, an amide group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, R¹⁷ is an alkyl group, a cycloalkyl group, an alkenyl group, an alkynyl group, an oxo group, an aryl group, a heterocyclic group, a heteroaryl group, an aralkyl group, a heteroaralkyl group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, one or both of R⁴ and R⁵ are hydrogen.

In one embodiment, only one of R⁴ and R⁵ is hydrogen.

In one embodiment, the compound having formula (IX), in which one or both of R⁴ and R⁵ are hydrogen, is reacted with an electrophile in the presence of a base, to independently and optionally selectively replace one or both of the hydrogens at R⁴ and R⁵ with at least one selected from the group including an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, the compound having formula (IX), in which only one of R⁴ and R⁵ is hydrogen, is contacted with a base in the absence of an electrophile, and allowed to equilibrate, to produce a thermodynamically favored equilibration product.

In one embodiment, at least one compound having formula (IX), or salt thereof, or prodrug thereof, or combination thereof, is contacted with at least one pharmaceutically acceptable carrier, to produce a pharmaceutical composition.

One embodiment provides a process, comprising:

reacting a compound having the following formula (VI-A):

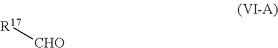

with a hydroxylamine salt and a base;

to produce a compound having the following formula (VI-B):

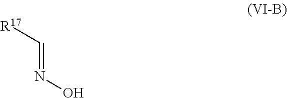

wherein R¹⁷ is an alkyl group, a cycloalkyl group, an alkenyl group, an alkynyl group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, R¹⁷ is an alkyl group, a cycloalkyl group, an alkenyl group, an alkynyl group, an oxo group, an aryl group, a heterocyclic group, a heteroaryl group, an aralkyl group, a heteroaralkyl group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

One embodiment provides a process, comprising:

reacting a compound having the following formula (VI-B):

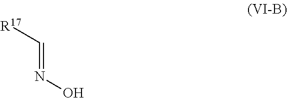

with a halogenating agent, optionally followed by at least one selected from a carboxylic acid or a sulfonic acid;

to produce a compound having the formula (VI):

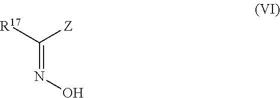

wherein R¹⁷ is an alkyl group, a cycloalkyl group, an alkenyl group, an alkynyl group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms;

and wherein Z is iodine, bromine, chlorine, alkoxy group, aryloxy group, alkylcarbonyloxy group, alkoxycarbonyloxy group, alkylsulfonyloxy group, or alkoxysulfonyloxy group, arylcarbonyloxy group, aryloxycarbonyloxy group, arylsulfonyloxy group, or aryloxysulfonyloxy group.

In one embodiment, R¹⁷ is an alkyl group, a cycloalkyl group, an alkenyl group, an alkynyl group, an oxo group, an aryl group, a heterocyclic group, a heteroaryl group, an aralkyl group, a heteroaralkyl group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

In one embodiment, Z is iodine, bromine, or chlorine.

In one embodiment, the compounds having formula (I):

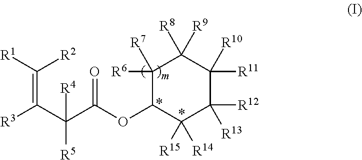

may be selected from compounds having the following structures:

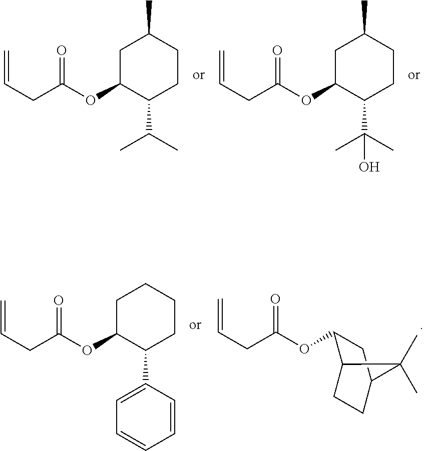

One embodiment relates to a composition, comprising one or more compounds having the following structures:

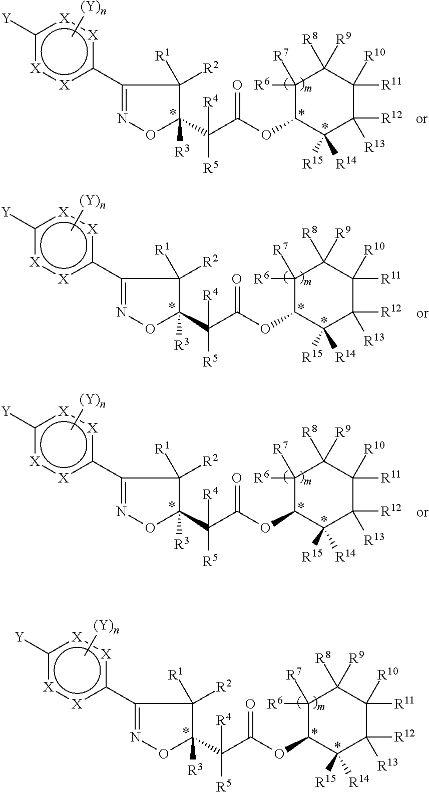

or combination thereof.

One embodiment relates to a composition, comprising one or more compounds having the following structures:

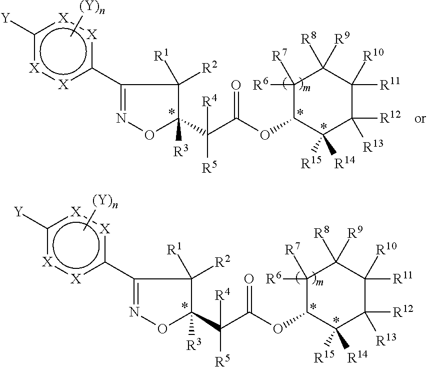

or a combination thereof.

One embodiment relates to a composition, comprising one or more compounds having the following structures:

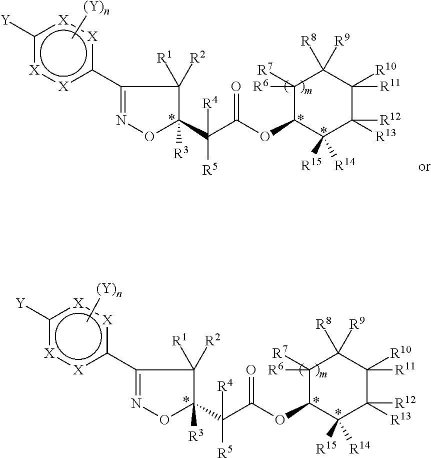

or combination thereof.

One embodiment relates to a composition, comprising one or more compounds having the following structures:

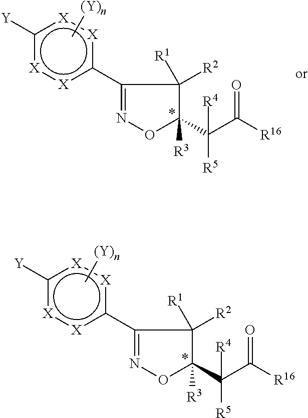

or a combination thereof.

One embodiment relates to a composition, comprising one or more compounds having the following structures:

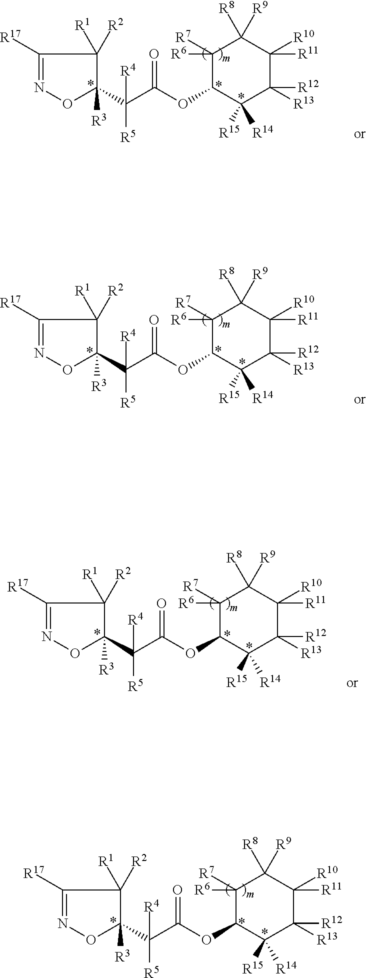

or combination thereof.

One embodiment relates to a composition, comprising one or more compounds having the following structures:

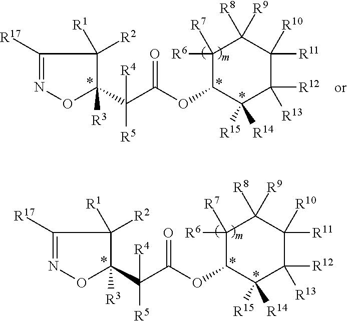

or combination thereof.

One embodiment relates to a composition, comprising one or more compounds having the following structures:

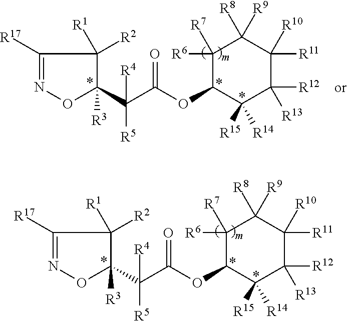

or combination thereof.

One embodiment relates to a composition, comprising one or more compounds having the following structures:

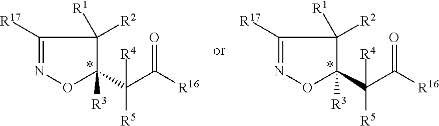

or combination thereof.

In one embodiment, at least one compound having formula (V), or salt thereof, or prodrug thereof, or combination thereof, may be considered pharmaceutically beneficial or have biological activity. In one embodiment, at least one compound having formula (V), or salt thereof, or prodrug thereof, or combination thereof, is in contact with at least one pharmaceutically acceptable carrier, in the form of a pharmaceutical composition.

In one embodiment, at least one compound having formula (IX), or salt thereof, or prodrug thereof, or combination thereof, may be considered pharmaceutically beneficial or have biological activity. In one embodiment, at least one compound having formula (V), or salt thereof, or prodrug thereof, or combination thereof, is in contact with at least one pharmaceutically acceptable carrier, in the form of a pharmaceutical composition.

In one embodiment, in the process, a compound having formula (II) is mixed with a base, the temperature of the resulting mixture is raised, and then slowly adding to this mixture a compound having formula (I). In one embodiment, a similar order of addition may be used for the reaction of the compounds having formulas (VI) and (I).

In one embodiment, a compound having formula (I) is mixed with base, the temperature of the resulting mixture is raised, and then slowly adding to this mixture a compound having formula (II). In one embodiment, a similar order of addition may be used for the reaction of the compounds having formulas (VI) and (I).

In one embodiment, the reaction temperature may suitably range from about −10° C. to about 200° C., which range includes all values and subranges therebetween. For example, the reaction temperature range may include −10, −9, −8, −7, −6, −5, −4, −3, −2, −1, 0, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200° C., or any combination thereof.

In one embodiment, the compound having formula (I) may be added at a rate of 0.005 mmol/hr to 100 mmol/hr, which range includes all values and subranges therebetween. For example, the range may include 0.005, 0.0075, 0.001, 0.002, 0.003, 0.004, 0.005, 0.006, 0.007, 0.008, 0.009, 0.01, 0.025, 0.05, 0.075, 0.1, 0.25, 0.5, 0.75, 1.0, 1.25, 1.5, 1.75, 2, 5, 10, 25, 50, 75, 100 mmol/hr, or any combination thereof.

In one embodiment, the solvent may be suitably chosen based on the solubility of the compound having formula (II). In one embodiment, the solvent may be suitably selected from organic solvents, dipolar aprotic solvents, nonpolar solvents. In one embodiment the solvent may be one or more of tetrahydrofuran, methyl ethyl ketone, ethyl acetate, acetonitrile, N,N-dimethylformamide, diemethyl sulfoxide, dichloromethane, hexane, benzene, diethyl ether, carbon tetrachloride, and the like, or combination thereof.

In one embodiment, the base may be added slowly. In one embodiment, excess base is avoided.

In one embodiment, in the reaction of compound (III) with an acid, to produce a compound of formula (IV), the solvent may be suitably chosen from dipolar aprotic solvent. Some examples of a dipolar aprotic solvent, which are not intended to be limiting, include tetrahydrofuran, methyl ethyl ketone, ethyl acetate, acetonitrile, N,N-dimethylformamide, diemethyl sulfoxide, and the like, or a combination thereof. In one embodiment, a similar solvent may be used in the reaction of compound (VII) with an acid to produce the compound of formula (VIII).

In one embodiment, in the reaction of compound (III) or (VII) with an acid, to produce, respectively, a compound of formula (IV) or (VIII), may be carried out under reflux (under seal or pressurized to avoid volatile acid loss); at reflux temperature of about 100° C.; over reaction time of about 3 days. Other temperatures and reaction times are possible, however. For example, the reflux temperature may be selected according to the solvent and in one embodiment may suitably range from 50° C. to 200° C., which range includes all values and subranges therebetween. In one embodiment, the reaction time may range from 2 hrs to 15 days, which range includes all values and subranges therebetween.

One embodiment relates to a process for producing compositions described herein, wherein one diastereomer is present in an amount greater than 50 mol % relative to other diastereomers present in the composition, if any.

One embodiment relates to a process for producing compositions described herein, in the absence of a purification step, in which one diastereomer is present in an amount greater than 50 mol % relative to other diastereomers present in the composition, if any. This range may suitably include greater than 50, 51, 52, 53, 54, 55, 56, 57, 58, 59, 60, 65, 70, 75, 80, 85, 90, 91, 92, 93, 94, 95, 96, 97, 98, 99, 99.0, 99.1, 99.2, 99.3, 99.4, 99.5, 99.6, 99.7, 99.8, 99.9, and 100 mol % of one diastereomer relative to any other diastereomer.

In one embodiment, a composition comprising diastereomers having formula (III) may be suitably produced by the present process. In one embodiment, in the resulting diastereomeric mixture, one diastereomer having formula (III) is present in an amount greater than 50 mol % relative to any other diastereomers having formula (III) that might be present. In one embodiment, even in the absence of a purification step, the process results in a diastereomeric mixture of compounds having formula (III) in which one diastereomer having formula (III) is present in an amount greater than 50 mol % relative to any other diastereomers having formula (III) that might be present. This range may suitably include greater than 50, 51, 52, 53, 54, 55, 56, 57, 58, 59, 60, 65, 70, 75, 80, 85, 90, 91, 92, 93, 94, 95, 96, 97, 98, 99, 99.0, 99.1, 99.2, 99.3, 99.4, 99.5, 99.6, 99.7, 99.8, 99.9, and 100 mol % of one diastereomer relative to any other diastereomer.

In one embodiment, a composition comprising diastereomers having formula (VII) may be suitably produced by the present process. In one embodiment, in the resulting diastereomeric mixture, one diastereomer having formula (VII) is present in an amount greater than 50 mol % relative to any other diastereomers having formula (VII) that might be present. In one embodiment, even in the absence of a purification step, the process results in a diastereomeric mixture of compounds having formula (VII) in which one diastereomer having formula (VII) is present in an amount greater than 50 mol % relative to any other diastereomers having formula (VII) that might be present. This range may suitably include greater than 50, 51, 52, 53, 54, 55, 56, 57, 58, 59, 60, 65, 70, 75, 80, 85, 90, 91, 92, 93, 94, 95, 96, 97, 98, 99, 99.0, 99.1, 99.2, 99.3, 99.4, 99.5, 99.6, 99.7, 99.8, 99.9, and 100 mol % of one diastereomer relative to any other diastereomer.

In one embodiment, the process results in an mixture of diastereomeric pairs of compounds having formula (III), in which the diastereomer:diastereomer molar ratio ranges from greater than 50:less than 50 to 100:0. This range of ratios includes all values and subranges therebetween, including greater than 50, 51, 52, 53, 54, 55, 56, 57, 58, 59, 60, 61, 62, 63, 64, 65, 66, 67, 68, 69, 70 71, 72, 73, 74, 75, 76, 77, 78, 79, 80, 81, 82, 83, 84, 85, 86, 87, 88, 89, 90, 91, 92, 93, 94, 95, 96, 97, 98, 99, 100: less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.

In one embodiment, the process results in an mixture of diastereomeric pairs of compounds having formula (VII), in which the diastereomer:diastereomer ratio ranges from greater than 50:less than 50 to 100:0. This range of ratios includes all values and subranges therebetween, including greater than 50, 51, 52, 53, 54, 55, 56, 57, 58, 59, 60, 61, 62, 63, 64, 65, 66, 67, 68, 69, 70 71, 72, 73, 74, 75, 76, 77, 78, 79, 80, 81, 82, 83, 84, 85, 86, 87, 88, 89, 90, 91, 92, 93, 94, 95, 96, 97, 98, 99, 100: less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.

In one embodiment, the composition comprising diastereomers having formula (III) may be reacted with or without further purification to produce compounds having formula (V) or a composition comprising enantiomers of compounds having formula (V). In one embodiment, in the resulting enantiomeric mixture, one enantiomer having formula (V) is present in an amount greater than 50 mol % relative to any other enantiomers having formula (V) that might be present. In one embodiment, even in the absence of a purification step, the process results in a enantiomeric mixture of compounds having formula (V) in which one enantiomer having formula (V) is present in an amount greater than 50 mol % relative to any other enantiomers having formula (V) that might be present. This range may suitably include greater than 50, 51, 52, 53, 54, 55, 56, 57, 58, 59, 60, 65, 70, 75, 80, 85, 90, 91, 92, 93, 94, 95, 96, 97, 98, 99, 99.0, 99.1, 99.2, 99.3, 99.4, 99.5, 99.6, 99.7, 99.8, 99.9, and 100 mol % of one enantiomer relative to any other enantiomer.

In one embodiment, the composition comprising diastereomers having formula (VII) may be reacted with or without further purification to produce compounds having formula (IX) or a composition comprising enantiomers of compounds having formula (IX). In one embodiment, in the resulting enantiomeric mixture, one enantiomer having formula (IX) is present in an amount greater than 50 mol % relative to any other enantiomers having formula (IX) that might be present. In one embodiment, even in the absence of a purification step, the process results in a enantiomeric mixture of compounds having formula (IX) in which one enantiomer having formula (IX) is present in an amount greater than 50 mol % relative to any other enantiomers having formula (IX) that might be present. This range may suitably include greater than 50, 51, 52, 53, 54, 55, 56, 57, 58, 59, 60, 65, 70, 75, 80, 85, 90, 91, 92, 93, 94, 95, 96, 97, 98, 99, 99.0, 99.1, 99.2, 99.3, 99.4, 99.5, 99.6, 99.7, 99.8, 99.9, and 100 mol % of one enantiomer relative to any other enantiomer.

In one embodiment, the mixtures of diastereomers, e.g., mixtures of diastereomers having formula (III) or mixtures of diasteromers having formula (VII) can be separated by taking advantage of their different physical properties, such as using either recrystallization or chromatography or a combination thereof. The recrystallizations can accomplished in organic solvents such as, but not limited to, pentane, hexane, cyclohexane, toluene, benzene, chlorobutane, dichloromethane, diethyl ether, tetrahydrofuran, dimethoxyethane, acetonitrile, methanol, ethanol or butanol or a combination of organic solvents with or without water. The chromatography can be accomplished with a silica gel or alumina solid phase, eluting with mixtures of organic solvents, with or without acidic or basic modifiers, such as triethylamine, aqueous ammonia, acetic acid or aqueous hydrochloric acid.

In one embodiment, an alkyl group is a univalent, acyclic, straight or branched, substituted or unsubstituted, saturated or unsaturated, hydrocarbon radical. In one embodiment, the alkyl group has the general formula (notwithstanding optional unsaturation, substitution or the like) —CₙH₂ₙ₊₁. In one embodiment, n is 1-20 ((C₁-C₂₀) alkyl), which may suitably include C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀ alkyl groups. In one embodiment, the alkyl group may be straight or branched, substituted or unsubstituted, saturated or unsaturated, or any combination thereof. In one embodiment, one or more hydrogens may be optionally and independently replaced by one or more substituent groups. In one embodiment, one or more carbon atoms may be optionally and independently replaced with one or more heteroatoms such as O, S, N, B, or any combination thereof. In one embodiment, the alkyl group may contain one or more double bond, one or more triple bond, or any combination thereof. In one embodiment, the alkyl group is attached to the parent structure through one or more independent divalent intervening substituent groups. Some examples of alkyl groups, which are not intended to be limiting, include methyl, ethyl, n-propyl, isopropyl, n-butyl, iso-butyl, secondary-butyl, tertiary-butyl, and the like.

In one embodiment, a cycloalkyl group is a univalent, mono- or polycyclic, substituted or unsubstituted, saturated or unsaturated hydrocarbon radical. In one embodiment, the cycloalkyl group has the general formula (notwithstanding optional unsaturation, substitution, or the like) —CₙH₂ₙ₋₁. In one embodiment, n is 3-20 ((C₃-C₂₀) cycloalkyl), which may suitably include C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀ cycloalkyl groups. In one embodiment, the cycloalkyl group is substituted or unsubstituted, saturated or unsaturated, mono-, bi-, tri-, or poly-cyclic, or any combination thereof. In one embodiment, one or more hydrogens may be optionally and independently replaced by one or more substituent groups. In one embodiment, the cycloalkyl group may have one or more sites of unsaturation, e.g., it may contain one or more double bond, one or more triple bond, or any combination thereof. In one embodiment, one or more carbon atoms may be optionally and independently replaced with one or more heteroatoms such as O, S, N, B, or any combination thereof. In one embodiment, the cycloalkyl group is attached to the parent structure through one or more independent divalent intervening substituent groups. Some examples of cycloalkyl groups, which are not intended to be limiting, include cyclopropyl, cyclobutyl, cyclopentyl, cyclohexyl, cycloheptyl, cyclooctyl, cyclononyl, cyclopentenyl, cyclohexenyl, bicyclo[2.2.1]heptanyl, bicyclo[3.2.1]octanyl and bicyclo[5.2.0]nonanyl, and the like.

In one embodiment, an alkenyl group is a univalent, straight or branched, substituted or unsubstituted, unsaturated hydrocarbon radical. In one embodiment, the alkenyl group has the general formula (notwithstanding optional substitution, higher degree of unsaturation, or the like) —CₙH₂ₙ₋₂. In one embodiment, n is 2-20 ((C₂-C₂₀) alkenyl), which may suitably include C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀ alkenyl groups. In one embodiment, the alkenyl group may be straight or branched, substituted or unsubstituted, have more than one degree of unsaturation, or any combination thereof. In one embodiment, one or more carbon atoms may be optionally and independently replaced with one or more heteroatoms such as O, S, N, B, or any combination thereof. In one embodiment, the alkenyl group is attached to the parent structure through one or more independent divalent intervening substituent groups. Some examples of alkenyl groups, which are not intended to be limiting, include ethenyl, 1-propenyl, 2-propenyl (allyl), iso-propenyl, 2-methyl-1-propenyl, 1-butenyl, 2-butenyl, alkadienes, alkatrienes, and the like.

In one embodiment, an alkynyl group is a univalent, straight or branched, substituted or unsubstituted, hydrocarbon radical that contains one or more carbon-carbon triple bond. In one embodiment, the alkenyl group has the general formula (notwithstanding optional substitution, higher degree of unsaturation, or the like)-CₙH₂ₙ₋₃. In one embodiment, n is 2-20 ((C₂-C₂₀) alkynyl), which may suitably include C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀ alkynyl groups. In one embodiment, the alkynyl group may be straight or branched, substituted or unsubstituted, have more than one degree of unsaturation, or any combination thereof. In one embodiment, one or more carbon atoms may be optionally and independently replaced with one or more heteroatoms such as O, S, N, B, or any combination thereof. In one embodiment, the alkynyl group is attached to the parent structure through one or more independent divalent intervening substituent groups. Some examples of alkynyl groups, which are not intended to be limiting, include alkadiynes, alkatriynes, ethynyl, propynyl, butynyl, and the like.

In one embodiment, an aryl group is a univalent, substituted or unsubstituted, monocyclic or polycyclic aromatic hydrocarbon radical. In one embodiment, an aryl group is a radical which, in accordance with Hackers theory, includes a cyclic, delocalized (4n+2) pi-electron system. In one embodiment the aryl group is a C₅-C₂₀ aryl group. The C₅-C₂₀ aryl group may suitably include C₅, C₆, C₇, C₈, C₉, C₁₀, C₁₁, C₁₂, C₁₃, C₁₄, C₁₅, C₁₆, C₁₇, C₁₈, C₁₉, and C₂₀ aryl groups. In one embodiment, the aryl group may be substituted or unsubstituted, be substituted with two or more groups that taken together form a cyclic group, or any combination thereof. In one embodiment, the aryl group is attached to the parent structure through one or more independent divalent intervening substituent groups. Some examples of aryl groups, which are not intended to be limiting, include phenyl, naphthyl, tetrahydronaphthyl, phenanthryl, pyrenyl, anthryl, indanyl, chrysyl, and the like.

In one embodiment, a heterocyclic group is a univalent, substituted or unsubstituted, saturated or unsaturated, mono- or polycyclic hydrocarbon radical that contains one or more heteroatoms in one or more of the rings. In one embodiment, the heterocyclic group is a C₃-C₂₀ cyclic group, in which one or more ring carbons is independently replaced with one or more heteroatoms. The C₃-C₂₀ heterocyclic group may suitably include C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀ cyclic groups in which one or more ring carbons is independently replaced with one or more heteroatoms. In one embodiment, the heteroatoms are selected from one or more of N, O, or S, or any combination thereof. In one embodiment, the N or S or both may be independently substituted with one or more substituents. In one embodiment, the heterocyclic group is substituted or unsubstituted, saturated or unsaturated, mono-, bi-, tri-, or poly-cyclic, or any combination thereof. In one embodiment, one or more hydrogens may be optionally and independently replaced by one or more substituent groups. In one embodiment, the heterocyclic group may include one or more carbon-carbon double bonds, carbon-carbon triple bonds, carbon-nitrogen double bonds, or any combination thereof. In one embodiment, the heterocyclic group is attached to the parent structure through one or more independent divalent intervening substituent groups. Some examples of heterocyclic groups, which are not intended to be limiting, include azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydrothiadiazinyl, morpholinyl, oxetanyl, tetrahydrodiazinyl, oxazinyl, oxathiazinyl, indolinyl, isoindolinyl, quinuclidinyl, chromanyl, isochromanyl, benzoxazinyl, tetrahydrofuran-2-yl, tetrahydrofuran-3-yl, imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperazin-1-yl, piperazin-2-yl, piperazin-3-yl, 1,3-oxazolidin-3-yl, isothiazolidine, 1,3-thiazolidin-3-yl, 1,2-pyrazolidin-2-yl, 1,3-pyrazolidin-1-yl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, 1,4-oxazin-2-yl, 1,2,5-oxathiazin-4-yl, and the like

In one embodiment, a heteroaryl group is univalent, substituted or unsubstituted, monocyclic or polycyclic aromatic hydrocarbon radical in which one or more ring carbons is independently replaced with one or more heteroatoms selected from O, S and N. In one embodiment, in addition to said heteroatom, the heteroaryl group may optionally have up to 1, 2, 3, or 4 N atoms in the ring. In one embodiment, the heteroaryl group is an aryl group in which one or more ring carbons are independently replaced with one or more heteroatoms. In one embodiment, a heteroaryl group is an aromatic radical, which contains one or more heteroatoms and which, in accordance with Hückel's theory, includes a cyclic, delocalized (4n+2) pi-electron system. In one embodiment, the heteroaryl group is a C₅-C₂₀ heteroaryl group. The C₅-C₂₀ heteroaryl group may suitably include C₅, C₆, C₇, C₈, C₉, C₁₀, C₁₁, C₁₂, C₁₃, C₁₄, C₁₅, C₁₆, C₁₇, C₁₈, C₁₉, and C₂₀ aryl groups in which one or more than one ring carbon is independently replaced with one or more heteroatoms. In one embodiment, the heteroaryl group may be substituted or unsubstituted, be substituted with two or more groups that taken together form a cyclic group, or any combination thereof. In one embodiment, the heteroaryl group is attached to the parent structure through one or more independent divalent intervening substituent groups. Some examples of heteroaryl groups, which are not intended to be limiting, include heteroaryl group includes pyridyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl, tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, indolyl, and the like.

In one embodiment, an aralkyl group is a univalent radical derived from one or more aryl groups attached to one or more of an alkylene group, cycloalkylene group, alkenylene group, alkynylene group, or combination thereof. The alkylene, cycloalkylene, alkenylene, and alkynylene groups are divalent radicals derived from the removal of hydrogen from the respective alkyl, cycloalkyl, alkenyl, or alkynyl groups. In this context, any combination of aryl group and alkyl, cycloalkyl, alkenyl, or alkynyl group is contemplated. In one embodiment, the aryl group is attached to the parent structure through one or more of the alkylene group, cycloalkylene group, alkenylene group, alkynylene group, or combination thereof as appropriate. In one embodiment, the aralkyl group may be attached to the parent structure through one or more independent divalent intervening substituent groups.

In one embodiment, a heteroaralkyl group is a univalent radical derived from one or more heteroaryl groups attached to one or more of an alkylene group, cycloalkylene group, alkenylene group, alkynylene group, or combination thereof. The alkylene, cycloalkylene, alkenylene, and alkynylene groups are divalent radicals derived from the removal of hydrogen from the respective alkyl, cycloalkyl, alkenyl, or alkynyl groups. In this context, any combination of heteroaryl group and alkyl, cycloalkyl, alkenyl, or alkynyl group is contemplated. In one embodiment, the heteroaryl group is attached to the parent structure through one or more of the alkylene group, cycloalkylene group, alkenylene group, alkynylene group, or combination thereof as appropriate. In one embodiment, the heteroaralkyl group may be attached to the parent structure through one or more independent divalent intervening substituent groups.

In one embodiment, a halo group is a univalent halogen radical or halogen-containing substituent group, e.g., one that is or contains one or more F, Br, Cl, I, or combination thereof. As used herein, the term “halogen” or “halo” includes fluoro, chloro, bromo, or iodo, or fluoride, chloride, bromide or iodide. In one embodiment, a halogen containing substituent group may suitably include a substituent group in which one or more hydrogen atoms are independently replaced with one or more halogens. In one embodiment, the halo group may be attached to the parent structure through one or more independent divalent intervening substituent groups.

In one embodiment, a hydroxy group is a univalent hydroxyl radical (—OH) or hydroxy-containing substituent group, e.g., one that is or contains one or more —OH. As used herein the term, “hydroxy” includes an —OH group. In one embodiment, a hydroxy-containing substituent group may suitably include a substituent group in which one or more hydrogen atoms are independently replaced with one or more —OH groups. In one embodiment, the hydroxyl group may be attached to the parent structure through one or more independent divalent intervening substituent groups.

In one embodiment, an oxo group is a univalent radical that contains an oxygen atom, ═O, doubly bonded to carbon or another element. In one embodiment, the oxo group suitably includes aldehydes, carboxylic acids, ketones, sulfonic acids, amides, esters, and combinations thereof. In one embodiment, the oxo group may be attached to the parent structure through one or more independent divalent intervening substituent groups.

In one embodiment, a mercapto or thiol group is a univalent —SR radical or an —SR-containing group. The R group is suitably chosen from any of the substituent groups. In one embodiment, the mercapto group may be attached to the parent structure through one or more independent divalent intervening substituent groups.

In one embodiment, an amino group is a univalent —NH₂ radical or an —NH₂— containing substituent group. In one embodiment, the amino group may be attached to the parent structure through one or more independent divalent intervening substituent groups.

In one embodiment, an alkylamino group is a univalent —NRH radical or an —NRH-containing substituent group. The R group is suitably chosen from any of the substituent groups. In one embodiment, the alkylamino group may be attached to the parent structure through one or more independent divalent intervening substituent groups.

In one embodiment, a dialkylamino group is a univalent —NRR radical or an —NRR-containing substituent group. The R groups may be the same or different and are suitably and independently chosen from any of the substituent groups. In one embodiment, the dialkylamino group may be attached to the parent structure through one or more independent divalent intervening substituent groups.

In one embodiment, a carbonyl group is a univalent radical that contains a —CR(═O) group. In one embodiment, the carbonyl group suitably includes aldehydes, ketones, and combinations thereof. The R group is suitably chosen from any of the substituent groups. In one embodiment, the carbonyl group may be attached to the parent structure through one or more independent divalent intervening substituent groups.

In one embodiment, a carboxylic acid group is a univalent —C(═O)OH radical or a —C(═O)OH-containing substituent group. In one embodiment, the carboxylic acid group may be attached to the parent structure through one or more independent divalent intervening substituent groups.

In one embodiment, a carboxylate group is a univalent —C(═O)O⁻ anion, —C(═O)OR, or —C(═O)OM, wherein M is a metal cation, or —C(═O)O⁻ anion, —C(═O)OR, or —C(═O)OM-containing substituent group. The R group is suitably chosen from any of the substituent groups. The metal cation is suitably chosen from Li, Na, K, and the like. In one embodiment, the carboxylate group may be attached to the parent structure through one or more independent divalent intervening substituent groups.

In one embodiment, an amidine group is a univalent —C(═NR)NRR radical or a —C(═NR)NRR-containing substituent group. The R groups may be the same or different and are suitably and independently chosen from any of the substituent groups. In one embodiment, the amidine group may be attached to the parent structure through one or more independent divalent intervening substituent groups.

In one embodiment, an amide group is a univalent -E(═O)NRR radical or a -E(═O)NRR-containing substituent group, in which E may be other than carbon, e.g., a chalcogen (e.g., S, Se, Te), or P. In one embodiment, the amide group suitably includes univalent lactams, peptides, phosphoramides, or sulfamides, —S(═O)₂NRR, —P(O)(OH)NRR, and the like. The R groups may be the same or different and are suitably and independently chosen from any of the substituent groups. In one embodiment, the amide group may be attached to the parent structure through one or more independent divalent intervening substituent groups.

In one embodiment, a carbamoyl group is a univalent —C(═O)NRR radical or a —C(═O)NRR-containing substituent group. The R groups may be the same or different and are suitably and independently chosen from any of the substituent groups. In one embodiment, the carbamoyl group may be attached to the parent structure through one or more independent divalent intervening substituent groups.

In one embodiment, a sulfonyl group is a univalent —S(═O)₂R radical or a —S(═O)₂R-containing substituent group. The R group is suitably chosen from any of the substituent groups. In one embodiment, the sulfonyl group may be attached to the parent structure through one or more independent divalent intervening substituent groups.

In one embodiment, an alkylthio or sulfide group is a univalent —SR radical an —SR-containing substituent group. The R group is suitably chosen from any of the substituent groups. In one embodiment, the alkylthio group may be attached to the parent structure through one or more independent divalent intervening substituent groups.

In one embodiment, an alkoxy group is a univalent radical derived from an —O-alkyl group. In one embodiment, the alkylthio group may be attached to the parent structure through one or more independent divalent intervening substituent groups.

In one embodiment, an aryloxy group is a univalent radical derived from an —O-aryl group. In one embodiment, the aryloxy group may be attached to the parent structure through one or more independent divalent intervening substituent groups.

In one embodiment, a heteroaryloxy group is a univalent radical derived from an —O-heteroaryl group. In one embodiment, the heteroaryloxy group may be attached to the parent structure through one or more independent divalent intervening substituent groups.

In one embodiment, an aralkoxy group is a univalent radical derived from an —O-aralkyl group. In one embodiment, the aralkoxy group may be attached to the parent structure through one or more independent divalent intervening substituent groups.

In one embodiment, a heteroaralkoxy group is a univalent radical derived from an —O-heteroaryl group. In one embodiment, the heteroaralkoxy group may be attached to the parent structure through one or more independent divalent intervening substituent groups.

In one embodiment, an alkylcarbonyl group is a univalent is radical derived from a -carbonyl-alkyl group. In one embodiment, the alkylcarbonyl group may be attached to the parent structure through one or more independent divalent intervening substituent groups.

In one embodiment, an alkoxycarbonyl group is a univalent radical derived from a -carbonyl-O-alkyl group. In one embodiment, the alkoxycarbonyl group may be attached to the parent structure through one or more independent divalent intervening substituent groups.

In one embodiment, an alkylaminocarbonyl group is a univalent radical derived from a -carbonyl-alkylamino group. In one embodiment, the heteroaralkoxy group may be attached to the parent structure through one or more independent divalent intervening substituent groups.

In one embodiment, a dialkylamino carbonyl group is a univalent radical derived from a -carbonyl-dialkylamino group. In one embodiment, the dialkylamino carbonyl group may be attached to the parent structure through one or more independent divalent intervening substituent groups.

In one embodiment, an arylcarbonyl group is a univalent radical derived from a -carbonyl-aryl group. In one embodiment, the arylcarbonyl group may be attached to the parent structure through one or more independent divalent intervening substituent groups.

In one embodiment, an aryloxycarbonyl group is a univalent radical derived from a -carbonyl-O-aryl group. In one embodiment, the aryloxycarbonyl group may be attached to the parent structure through one or more independent divalent intervening substituent groups.

In one embodiment, an alkylsulfonyl group is a univalent radical derived from a -sulfonyl-alkyl group. In one embodiment, the alkylsulfonyl group may be attached to the parent structure through one or more independent divalent intervening substituent groups.

In one embodiment, an arylsulfonyl group is a univalent radical derived from a -sulfonyl-aryl group. In one embodiment, the arylsulfonyl group may be attached to the parent structure through one or more independent divalent intervening substituent groups.

In one embodiment, a perhaloalkyl group is a univalent radical derived from a completely or substantially completely halogenated alkyl group. In one embodiment, the parhaloalkyl group may be attached to the parent structure through one or more independent divalent intervening substituent groups.

In one embodiment, a perhaloalkoxy group is a univalent radical derived from a completely or substantially completely halogenated alkoxy group. In one embodiment, the arylsulfonyl group may be attached to the parent structure through one or more independent divalent intervening substituent groups.

In one embodiment, a perhalocycloalkyl group is a univalent radical derived from a completely or substantially completely halogenated cycloalkyl group. In one embodiment, the perhalocycloalkyl group may be attached to the parent structure through one or more independent divalent intervening substituent groups.

In one embodiment, a perhaloalkenyl group is a univalent radical derived from a completely or substantially completely halogenated alkenyl group. In one embodiment, the perhaloalkenyl group may be attached to the parent structure through one or more independent divalent intervening substituent groups.

In one embodiment, a perhaloalkynyl group is a univalent radical derived from a completely or substantially completely halogenated alkynyl group. In one embodiment, the perhaloalkynyl group may be attached to the parent structure through one or more independent divalent intervening substituent groups.

In one embodiment, a perhaloaryl group is a univalent radical derived from a completely or substantially completely halogenated aryl group. In one embodiment, the perhaloaryl group may be attached to the parent structure through one or more independent divalent intervening substituent groups.

In one embodiment, a perhaloaralkyl group is a univalent radical derived from a completely or substantially completely halogenated aralkyl group. In one embodiment, the perhaloaralkyl group may be attached to the parent structure through one or more independent divalent intervening substituent groups.

In one embodiment, an alkylcarbonyloxy group is a univalent radical derived from an —O-carbonyl-alkyl group. In one embodiment, the alkylcarbonyloxy group may be attached to the parent structure through one or more independent divalent intervening substituent groups.

In one embodiment, an alkoxycarbonyloxy group is a univalent radical derived from an —O-carbonyl-O-alkyl group. In one embodiment, the alkoxycarbonyloxy group may be attached to the parent structure through one or more independent divalent intervening substituent groups.

In one embodiment, an alkylsulfonyloxy group is a univalent radical derived from an —O-sulfonyl-alkyl group. In one embodiment, the alkylsulfonyloxy group may be attached to the parent structure through one or more independent divalent intervening substituent groups.

In one embodiment, an alkoxysulfonyloxy group is a univalent radical derived from an —O-sulfonyl-O-alkyl group. In one embodiment, the alkoxysulfonyloxy group may be attached to the parent structure through one or more independent divalent intervening substituent groups.

In one embodiment, an arylcarbonyloxy group is a univalent radical derived from an —O-carbonyl-aryl group. In one embodiment, the arylcarbonyloxy group may be attached to the parent structure through one or more independent divalent intervening substituent groups.

In one embodiment, an aryloxycarbonyloxy group is a univalent radical derived from an —O-carbonyl-O-aryl group group. In one embodiment, the aryloxycarbonyloxy group may be attached to the parent structure through one or more independent divalent intervening substituent groups.

In one embodiment, an arylsulfonyloxy group is a univalent radical derived from an —O-sulfonyl-aryl group. In one embodiment, the arylsulfonyloxy group may be attached to the parent structure through one or more independent divalent intervening substituent groups.

In one embodiment, an aryloxysulfonyloxy group is a univalent radical derived from an —O-sulfonyl-O-aryl group. In one embodiment, the aryloxysulfonyloxy group may be attached to the parent structure through one or more independent divalent intervening substituent groups.

In one embodiment, referring to two groups taken together to form a cyclic group, the cyclic group may be suitably derived from a divalent cycloalkylene group or divalent heterocyclic group. The divalent cycloalkylene and heterocyclic groups may be suitably derived from the respective cycloalkyl or heterocyclic groups.

In one embodiment, referring to two groups taken together to form an aryl group, the aryl group may be suitably derived from a divalent arylene group or divalent heteroarlyene group. The divalent arylene and heteroarylene groups may be suitably derived from the respective aryl or heteroaryl groups.

In one embodiment, referring to the replacement of one or more than one atom in each group with one or more heteroatoms, the heteroatoms may be suitably chosen from N, O, P, S, B, or any combination thereof as appropriate.

In one embodiment, the structure

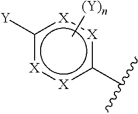

may have one of the following three structures:

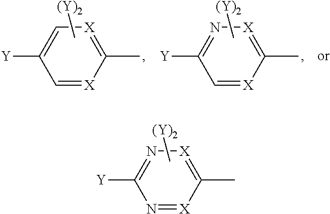

wherein each X is independently carbon or nitrogen, and wherein X is carbon, it independently comprises a Y substituent. In the three structures shown above, in one embodiment, the X's may be carbon, each carbon independently comprising a Y substituent.

In one embodiment, the structure

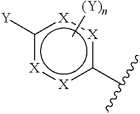

may have one of the following structures:

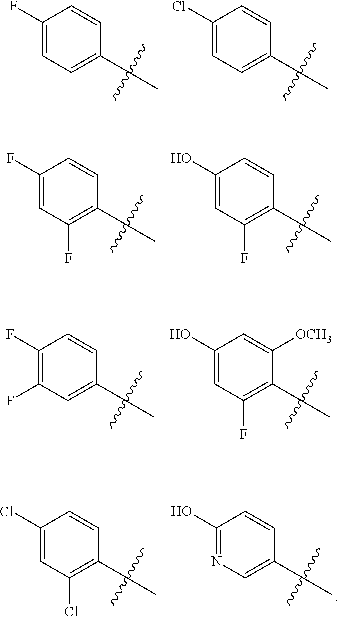

In one embodiment, Y may be an alkyl group, a cycloalkyl group, a halo group, a perfluoroalkyl group, a perfluoroalkoxy group, an alkenyl group, an alkynyl group, a hydroxy group, an oxo group, a mercapto group, an alkylthio group, an alkoxy group, an aryl group, a heteraryl group, an aryloxy group, a heteroaryloxy group, an aralkyl group, a heteroaralkyl group, an aralkoxy group, a heteroaralkoxy group, an HO—(C═O)— group, an amino group, an alkylamino group, a dialkylamino group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, or an arylsulfonyl group, or have the following structure:

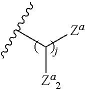

in which each Z^{a} is independently either hydrogen, hydroxyl, halogen, or a substituent group; and

“j” is independently either zero or an integer from one to four.

In one embodiment, the structure:

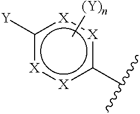

has the following structure:

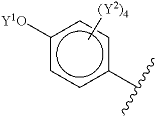

in which each Y¹ is independently a hydrogen or (C₁-C₆)alkyl; and

each Y² is independently a Y¹, hydroxyl group, halo group, —N₃, —CN, —SH, or —N(Y¹)₂.

In one embodiment, the compound having formula (V) has the following structure:

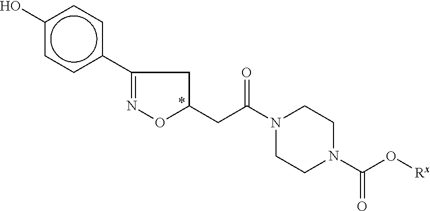

wherein R^{X} is a (C₁-C₆)alkyl, (C₂-C₆)alkenyl, (C₂-C₆)alkynyl, phenyl, (C₅-C₁₄)aryl, (C₄-C₁₄)heteroaryl, (C₂-C₁₄)heterocyclic or (C₃-C₁₀)cycloalkyl group.

In one embodiment, the compound having formula (V) has the following structure:

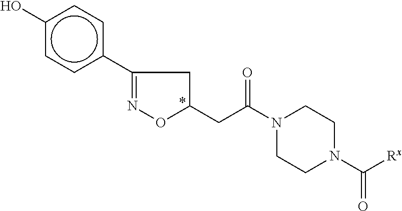

wherein R^{X} is a (C₁-C₆)alkyl, (C₂-C₆)alkenyl, (C₂-C₆)alkynyl, phenyl, (C₅-C₁₄)aryl, (C₄-C₁₄)heteroaryl, (C₂-C₁₄)heterocyclic or (C₃-C₁₀)cycloalkyl group.

In one embodiment, the compound having formula (V) has the following structure:

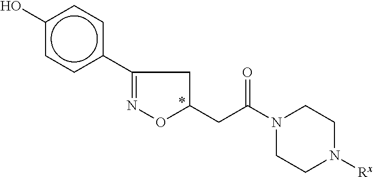

wherein R^{X} is a sulfonyl, carbonyl, (C₁-C₆)alkylsulfonyl, (C₁-C₆)alkylcarbonyl, (C₅-C₁₄)arylsulfonyl, (C₅-C₁₄)arylcarbonyl, (C₁-C₆)alkyl, (C₂-C₆)alkenyl, (C₂-C₆)alkynyl, phenyl, (C₅-C₁₄)aryl, (C₄-C₁₄)heteroaryl, (C₂-C₁₄)heterocyclic or (C₃-C₁₀)cycloalkyl group.

In one embodiment, the compound having formula (V) has the following structure:

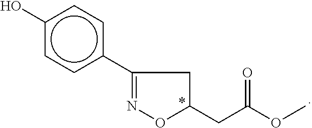

In one embodiment, the substituent groups described herein may be suitably and independently chosen from one or more of a hydrogen, an azido group, a carbamido group, a carbazoyl group, a cyanato group, a cyano group, an isocyanato group, an isocyano group, a hydroxamino group, a guanidino group, a guanyl group, an imino group, a nitro group, a phospho group, a phosphate group, a phosphine group, a sulfo group, a sulfate group, a sulfonyl group, a carbonyl group, a carboxylic acid group, a carboxylate group, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, an alkylcarbonyloxy group, an alkoxycarbonyloxy group, an alkylsulfonyloxy group, an alkoxysulfonyloxy group, an arylcarbonyloxy group, an aryloxycarbonyloxy group, an arylsulfonyloxy group, an aryloxysulfonyloxy group, an a perhaloalkyl group, a perhaloalkoxy group, a perhalocycloalkyl group, a perhaloalkenyl group, a perhaloalkynyl group, a perhaloaryl group, a perhaloaralkyl group, or combination thereof. Univalent residues or divalent intervening residues of any substituent group or combination thereof may be suitably used as appropriate.

In one embodiment, the divalant intervening substituent groups may be suitably and independently chosen from one or more of an azo group, an azino group, an azoxy group, a carbonyl group, a dioyl group, a diazoamino group, a disulfinyl group, a dithio group, an oxy group, a hydrazo group, an oxalyl group, a sulfonyl group, a a thiocarbonyl group, a thionyl group, a phosphono ester group, a carboxylate group, a thio group; divalent residues of one or more of the following groups: an alkyl group, a cycloalkyl group, an alkenyl group, an alkynyl group, an alkylthio group, an alkyloxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, an alkylcarbonyloxy group, an alkoxycarbonyloxy group, an alkylsulfonyloxy group, an alkoxysulfonyloxy group, an arylcarbonyloxy group, an aryloxycarbonyloxy group, an arylsulfonyloxy group, an aryloxysulfonyloxy group, an a perhaloalkyl group, a perhaloalkoxy group, a perhalocycloalkyl group, a perhaloalkenyl group, a perhaloalkynyl group, a perhaloaryl group, a perhaloaralkyl group, combination thereof; or combination thereof.

In one embodiment, referring to a base, it may be a compound with a lone pair capable of bonding to a hydrogen ion. In one embodiment, the base may be one or more of an inorganic hydroxide salt, inorganic oxide salt, amine, amine salt, conjugate base of an acid, or combination thereof. Some examples of a base, which are not intended to be limiting, include KOH, Ba(OH)₂, CsOH, NaOH, Ca(OH)₂, LiOH, Mg(OH)₂, n-BuLi, LiN(iPr)₂, LiNEt₂, NaNH₂, NaH, LiN(Si(CH₃)₃)₂, NaHCO₃, Na₂CO₃, NH₃, monoalkylamine, dialkylamine, trialkylamine, pyridine, imidazole, benzimidazole, histidine, hexachlorophosphazene, bis(triphenylphosphine)iminium chloride, and combinations thereof.

In one embodiment, referring to an aqueous bleach, it may be an aqueous solution of a compound capable of replacing a C—H bond with a C-halogen bond. In one embodiment, the aqueous bleach may be an aqueous solution of one or more of sodium hypochlorite, calcium hypochlorite, or a combination thereof.

In one embodiment, referring to an electrophile, it may be a reagent attracted to electrons that participates in a chemical reaction by accepting an electron pair in order to bond to a nucleophile.

In one embodiment, referring to an acid, it may be a compound that donates a hydrogen ion to another compound. In one embodiment, an acid may be one or more of a hydrogen halide, halogen oxyacid, inorganic compound that donate a hydrogen ion, sulfonic acid, carboxylic acid, or combination thereof. Some examples of an acid, which are not intended to be limiting, include HF, HCl, HBr, HI, HClO, HClO₃, HClO₄, HBrO, HBrO₃, HBrO₄, H₂SO₄, HFSO₃, HNO₃, H₃PO₄, H₃BO₃, HBF₄, HPF₆, MeSO₃H, EtSO₃H, PhSO₃H, p-CH₃C₆H₄SO₃H, CF₃SO₃H, HCO₂H, CH₃CO₂H, CH₂ClCO₂H, CHCl₂CO₂H, CCl₃CO₂H, CF₃CO₂H, ascorbic acid, benzoic acid, citric acid, gluconic acid, lactic acid, malic acid, mandelic acid, oxalic acid, pivalic acid, tartaric acid, or combination thereof.

In one embodiment, referring to a trialkylamine, it may be an amine molecule with a combination of three substituents selected from either alkyl or aryl groups. In one embodiment, a trialkyl amine may be a trisubstituted amine which contains a free lone pair of electrons and is substituted with a combination of three alkyl and aryl groups. One example of a trialkylamine, which is not intended to be limiting, includes a compound of formula RR′R″N wherein R, R′, and R″ are independently selected from alkyl, aryl, heteroaryl, heteroalkyl.

In one embodiment, referring to an aromatic N-containing heterocycle, it may be a cyclic group containing more than one kind of atom in addition to an N and which is aromatic. In one embodiment, an aromatic N-containing heterocycle may be a cyclic group containing a combination of carbon, nitrogen, oxygen an sulfur in an aromatic ring. In one embodiment, an aromatic N-containing heterocycle may be a cyclic group of 5-9 atoms in an aromatic ring with at least one nitrogen.

In one embodiment, referring to an organometallic compound, it may be a compound containing and alkyl, heteroalkyl, heteroaryl, or aryl radical bonded to a metal. In one embodiment, the organometallic compound may be a compound containing an alkyl heteroalkyl, heteroaryl or aryl radical bonded to Li, Na, K, Rb, Cs, B, Al, Fe, Ru, Rh, Sn, Cr, or combination thereof. In one embodiment, the organometallic compound may be a compound containing an alkyl, heteroalkyl, heteroaryl or aryl radical or combination thereof bonded to Li, Na, K, Rb, Cs, B, Al, Sn, or combination thereof.

In one embodiment, referring to a nucleophilic O, N, S, C, B which forms a new bond with electrophile, it may be a reagent containing an O, N, S, C, or B with an available lone pair that participates in a chemical reaction by donating an electron pair in order to bond to a electrophile.

In one embodiment, referring to a new bond, it may be a chemical bond formed by the sharing of one or more electrons, especially pairs of electrons, between atoms. In one embodiment, the new bond may be a covalent bond.

In one embodiment, referring to an acid halide, it may be an organic compound of formula RCOX wherein X is a halogen and R is an alkyl, heteroalkyl, heteroaryl or aryl group. In one embodiment, the acid halide may be an organic compound of formula RCOX wherein X is Cl, Br, or I and R is an alkyl heteroalkyl, heteroaryl or aryl group. In one embodiment, the acid halide may be an organic compound of formula RCOX wherein R is an alkyl, heteroalkyl, aryl, or heteroaryl group and X is Cl.

In one embodiment, referring to a hydroxylamine salt, it may be a compound of formula (NH₃⁺OH)X⁻ used as a nucleophile wherein X is a halogen. In one embodiment, the hydroxylamine salt may be a compound of formula (NH₃⁺OH)Cl⁻, (NH₃⁺OH)Br⁻, or combination thereof.

In one embodiment, referring to a halogenating agent, it may be a compound which introduces a chlorine, bromine or iodine into the target molecule. In one embodiment, the halogenating agent is a compound which replaces a carbon-hydrogen bond with a carbon-X bond wherein X is chlorine, bromine, or iodine. Some examples of a halogenating agent, which are not intended to be limiting, include oxalyl chloride, oxalyl bromide, N-bromosuccinimide, N-chlorosuccinimide, N-iodosuccinimide, thionyl chloride, Cl₂, Br₂, I₂, or combination thereof.

In one embodiment, referring to a carboxylic acid, it may be a compound of the formula RCO₂H wherein R is an alkyl, heteroalkyl, heteroaryl, or aryl group. Some examples of a carboxylic acid, which are not intended to be limiting, include HCO₂H, CH₃CO₂H, CH₂ClCO₂H, CHCl₂CO₂H, CCl₃CO₂H, CF₃CO₂H, ascorbic acid, benzoic acid, citric acid, gluconic acid, lactic acid, malic acid, mandelic acid, oxalic acid, pivalic acid, tartaric acid, or combination thereof.

In one embodiment, referring to a sulfonic acid, it may be a compound of the formula RSO₃H wherein R is an alkyl, heteroalkyl, heteroaryl, or aryl group. Some examples of a sulfonic acid, which are not intended to be limiting, include MeSO₃H, EtSO₃H, PhSO₃H, p-CH₃C₆H₄SO₃H, CF₃SO₃H, or combination thereof.

In one embodiment, the compounds have utility in pharmacological compositions for the treatment and/or prevention of many diseases and disorders characterized by a MIF response, whereby MIF is released from cellular sources and MIF production is enhanced. If desired, the compound can be administered to a human patient by itself or in pharmaceutical compositions where it may be mixed with suitable carriers or excipients at doses to treat or ameliorate various conditions characterized by MIF release. A therapeutically effective dose may refer to that amount of the compound sufficient to inhibit MIF tautomerase activity and MIF bioactivity, it being understood that such inhibition may occur at different concentrations such that a person skilled in the art could determine the required dosage of compound to inhibit the target MIF activity. Therapeutically effective doses may be administered alone or as adjunctive therapy in combination with other treatments, such as steroidal or non-steroidal anti-inflammatory agents, or anti-tumor agents. Techniques for the formulation and administration of the compounds of the instant application may be found in Remington's Pharmaceutical Sciences, Mack Publishing Co., Easton, Pa., latest addition.

Suitable routes of administration may, for example, include oral, rectal, transmucosal, buccal, intravaginal, or intestinal administration; parenteral delivery, including intramuscular, subcutaneous, intramedullary injections, as well as intrathecal, direct intraventricular, intravenous, intraperitoneal, intranasal, or intraocular injections, and optionally in a depot or sustained release formulation. Furthermore, one may administer the compound in a targeted drug delivery system, for example in a liposome.

The pharmaceutical compositions and compounds may be manufactured in a manner that is itself known, e.g., by means of conventional mixing, dissolving, dragee-making, levitating, emulsifying, encapsulating, entrapping, or lyophilizing processes. The pharmaceutical compositions thus may be formulated in conventional manner using one or more physiologically acceptable carriers comprising excipients and auxiliaries that facilitate processing of the active compounds into preparations, which can be used pharmaceutically. Proper formulation may be dependent upon the route of administration chosen.

Any combination of one or more compounds, salts, prodrugs, metabolites, isotopically-labeled compounds, tautomers, isomers, and/or atropisomers is possible in the pharmaceutical composition.

For injection, the compounds may be formulated in aqueous solutions, preferably in physiologically compatible buffers, such as Hank's solution, Ringer's solution, or physiological saline buffer. For transmucosal administration, penetrants appropriate to the barrier to be permeated are used in the formulation. Such penetrants are known in the art.

For oral administration, the compounds can be formulated readily by combining the active compounds with pharmaceutically acceptable carriers well known to those in the art. Such carriers enable the compounds to be formulated as tablets, pills, dragees, capsules, liquids, gels, syrups, slurries, suspensions and the like, for oral ingestion by a patient to be treated. Pharmaceutical preparations for oral use can be obtained by combining the compound with a solid excipient, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as polyionic block (co)polymer, sodium carboxymethyl cellulose, sorbitol, or dextran. Optionally, the suspension may also contain suitable stabilizers or agents which increase the solubility of the compounds to allow for the preparation of highly concentrated solutions, e.g., polyionic block (co)polymers.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

The compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various forms of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds may be provided as salts with pharmaceutically compatible counterions. Pharmaceutically compatible salts may be formed with many acids, including but not limited to hydrochloric, sulfuric, acetic, lactic, tartaric, malic, succinic, etc.; or bases. Salts tend to be more soluble in aqueous or other protonic solvents than are the corresponding free base forms. Examples of pharmaceutically acceptable salts, carriers or excipients are well known to those skilled in the art and can be found, for example, in Remington's Pharmaceutical Sciences, 18th Edition, A. R. Gennaro, Ed., Mack Publishing Co., Easton, Pa. (1990). Such salts include, but are not limited to, sodium, potassium, lithium, calcium, magnesium, iron, zinc, hydrochloride, hydrobromide, hydroiodide, acetate, citrate, tartrate and maleate salts, and the like.

Generally, pharmaceutical compositions contain the active compound in an effective amount to achieve their intended purpose. In one embodiment, a therapeutically effective amount means an amount effective to prevent or inhibit development or progression of a disease characterized by MIF release and production in the subject being treated. Determination of the effective amounts is well within the capability of those skilled in the art, especially in light of the detailed disclosure provided herein.

In one embodiment, a method is provided, which comprises inhibiting the production of at least one cytokine selected from the group consisting of MIF, IL-1, IL-2, IL-6, IL-8, IFN-γ, TNF, and a combination thereof in a mammalian subject in need thereof by administering an inhibiting-effective amount of the compound to the subject.

In one embodiment, the mammalian subject is a human, an animal, a domesticated animal, a livestock, a cow, a pig, a horse, and the like.

In one embodiment, a method is provided, which comprises inhibiting an ERK/MAP pathway in a mammalian subject in need thereof by administering an inhibiting-effective amount of the compound to the subject.

In one embodiment, a method is provided, which comprises treating or preventing at least one disease selected from the group consisting of psoriatic arthritis, Reiter's syndrome, rheumatoid arthritis, gout, traumatic arthritis, rubella arthritis, acute synovitis, rheumatoid spondylitis, osteoarthritis, gouty arthritis and other arthritic conditions, sepsis, septic shock, endotoxic shock, gram negative sepsis, toxic shock syndrome, Alzheimer's disease, stroke, ischemic stroke, hemorrhagic stroke, neurotrauma/closed head injury, asthma, adult respiratory distress syndrome, chronic obstructive pulmonary disease, cerebral malaria, malaria, leishmania, meningitis, chronic pulmonary inflammatory disease, silicosis, pulmonary sarcostosis, bone resorption disease, osteoporosis, restenosis, cardiac reperfusion injury, brain reperfusion injury, renal reperfusion injury, chronic renal failure, thrombosis, glomerularonephritis, nephritis, diabetes, diabetic retinopathy, macular degeneration, graft vs. host reaction, allograft rejection, inflammatory bowel disease, Crohn's disease, ulcerative colitis, neurodegenerative disease, multiple sclerosis, muscle degeneration, diabetic retinopathy, macular degeneration, tumor growth and metastasis, angiogenic disease, rhinovirus infection, peroral disease, gingivitis, periodontitis, eczema, contact dermatitis, psoriasis, sunburn, conjunctivitis, post-operative ileus, bladder cancer, prostate cancer, ovarian cancer, breast cancer, skin cancer, cardiovascular disease, solid tumor, teratoma, Type 1 diabetes, Type 2 diabetes, atherosclerosis, multiple sclerosis, myocardial infarction, arteriosclerosis, and a combination thereof, in a mammal by administering one or compounds or compositions including such compounds to the mammal.

In one embodiment, a method is provided, which comprises inhibiting the production of at least one cytokine selected from the group consisting of MIF, IL-1, IL-2, IL-6, IL-8, IFN-γ, TNF, and a combination thereof in a cell culture by contacting an inhibiting-effective amount of the compound with at least one cell in the cell culture.

In one embodiment, the cell is a human cell.

Several examples of compounds which may be prepared in accordance with the processes described herein may be found in the table below, which is not intended to be limiting:

| CPSI Code | Structure | MF | MW |
|---|---|---|---|
| 00001-1 | 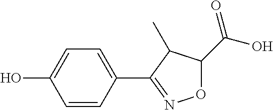 [SMILES: CC1C(c2ccc(O)cc2)=NOC1C(=O)O] | C11H11NO4 | 221.21 |
| 00002-1 | 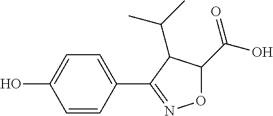 [SMILES: CC(C)C1C(c2ccc(O)cc2)=NOC1C(=O)O] | C13H15NO4 | 249.29 |
| 00003-1 | 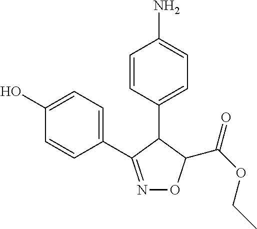 [SMILES: CCOC(=O)C1ON=C(c2ccc(O)cc2)C1c1ccc(N)cc1] | C18H18N2O4 | 326.36 |
| 00004-1 | 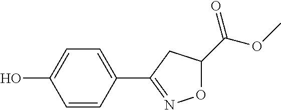 [SMILES: COC(=O)C1CC(c2ccc(O)cc2)=NO1] | C11H11NO4 | 221.21 |
| 00005-1 | 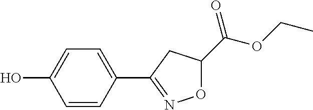 [SMILES: CCOC(=O)C1CC(c2ccc(O)cc2)=NO1] | C12H13NO4 | 235.24 |
| 00006-1 | 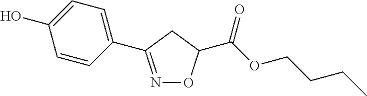 [SMILES: CCCCOC(=O)C1CC(c2ccc(O)cc2)=NO1] | C14H17NO4 | 263.3 |
| 00007-1 | 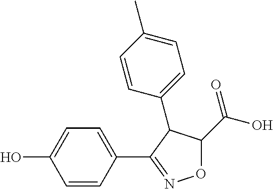 [SMILES: Cc1ccc(C2C(c3ccc(O)cc3)=NOC2C(=O)O)cc1] | C17H15NO4 | 297.31 |
| 00008-1 | 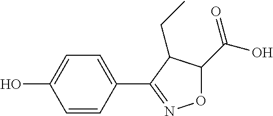 [SMILES: CCC1C(c2ccc(O)cc2)=NOC1C(=O)O] | C12H13NO4 | 235.24 |
| 00009-1 | 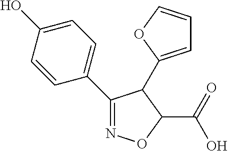 [SMILES: O=C(O)C1ON=C(c2ccc(O)cc2)C1c1ccco1] | C14H11NO5 | 273.24 |
| 00010-1 | 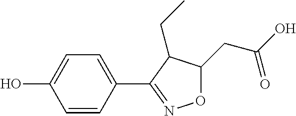 [SMILES: CCC1C(c2ccc(O)cc2)=NOC1CC(=O)O] | C13H15NO4 | 249.27 |
| 00011-1 | 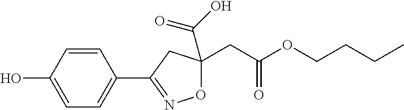 [SMILES: CCCCOC(=O)CC1(C(=O)O)CC(c2ccc(O)cc2)=NO1] | C16H19NO6 | 321.33 |
| 00012-1 | 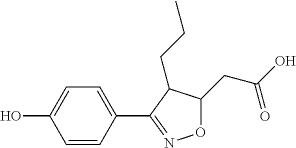 [SMILES: CCCC1C(c2ccc(O)cc2)=NOC1CC(=O)O] | C14H17NO4 | 263.3 |
| 00013-1 | 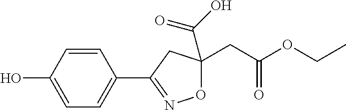 [SMILES: CCOC(=O)CC1(C(=O)O)CC(c2ccc(O)cc2)=NO1] | C14H15NO6 | 293.27 |
| 00014-1 | 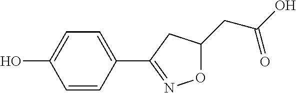 [SMILES: O=C(O)CC1CC(c2ccc(O)cc2)=NO1] | C11H11NO4 | 221.21 |
| 00015-1 | 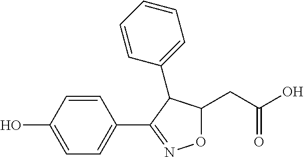 [SMILES: O=C(O)CC1ON=C(c2ccc(O)cc2)C1c1ccccc1] | C17H15NO4 | 297.31 |
| 00016-1 | 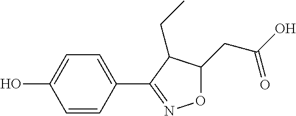 [SMILES: CCC1C(c2ccc(O)cc2)=NOC1CC(=O)O] | C13H15NO4 | 249.29 |
| 00017-1 | 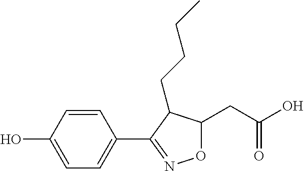 [SMILES: CCCCC1C(c2ccc(O)cc2)=NOC1CC(=O)O] | C15H19NO4 | 277.32 |
| 00018-1 | 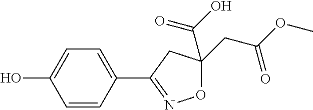 [SMILES: COC(=O)CC1(C(=O)O)CC(c2ccc(O)cc2)=NO1] | C13H13NO6 | 279.25 |
| 00019-1 | 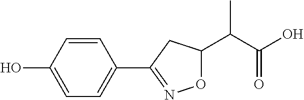 [SMILES: CC(C(=O)O)C1CC(c2ccc(O)cc2)=NO1] | C12H13NO4 | 235.24 |
| 00020-1 | 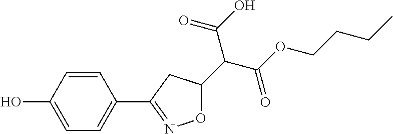 [SMILES: CCCCOC(=O)C(C(=O)O)C1CC(c2ccc(O)cc2)=NO1] | C16H19NO6 | 321.33 |
| 00021-1 | 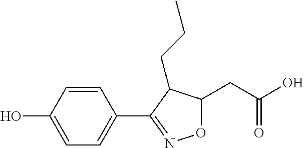 [SMILES: CCCC1C(c2ccc(O)cc2)=NOC1CC(=O)O] | C14H17NO4 | 263.29 |
| 00022-1 | 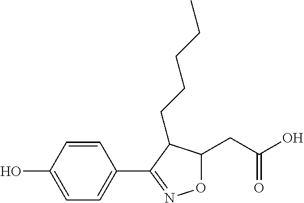 [SMILES: CCCCCC1C(c2ccc(O)cc2)=NOC1CC(=O)O] | C16H21NO4 | 291.34 |
| 00023-1 | 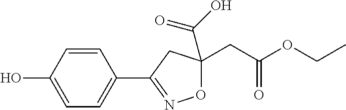 [SMILES: CCOC(=O)CC1(C(=O)O)CC(c2ccc(O)cc2)=NO1] | C14H15NO6 | 293.27 |
| 00024-1 | 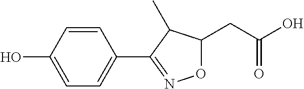 [SMILES: CC1C(c2ccc(O)cc2)=NOC1CC(=O)O] | C12H13NO4 | 235.24 |
| 00025-1 | 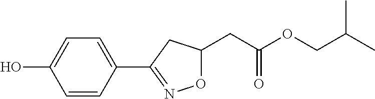 [SMILES: CC(C)COC(=O)CC1CC(c2ccc(O)cc2)=NO1] | C15H19NO4 | 277.32 |
| 00026-1 | 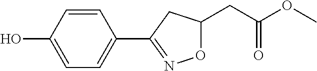 [SMILES: COC(=O)CC1CC(c2ccc(O)cc2)=NO1] | C12H13NO4 | 235.24 |
| 00027-1 | 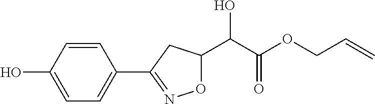 [SMILES: C=CCOC(=O)C(O)C1CC(c2ccc(O)cc2)=NO1] | C14H15NO5 | 277.27 |
| 00028-1 | 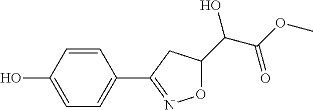 [SMILES: COC(=O)C(O)C1CC(c2ccc(O)cc2)=NO1] | C12H13NO5 | 251.24 |
| 00029-1 | 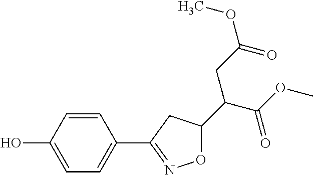 [SMILES: COC(=O)CC(C(=O)OC)C1CC(c2ccc(O)cc2)=NO1] | C15H17NO6 | 307.31 |
| 00030-1 | 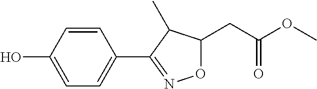 [SMILES: COC(=O)CC1ON=C(c2ccc(O)cc2)C1C] | C13H15NO4 | 249.26 |
| 00031-1 | 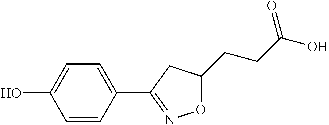 [SMILES: O=C(O)CCC1CC(c2ccc(O)cc2)=NO1] | C12H13NO4 | 235.24 |
| 00032-1 | 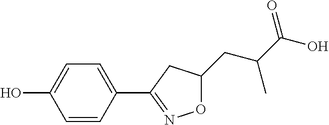 [SMILES: CC(CC1CC(c2ccc(O)cc2)=NO1)C(=O)O] | C13H15NO4 | 249.27 |
| 00033-1 | 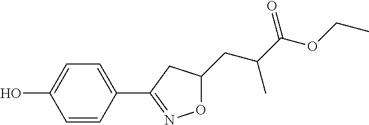 [SMILES: CCOC(=O)C(C)CC1CC(c2ccc(O)cc2)=NO1] | C15H19NO4 | 277.32 |
| 00034-1 | 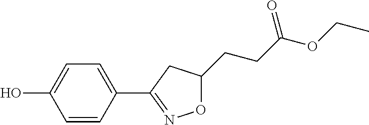 [SMILES: CCOC(=O)CCC1CC(c2ccc(O)cc2)=NO1] | C14H17NO4 | 263.3 |
| 00044-1 | 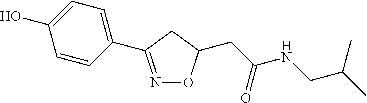 [SMILES: CC(C)CNC(=O)CC1CC(c2ccc(O)cc2)=NO1] | C15H20N2O3 | 276.33 |
| 00045-1 | 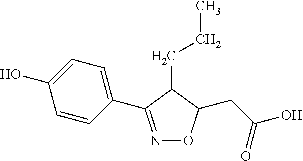 [SMILES: CCCC1C(c2ccc(O)cc2)=NOC1CC(=O)O] | C14H17NO4 | 263.29 |
| 00046-1 | 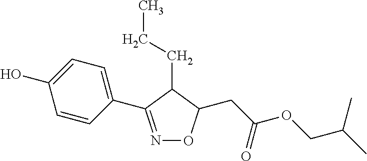 [SMILES: CCCC1C(c2ccc(O)cc2)=NOC1CC(=O)OCC(C)C] | C19H25NO4 | 319.4 |
| 00047-1 | 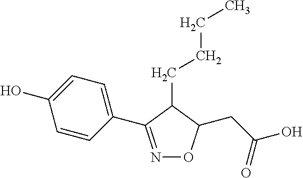 [SMILES: CCCCC1C(c2ccc(O)cc2)=NOC1CC(=O)O] | C15H19NO4 | 277.32 |
| 00048-1 | 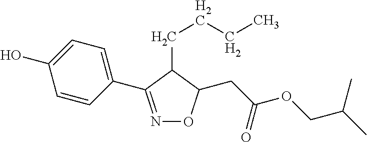 [SMILES: CCCCC1C(c2ccc(O)cc2)=NOC1CC(=O)OCC(C)C] | C19H27NO4 | 333.42 |
| 00057-1 | 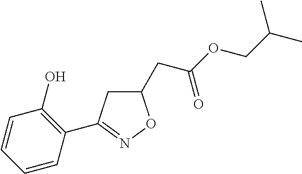 [SMILES: CC(C)COC(=O)CC1CC(c2ccccc2O)=NO1] | C15H19NO4 | 277.32 |
| 00058-1 | 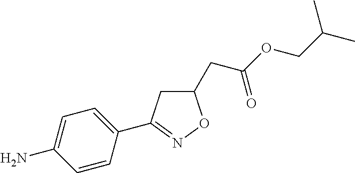 [SMILES: CC(C)COC(=O)CC1CC(c2ccc(N)cc2)=NO1] | C15H20N2O3 | 276.33 |
| 00059-1 | 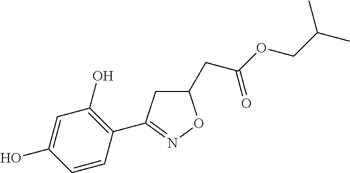 [SMILES: CC(C)COC(=O)CC1CC(c2ccc(O)cc2O)=NO1] | C15H19NO5 | 293.32 |
| 00060-1 | 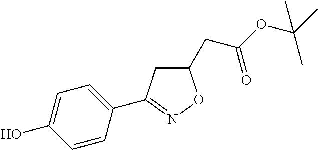 [SMILES: CC(C)(C)OC(=O)CC1CC(c2ccc(O)cc2)=NO1] | C15H19NO4 | 277.32 |
| 00061-1 | 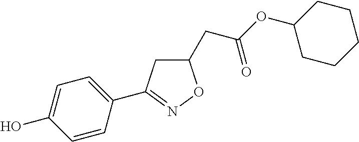 [SMILES: O=C(CC1CC(c2ccc(O)cc2)=NO1)OC1CCCCC1] | C17H21NO4 | 303.35 |
| 00062-1 | 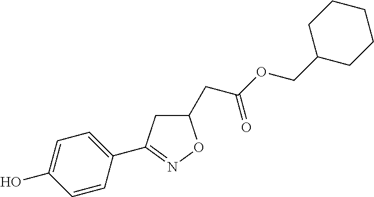 [SMILES: O=C(CC1CC(c2ccc(O)cc2)=NO1)OCC1CCCCC1] | C18H23NO4 | 317.38 |
| 00063-1 | 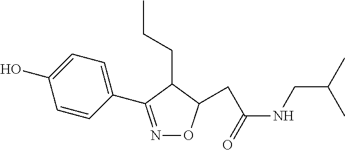 [SMILES: CCCC1C(c2ccc(O)cc2)=NOC1CC(=O)NCC(C)C] | C18H26N2O3 | 318.41 |
| 00064-1 | 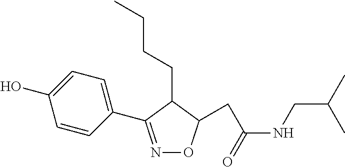 [SMILES: CCCCC1C(c2ccc(O)cc2)=NOC1CC(=O)NCC(C)C] | C19H28N2O3 | 332.44 |
| 00065-1 | 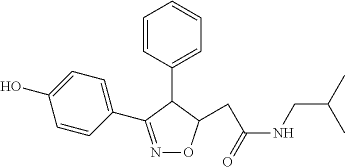 [SMILES: CC(C)CNC(=O)CC1ON=C(c2ccc(O)cc2)C1c1ccccc1] | C21H24N2O3 | 352.43 |
| 00066-1 | 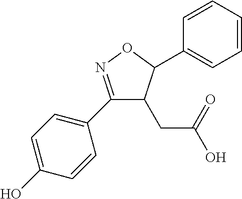 [SMILES: O=C(O)CC1C(c2ccc(O)cc2)=NOC1c1ccccc1] | C17H15NO4 | 297.31 |
| 00067-1 | 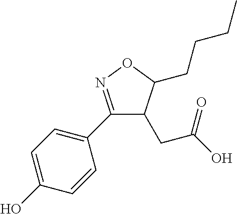 [SMILES: CCCCC1ON=C(c2ccc(O)cc2)C1CC(=O)O] | C15H19NO4 | 277.32 |
| 00068-1 | 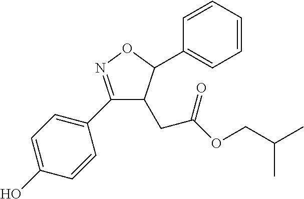 [SMILES: CC(C)COC(=O)CC1C(c2ccc(O)cc2)=NOC1c1ccccc1] | C21H24N2O3 | 353.41 |
| 00069-1 | 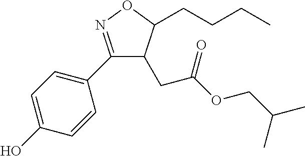 [SMILES: CCCCC1ON=C(c2ccc(O)cc2)C1CC(=O)OCC(C)C] | C19H28N2O3 | 333.42 |
| 00070-1 | 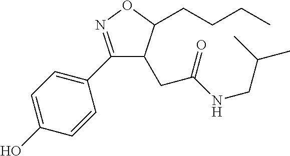 [SMILES: CCCCC1ON=C(c2ccc(O)cc2)C1CC(=O)NCC(C)C] | C19H28N2O3 | 332.44 |
| 00071-1 | 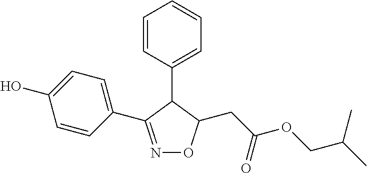 [SMILES: CC(C)COC(=O)CC1ON=C(c2ccc(O)cc2)C1c1ccccc1] | C21H23NO4 | 353.41 |
| 00072-1 | 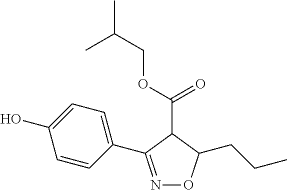 [SMILES: CCCC1ON=C(c2ccc(O)cc2)C1C(=O)OCC(C)C] | C17H23NO4 | 305.37 |
| 00073-1 | 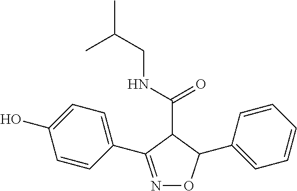 [SMILES: CC(C)CNC(=O)C1C(c2ccc(O)cc2)=NOC1c1ccccc1] | C20H22N2O3 | 338.40 |
| 00074-1 | 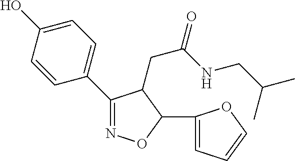 [SMILES: CC(C)CNC(=O)CC1C(c2ccc(O)cc2)=NOC1c1ccco1] | C19H22N2O4 | 342.39 |
| 00075-1 | 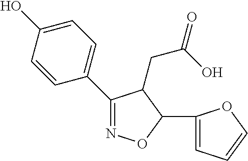 [SMILES: O=C(O)CC1C(c2ccc(O)cc2)=NOC1c1ccco1] | C15H13NO5 | 287.27 |
| 00076-1 | 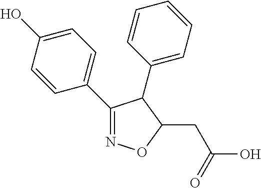 [SMILES: O=C(O)CC1ON=C(c2ccc(O)cc2)C1c1ccccc1] | C17H15NO4 | 297.31 |
| 00077-1 | 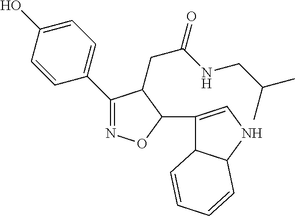 [SMILES: CC(C)CNC(=O)CC1C(c2ccc(O)cc2)=NOC1C1=CNC2C=CC=CC12] | C23H25N3O3 | 391.46 |
| 00080-1 | 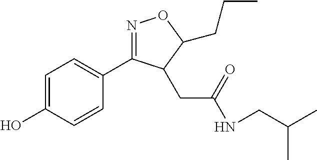 [SMILES: CCCC1ON=C(c2ccc(O)cc2)C1CC(=O)NCC(C)C] | C18H28N2O3 | 318.41 |
| 00081-1 | 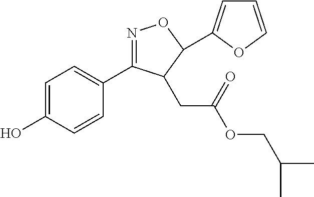 [SMILES: CC(C)COC(=O)CC1C(c2ccc(O)cc2)=NOC1c1ccco1] | C19H21NO5 | 343.37 |
| 00082-1 | 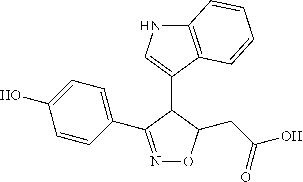 [SMILES: O=C(O)CC1ON=C(c2ccc(O)cc2)C1c1c[nH]c2ccccc12] | C19H16N2O4 | 336.34 |
| 00083-1 | 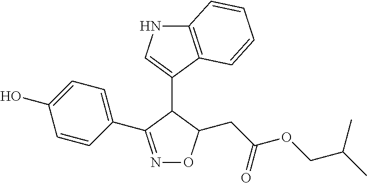 [SMILES: CC(C)COC(=O)CC1ON=C(c2ccc(O)cc2)C1c1c[nH]c2ccccc12] | C23H24N2O4 | 392.45 |
| 00084-1 | 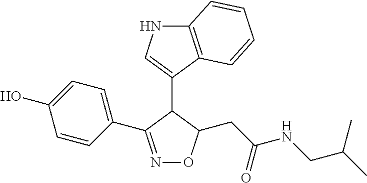 [SMILES: CC(C)CNC(=O)CC1ON=C(c2ccc(O)cc2)C1c1c[nH]c2ccccc12] | C23H25N3O3 | 391.46 |
| 00087-1 | 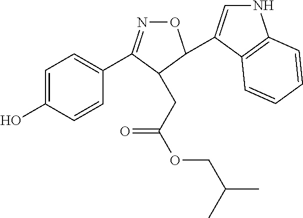 [SMILES: CC(C)COC(=O)CC1C(c2ccc(O)cc2)=NOC1c1c[nH]c2ccccc12] | C23H24N2O4 | 392.45 |
| 00104-1 | 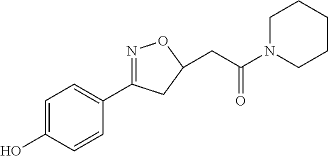 [SMILES: O=C(CC1CC(c2ccc(O)cc2)=NO1)N1CCCCC1] | C16H20N2O3 | 288.34 |
| 00105-1 | 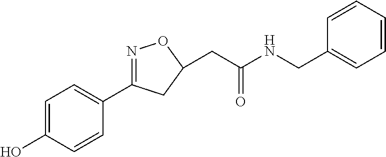 [SMILES: O=C(CC1CC(c2ccc(O)cc2)=NO1)NCc1ccccc1] | C18H18N2O3 | 310.35 |
| 00106-1 | 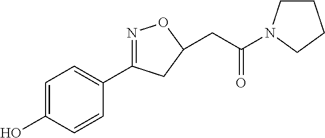 [SMILES: O=C(CC1CC(c2ccc(O)cc2)=NO1)N1CCCC1] | C15H18N2O3 | 274.32 |
| 00107-1 | 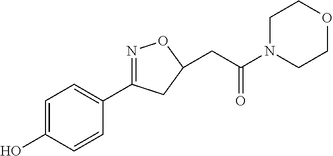 [SMILES: O=C(CC1CC(c2ccc(O)cc2)=NO1)N1CCOCC1] | C15H18N2O4 | 290.31 |
| 00109-1 | 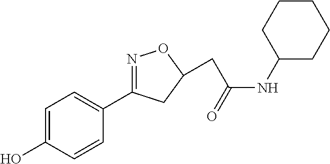 [SMILES: O=C(CC1CC(c2ccc(O)cc2)=NO1)NC1CCCCC1] | C17H22N2O3 | 302.37 |
| 00111-1 | 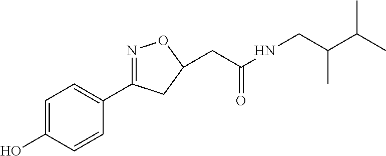 [SMILES: CC(C)C(C)CNC(=O)CC1CC(c2ccc(O)cc2)=NO1] | C17H24N2O3 | 304.38 |
| 00112-1 | 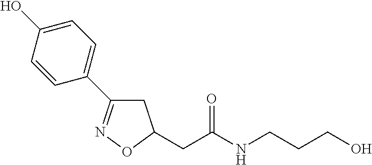 [SMILES: O=C(CC1CC(c2ccc(O)cc2)=NO1)NCCCO] | C14H18N2O4 | 278.3 |
| 00113-1 | 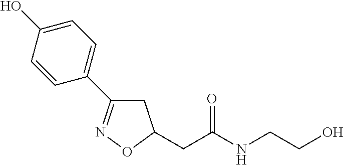 [SMILES: O=C(CC1CC(c2ccc(O)cc2)=NO1)NCCO] | C13H16N2O4 | 264.28 |
| 00114-1 | 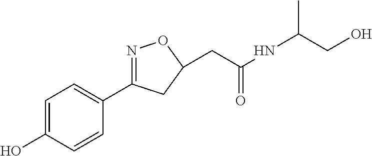 [SMILES: CC(CO)NC(=O)CC1CC(c2ccc(O)cc2)=NO1] | C14H18N2O4 | 278.3 |
| 00123-1 | 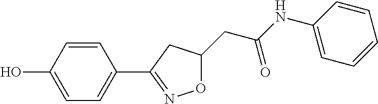 [SMILES: O=C(CC1CC(c2ccc(O)cc2)=NO1)Nc1ccccc1] | C17H16N2O3 | 296.32 |
| 00130-1 | 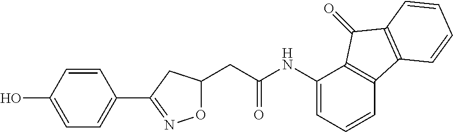 [SMILES: O=C(CC1CC(c2ccc(O)cc2)=NO1)Nc1cccc2c1C(=O)c1ccccc1-2] | C24H18N2O4 | 398.41 |
| 00131-1 | 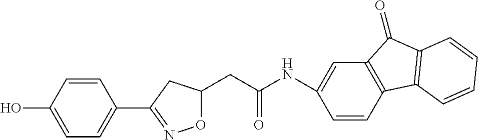 [SMILES: O=C(CC1CC(c2ccc(O)cc2)=NO1)Nc1ccc2c(c1)C(=O)c1ccccc1-2] | C24H18N2O4 | 398.41 |
| 00132-1 | 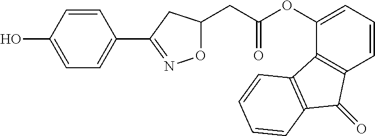 [SMILES: O=C(CC1CC(c2ccc(O)cc2)=NO1)Oc1cccc2c1-c1ccccc1C2=O] | C24H18N2O4 | 398.41 |
| 00133-1 | 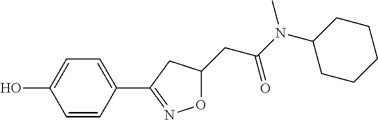 [SMILES: CN(C(=O)CC1CC(c2ccc(O)cc2)=NO1)C1CCCCC1] | C18H24N2O3 | 316.39 |
| 00134-1 | 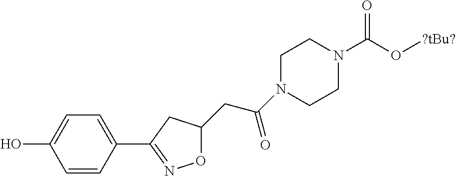 | C16H18N3O5 | 332.33 |
| 00135-1 | 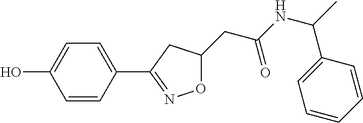 [SMILES: CC(NC(=O)CC1CC(c2ccc(O)cc2)=NO1)c1ccccc1] | C19H20N2O3 | 324.37 |
| 00136-1 | 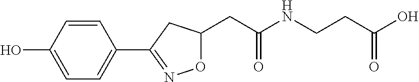 [SMILES: O=C(O)CCNC(=O)CC1CC(c2ccc(O)cc2)=NO1] | C14H16N2O5 | 292.29 |
| 00137-1 | 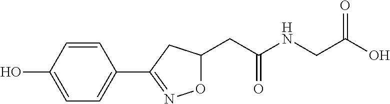 [SMILES: O=C(O)CNC(=O)CC1CC(c2ccc(O)cc2)=NO1] | C13H14N2O5 | 278.26 |
| 00141-1 | 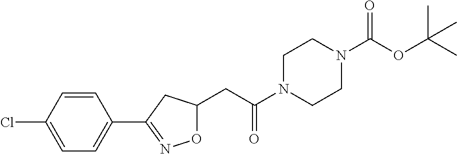 [SMILES: CC(C)(C)OC(=O)N1CCN(C(=O)CC2CC(c3ccc(Cl)cc3)=NO2)CC1] | C20H26N3O4Cl | 407.89 |
| 00143-1 | 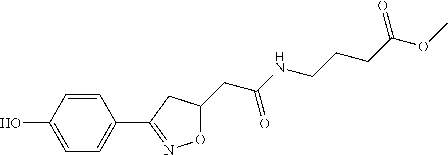 [SMILES: COC(=O)CCCNC(=O)CC1CC(c2ccc(O)cc2)=NO1] | C16H21N2O5 | 320.34 |
| 00144-1 | 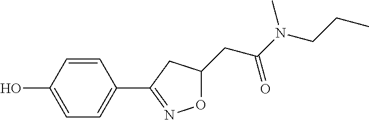 [SMILES: CCCN(C)C(=O)CC1CC(c2ccc(O)cc2)=NO1] | C15H21N2O3 | 276.35 |
| 00148-1 | 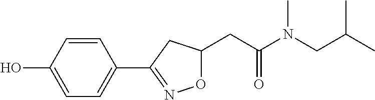 [SMILES: CC(C)CN(C)C(=O)CC1CC(c2ccc(O)cc2)=NO1] | C16H21N2O3 | 290.36 |
| 00149-1 | 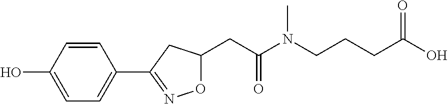 [SMILES: CN(CCCC(=O)O)C(=O)CC1CC(c2ccc(O)cc2)=NO1] | C16H20N2O5 | 320.34 |
| 00150-1 | 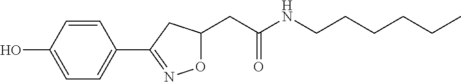 [SMILES: CCCCCCNC(=O)CC1CC(c2ccc(O)cc2)=NO1] | C18H26N2O3 | 304.38 |
| 00151-1 | 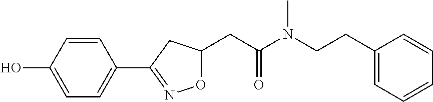 [SMILES: CN(CCc1ccccc1)C(=O)CC1CC(c2ccc(O)cc2)=NO1] | C20H22N2O3 | 338.4 |
| 00152-1 | 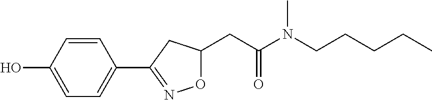 [SMILES: CCCCCN(C)C(=O)CC1CC(c2ccc(O)cc2)=NO1] | C17H24N2O3 | 304.38 |
| 00153-1 | 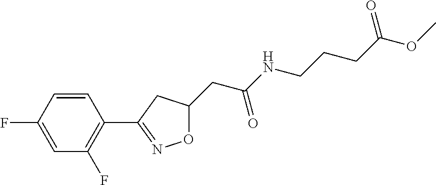 [SMILES: COC(=O)CCCNC(=O)CC1CC(c2ccc(F)cc2F)=NO1] | C16H18N2O4F2 | 340.32 |
| 00154-1 | 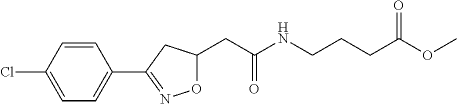 [SMILES: COC(=O)CCCNC(=O)CC1CC(c2ccc(Cl)cc2)=NO1] | C16H19N2O4Cl | 338.79 |
| 00155-1 | 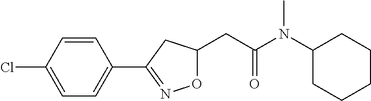 [SMILES: CN(C(=O)CC1CC(c2ccc(Cl)cc2)=NO1)C1CCCCC1] | C18H26N2O2Cl | 334.87 |
| 00156-1 | 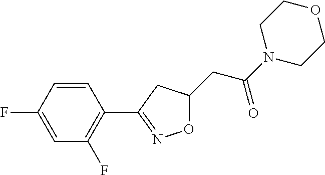 [SMILES: O=C(CC1CC(c2ccc(F)cc2F)=NO1)N1CCOCC1] | C15H16N2O3F2 | 310.28 |
| 00157-1 | 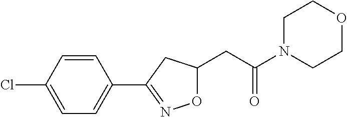 [SMILES: O=C(CC1CC(c2ccc(Cl)cc2)=NO1)N1CCOCC1] | C15H17N2O3Cl | 308.76 |
| 00170-1 | 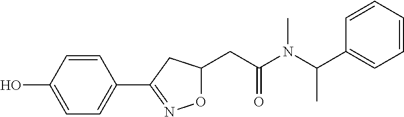 | C26H22N2O3 | 338.4 |
| 00171-1 | 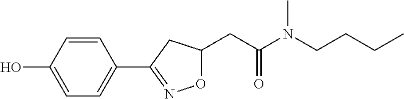 [SMILES: CCCCN(C)C(=O)CC1CC(c2ccc(O)cc2)=NO1] | C16H23N2O3 | 291.36 |
| 00172-1 | 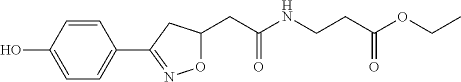 [SMILES: CCOC(=O)CCNC(=O)CC1CC(c2ccc(O)cc2)=NO1] | C16H19N2O5 | 320.34 |
| 00173-1 | 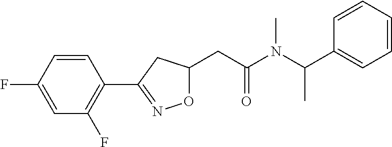 [SMILES: CC(c1ccccc1)N(C)C(=O)CC1CC(c2ccc(F)cc2F)=NO1] | C20H20N2O2F2 | 358.38 |
| 00174-1 | 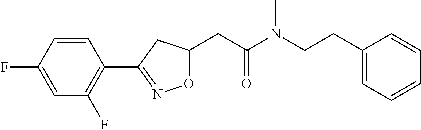 [SMILES: CN(CCc1ccccc1)C(=O)CC1CC(c2ccc(F)cc2F)=NO1] | C20H20N2O2F2 | 358.38 |
| 00175-1 | 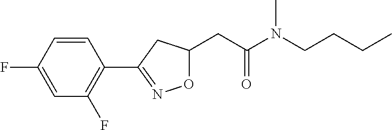 [SMILES: CCCCN(C)C(=O)CC1CC(c2ccc(F)cc2F)=NO1] | C16H20N2O2F2 | 310.34 |
| 00176-1 | 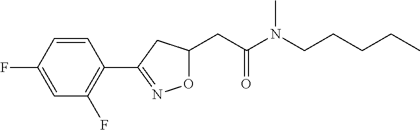 [SMILES: CCCCCN(C)C(=O)CC1CC(c2ccc(F)cc2F)=NO1] | C17H22N2O2F2 | 324.37 |
| 00177-1 | 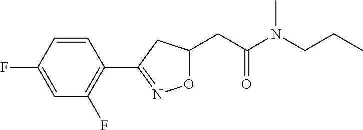 [SMILES: CCCN(C)C(=O)CC1CC(c2ccc(F)cc2F)=NO1] | C15H18N2O2F2 | 296.31 |
| 00178-1 | 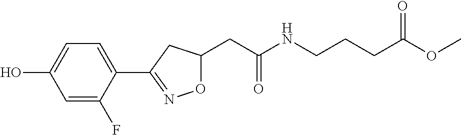 [SMILES: COC(=O)CCCNC(=O)CC1CC(c2ccc(O)cc2F)=NO1] | C19H18N2O5F | 338.33 |
| 00179-1 | 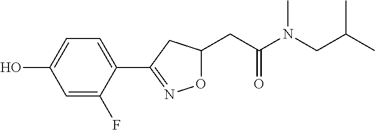 [SMILES: CC(C)CN(C)C(=O)CC1CC(c2ccc(O)cc2F)=NO1] | C16H21N2O3F | 308.35 |
| 00180-1 | 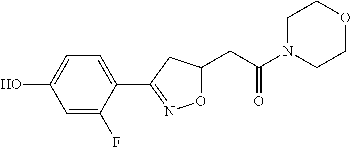 [SMILES: O=C(CC1CC(c2ccc(O)cc2F)=NO1)N1CCOCC1] | C15H17N2O4F | 308.3 |
| 00181-1 | 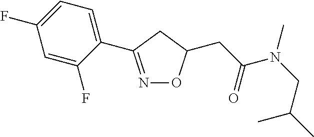 [SMILES: CC(C)CN(C)C(=O)CC1CC(c2ccc(F)cc2F)=NO1] | C16H20N2O2F2 | 310.33 |
| 00182-1 | 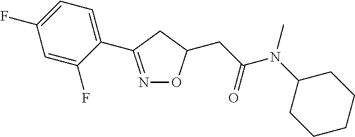 [SMILES: CN(C(=O)CC1CC(c2ccc(F)cc2F)=NO1)C1CCCCC1] | C18H22N2O2F2 | 336.37 |
| 00183-1 | 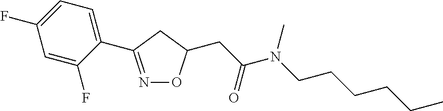 [SMILES: CCCCCCN(C)C(=O)CC1CC(c2ccc(F)cc2F)=NO1] | C18H24N2O2F2 | 338.39 |
| 00184-1 | 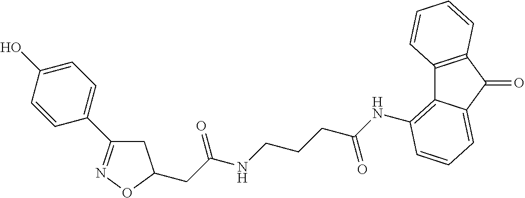 [SMILES: O=C(CC1CC(c2ccc(O)cc2)=NO1)NCCCC(=O)Nc1cccc2c1-c1ccccc1C2=O] | C28H25N3O5 | 483.5 |
| 00185-1 | 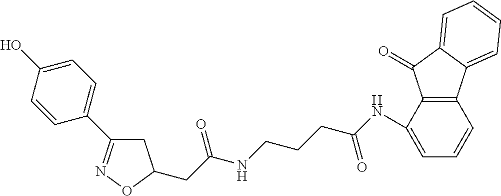 [SMILES: O=C(CC1CC(c2ccc(O)cc2)=NO1)NCCCC(=O)Nc1cccc2c1C(=O)c1ccccc1-2] | C28H25N3O5 | 483.5 |
| 00186-1 | 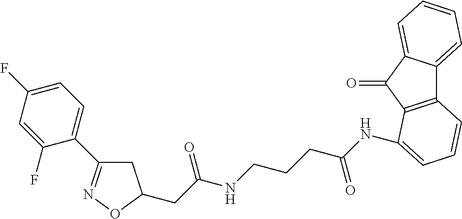 [SMILES: O=C(CC1CC(c2ccc(F)cc2F)=NO1)NCCCC(=O)Nc1cccc2c1C(=O)c1ccccc1-2] | C28H23N3O4F2 | 503.3 |
| 00187-1 | 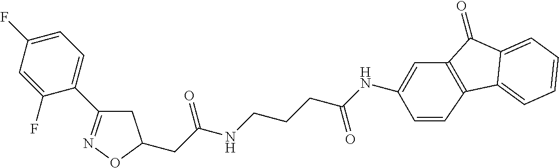 [SMILES: O=C(CC1CC(c2ccc(F)cc2F)=NO1)NCCCC(=O)Nc1ccc2c(c1)C(=O)c1ccccc1-2] | C28H23N3O4F2 | 503.3 |
| 00190-1 | 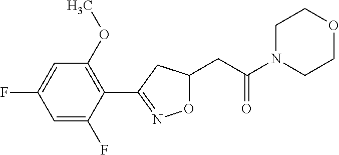 [SMILES: COc1cc(F)cc(F)c1C1=NOC(CC(=O)N2CCOCC2)C1] | C16H18N2O4F2 | 340.3 |
| 00191-1 | 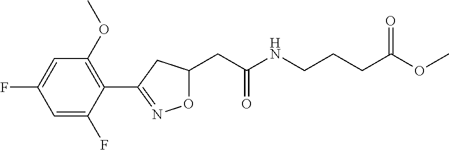 [SMILES: COC(=O)CCCNC(=O)CC1CC(c2c(F)cc(F)cc2OC)=NO1] | C17H20N2O5F2 | 370.35 |
| 00192-1 | 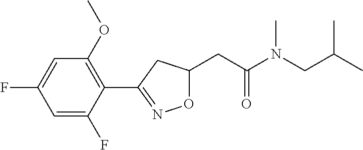 [SMILES: COc1cc(F)cc(F)c1C1=NOC(CC(=O)N(C)CC(C)C)C1] | C17H22N2O3F2 | 340.36 |
| 00193-1 | 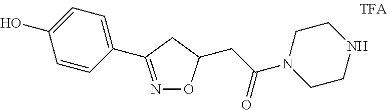 [SMILES: O=C(CC1CC(c2ccc(O)cc2)=NO1)N1CCNCC1] | C14H19O3N3•TFA | 403.35 |
| 00195-1 | 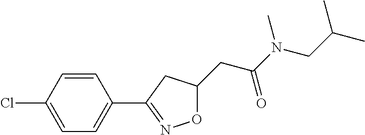 [SMILES: CC(C)CN(C)C(=O)CC1CC(c2ccc(Cl)cc2)=NO1] | C16H21N2O2Cl | 308.80 |
| 00196-1 | 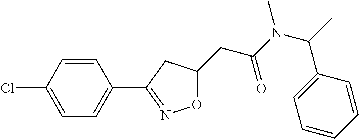 [SMILES: CC(c1ccccc1)N(C)C(=O)CC1CC(c2ccc(Cl)cc2)=NO1] | C20H21N2O2C1 | 356.85 |
| 00197-1 | 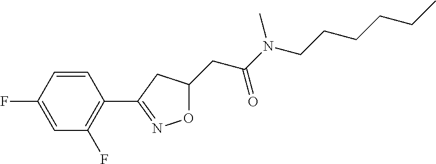 [SMILES: CCCCCCN(C)C(=O)CC1CC(c2ccc(F)cc2F)=NO1] | C18H24F2N2O2 | 338.39 |
| 00199-1 | 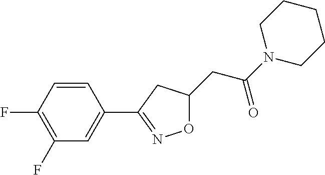 [SMILES: O=C(CC1CC(c2ccc(F)c(F)c2)=NO1)N1CCCCC1] | C16H18F2N2O2 | 308.32 |
| 00202-1 | 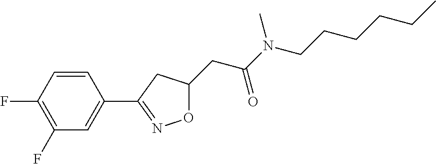 [SMILES: CCCCCCN(C)C(=O)CC1CC(c2ccc(F)c(F)c2)=NO1] | C18H24F2N2O2 | 338.39 |
| 00203-1 | 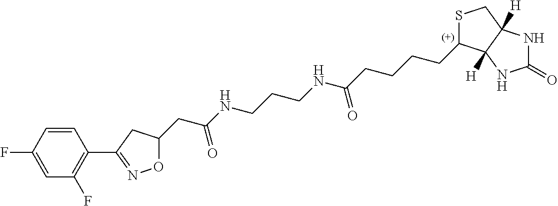 [SMILES: O=C(CCCCC1SC[C@@H]2NC(=O)N[C@H]12)NCCCNC(=O)CC1CC(c2ccc(F)cc2F)=NO1] | C24H31F2N5O4S | 523.6 |
| 00205-1 | 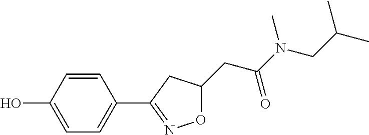 [SMILES: CC(C)CN(C)C(=O)CC1CC(c2ccc(O)cc2)=NO1] | C16H22N2O3 | 290.36 |
| 00206-1 | 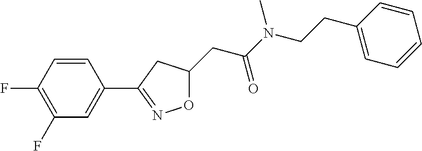 [SMILES: CN(CCc1ccccc1)C(=O)CC1CC(c2ccc(F)c(F)c2)=NO1] | C20H20F2N2O2 | 358.38 |
| 00207-1 | 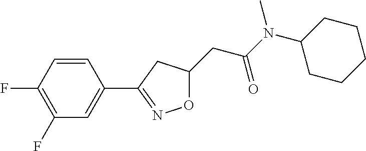 [SMILES: CN(C(=O)CC1CC(c2ccc(F)c(F)c2)=NO1)C1CCCCC1] | C18H22F2N2O2 | 336.38 |
| 00208-1 | 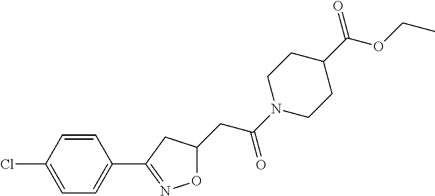 [SMILES: CCOC(=O)C1CCN(C(=O)CC2CC(c3ccc(Cl)cc3)=NO2)CC1] | C19H23ClN2O4 | 378.85 |
| 00254 | 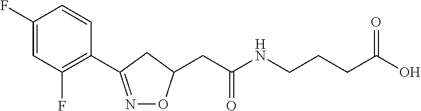 [SMILES: O=C(O)CCCNC(=O)CC1CC(c2ccc(F)cc2F)=NO1] | C15H16F2N2O4 | 326.30 |
| 00274 | 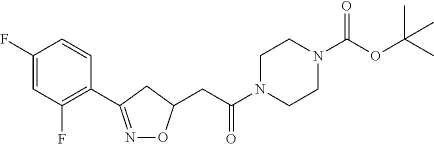 [SMILES: CC(C)(C)OC(=O)N1CCN(C(=O)CC2CC(c3ccc(F)cc3F)=NO2)CC1] | C20H25F2N3O4 | 409.4 |
| 00275 | 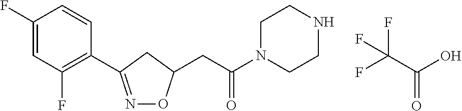 | C15H17F2N3O2•TFA | 423.33 |
| 00294 | 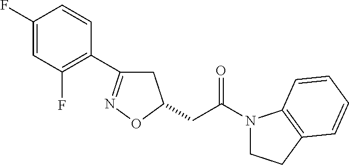 [SMILES: O=C(C[C@H]1CC(c2ccc(F)cc2F)=NO1)N1CCc2ccccc21] | C19H16F2N2O2 | 342.34 |
| 00295 | 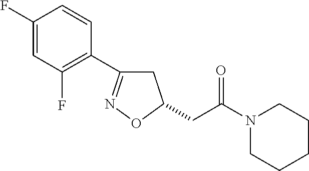 [SMILES: O=C(C[C@H]1CC(c2ccc(F)cc2F)=NO1)N1CCCCC1] | C16H18F2N2O2 | 308.32 |
| 00296 | 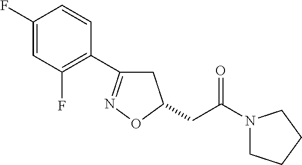 [SMILES: O=C(C[C@H]1CC(c2ccc(F)cc2F)=NO1)N1CCCC1] | C15H16F2N2O2 | 294.3 |
| 00297 | 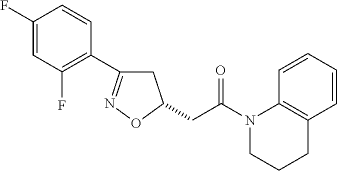 [SMILES: O=C(C[C@H]1CC(c2ccc(F)cc2F)=NO1)N1CCCc2ccccc21] | C20H18F2N2O2 | 356.37 |
| 00298 | 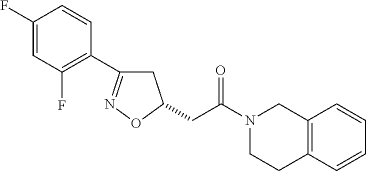 [SMILES: O=C(C[C@H]1CC(c2ccc(F)cc2F)=NO1)N1CCc2ccccc2C1] | C20H18F2N2O2 | 356.37 |
| 00300 | 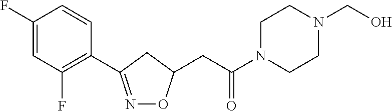 [SMILES: O=C(CC1CC(c2ccc(F)cc2F)=NO1)N1CCN(CO)CC1] | C16H19F2N3O3 | 339.34 |
| 00301 | 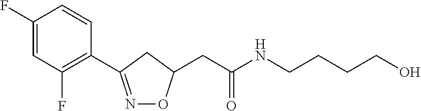 [SMILES: O=C(CC1CC(c2ccc(F)cc2F)=NO1)NCCCCO] | C15H18F2N2O3 | 312.31 |
| 00302 | 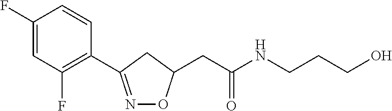 [SMILES: O=C(CC1CC(c2ccc(F)cc2F)=NO1)NCCCO] | C14H16F2N2O3 | 298.29 |
| 00303 | 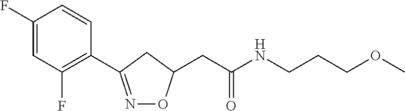 [SMILES: COCCCNC(=O)CC1CC(c2ccc(F)cc2F)=NO1] | C15H18F2N2O3 | 312.31 |
| 00304 | 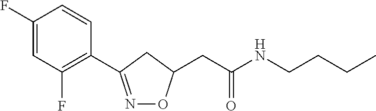 [SMILES: CCCCNC(=O)CC1CC(c2ccc(F)cc2F)=NO1] | C15H18F2N2O2 | 296.31 |
| 00314 | 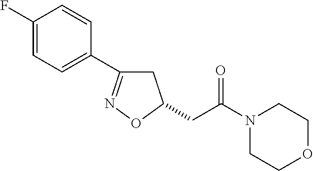 [SMILES: O=C(C[C@H]1CC(c2ccc(F)cc2)=NO1)N1CCOCC1] | C15H17FN2O3 | 292.31 |
| 00315 | 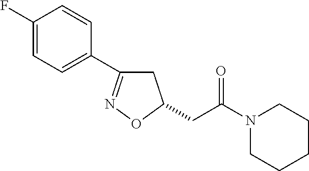 [SMILES: O=C(C[C@H]1CC(c2ccc(F)cc2)=NO1)N1CCCCC1] | C16H19FN2O2 | 290.33 |
| 00316 | 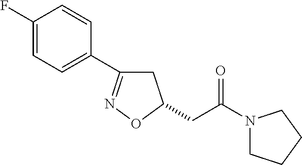 [SMILES: O=C(C[C@H]1CC(c2ccc(F)cc2)=NO1)N1CCCC1] | C15H17FN2O2 | 276.31 |
| 00317 | 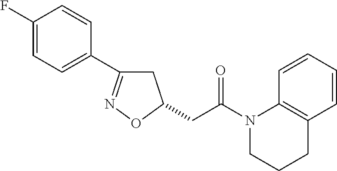 [SMILES: O=C(C[C@H]1CC(c2ccc(F)cc2)=NO1)N1CCCc2ccccc21] | C20H19FN2O2 | 338.38 |
| 00333 | 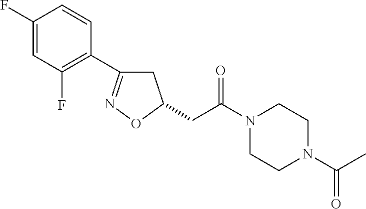 [SMILES: CC(=O)N1CCN(C(=O)C[C@H]2CC(c3ccc(F)cc3F)=NO2)CC1] | C17H19F2N3O3 | 351.35 |
| 00334 | 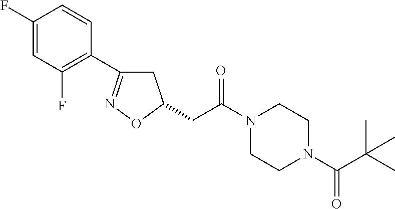 [SMILES: CC(C)(C)C(=O)N1CCN(C(=O)C[C@H]2CC(c3ccc(F)cc3F)=NO2)CC1] | C20H25F2N3O3 | 393.43 |
| 00335 | 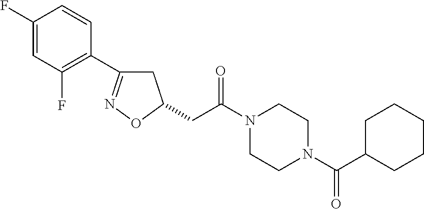 [SMILES: O=C(C[C@H]1CC(c2ccc(F)cc2F)=NO1)N1CCN(C(=O)C2CCCCC2)CC1] | C22H27F2N3O3 | 419.46 |
| 00336 | 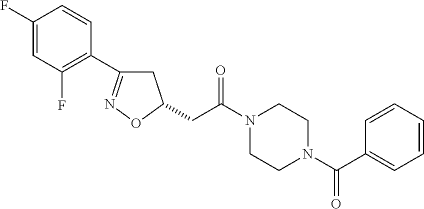 [SMILES: O=C(C[C@H]1CC(c2ccc(F)cc2F)=NO1)N1CCN(C(=O)c2ccccc2)CC1] | C22H21F2N3O3 | 413.42 |
| 00337 | 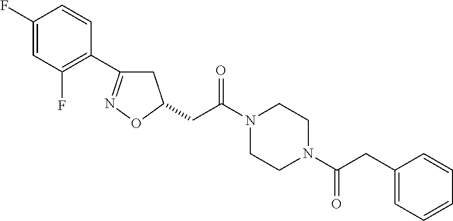 [SMILES: O=C(Cc1ccccc1)N1CCN(C(=O)C[C@H]2CC(c3ccc(F)cc3F)=NO2)CC1] | C23H23F2N3O3 | 427.44 |
| 00338 | 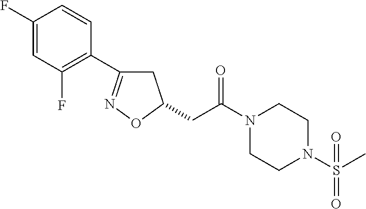 [SMILES: CS(=O)(=O)N1CCN(C(=O)C[C@H]2CC(c3ccc(F)cc3F)=NO2)CC1] | C16H19F2N3O4S | 387.40 |
| 00339 | 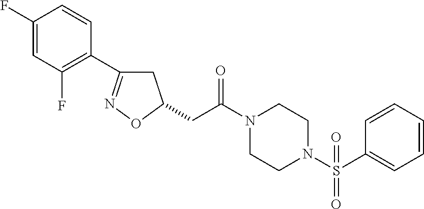 [SMILES: O=C(C[C@H]1CC(c2ccc(F)cc2F)=NO1)N1CCN(S(=O)(=O)c2ccccc2)CC1] | C21H21F2N3O4S | 449.47 |

### EXAMPLES

A further understanding can be obtained by reference to certain specific examples, which are provided herein for purposes of illustration only and are not intended to be limiting unless otherwise specified.

### Example 1

### Synthesis

1. Preparation of 2,4-difluorobenzaldehyde oxime: 2,4-Difluorobenzaldehyde (29.8 g, 209.6 mmol) was dissolved in methanol (500 mL), hydroxylamine hydrochloride (6.8 g, 230.7 mmol) and sodium acetate (34.4 g, 419.4 mmol) were added and the resulting mixture was heated to reflux for 16 hours. The solvent from the crude reaction mixture was partially evaporated in vacuo to give a white slush. The slush was partitioned between water and ethyl acetate. The aqueous layer was extracted twice with ethyl acetate. The combined organic extracts were washed with brine, dried with magnesium sulfate, filtered and evaporated to give a white solid. The solid was redissolved in a 1:2 mixture of warm ethyl acetate and hexanes. The resulting solution was cooled to −10° C. to initiate precipitation. The crystalline material (27.1 g, 91%) was isolated by filtration. ¹H NMR (CDCl₃): δ 8.31 (1H, s), 7.77 (1H, m), 7.47 (1H, s), 6.91 (1H, m), 6.86 (1H, m). m/z (ES⁺) 158.0 ((M+H)⁺, 100%).

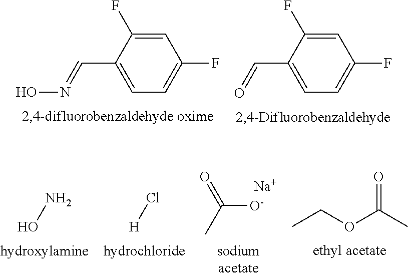

2. Preparation of 2,4-difluoro-N-hydroxybenzimidoyl chloride: 2,4-Difluorobenzaldehyde oxime (5.495 g, 35.35 mmol) was dissolved in dichloromethane (70 mL) and the flask placed in an ice bath. N-Chlorosuccinimide (5.64 g, 42.0 mmol) and dimethylformamide (0.3 mL) were added. The reaction was allowed to warm slowly over a 16 hour period. The reaction was partitioned between water and dichloromethane. The aqueous layer was extracted twice with dichloromethane. The combined organic extracts were dried with magnesium sulfate, filtered and evaporated to give a off-white solid. The solid was redissolved in a 1:2 mixture of dichloromethane and hexanes. The resulting solution was cooled to −10° C. to initiate precipitation. The crystalline material (5.14 g, 76%) was isolated by filtration. ¹H NMR (CDCl₃): δ 8.64 (1H, s), 7.67 (1H, m), 6.94 (2H, m). m/z (ES⁺) 192.0 ((M+H)⁺, 100%).

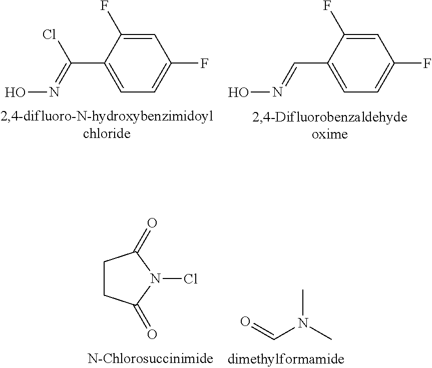

3. Preparation of (1R,2S)-2-phenylcyclohexyl but-3-enoate: (1R,2S)-2-Phenylcyclohexanol (1.17 g, 6.64 mmol), but-3-enoic acid (1.17 g, 19.86 mmol) and p-toluenesulfonic acid monohydrate (0.06 g, 0.33 mmol) were dissolved in toluene (20 mL). The reaction mixture was heated to reflux with a Dean-Stark trap to collect water. After 3 hours, the reaction mixture was diluted with ethyl acetate, washed with saturated, aqueous sodium bicarbonate and water, dried with magnesium sulfate, filtered and the solvent evaporated in vacuo to give the desired product (1.52 g, 94%). ¹H NMR (CDCl₃): δ 7.25 (2H, m), 7.17 (3H, m), 5.58 (1H, m), 4.80 (3H, m), 2.77 (2H, m), 2.66 (1H, m), 2.22 (1H, m), 1.90 (3H, m), 1.47 (4H, m). m/z (ES⁺) 159.2 ((M-C₄H₆O₂+H)⁺, 100%), 245.2 ((M+H)⁺, 7%).

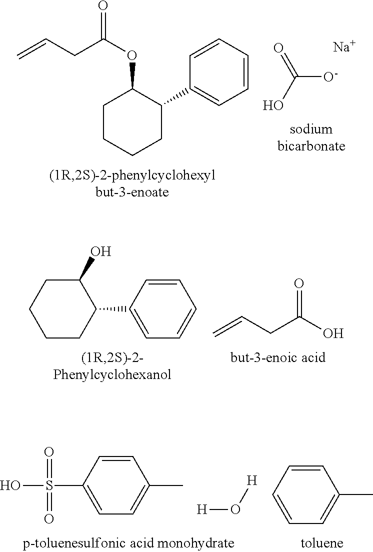

4. Preparation of (1R,2S)-2-phenylcyclohexyl 2-((R)-3-(2,4-difluorophenyl)-4,5-dihydroisoxazol-5-yl)acetate: 2,4-Difluoro-N-hydroxybenzimidoyl chloride (0.2231 g, 1.165 mmol) was dissolved in isopropanol (9 mL) and triethylamine (0.2358 g, 2.330 mmol) was added. (1R,2S)-2-Phenylcyclohexyl but-3-enoate (0.2561 g, 1.048 mmol) was dissolved in isopropanol (10 mL) then added via a syringe pump over a 24 hour period. Dilute hydrochloric acid (1M, 10 mL) and ethyl acetate (10 mL) were added and the layers separated. The aqueous solution was extracted twice with ethyl acetate. The combined organic extracts were dried with magnesium sulfate, filtered and the solvent evaporated in vacuo. The crude solid (0.455 g) has an RSR:RSS ratio of 80:20. This crude material could be hydrolyzed without further purification. To obtain a pure sample of each isomer, the mixture can be recrystallized from hexane or separated by column chromatography on silica gel, eluting with Et₃N/EtOAc/hexanes (1:2:97) ¹H NMR (major isomer in CDCl₃): δ 7.73 (1H, m), 7.28 (2H, m), 7.19 (3H, m), 6.87 (2H, m), 5.03 (1H, m), 4.61 (1H, m), 2.83 (1H, ddd, J₁=18 Hz, J₂=10 Hz, J₃=2 Hz), 2.70 (2H, m), 2.64 (1H, dd, J₁=16 Hz, J₂=7 Hz), 2.33 (1H, dd, J₁=16 Hz, J₂=10 Hz), 2.13 (m, 1H), 1.91 (2H, m), 1.70 (1H, m), 1.47 (4H, m). m/z (ES⁺) 400.2 ((M+H)⁺, 100%).

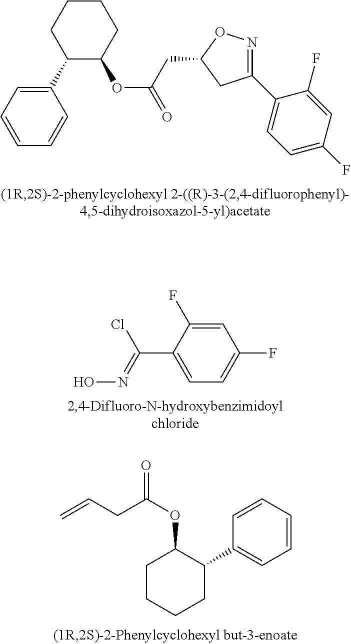 [SMILES: O=C(C[C@H]1CC(c2ccc(F)cc2F)=NO1)O[C@@H]1CCCC[C@H]1c1ccccc1 ; O/N=C(\Cl)c1ccc(F)cc1F ; C=CCC(=O)O[C@@H]1CCCC[C@H]1c1ccccc1]

5. Preparation of (R)-2-(3-(2,4-difluorophenyl)-4,5-dihydroisoxazol-5-yl)acetic acid: (1R,2S)-2-Phenylcyclohexyl 2-((R)-3-(2,4-difluorophenyl)-4,5-dihydroisoxazol-5-yl)acetate (3.20 g, 8.02 mmol) and p-toluenesulfonic acid (0.015 g, 0.0802 mmol) were dissolved in tetrahydrofuran (40 mL) and water (20 mL). The solution was heated to reflux for 13 days. The solvent was partially evaporated in vacuo. The resulting mixture was partitioned between ethyl acetate and water. The organic extract was washed with saturated, aqueous sodium bicarbonate. The bicarbonate solution was acidified with dilute hydrochloric acid (1M) to pH 3. The solution was extracted with ethyl acetate. The combined organic extracts were dried with magnesium sulfate, filtered and the solvent evaporated in vacuo to give the desired product (1.72 g, 89%). ¹H NMR (CDCl₃): δ 7.87 (1H, dt, J₁=8.6 Hz, J₂=6.4 Hz), 6.94 (1H, m), 6.88 (1H, m), 5.13 (1H, m), 3.63 (1H, ddd, J₁=17.5 Hz, J₂=10.5 Hz, J₃=2.5 Hz), 3.20 (1H, ddd, J₁=17.5 Hz, J₂=7.4 Hz, J₃=2.6 Hz), 2.92 (1H, dd, J₁=16.4 Hz, J₂=6.3 Hz), 2.72 (1H, dd, J₁=16.4 Hz, J₂=7.0 Hz). m/z (ES⁺) 242.0 ((M+H)⁺, 100%).

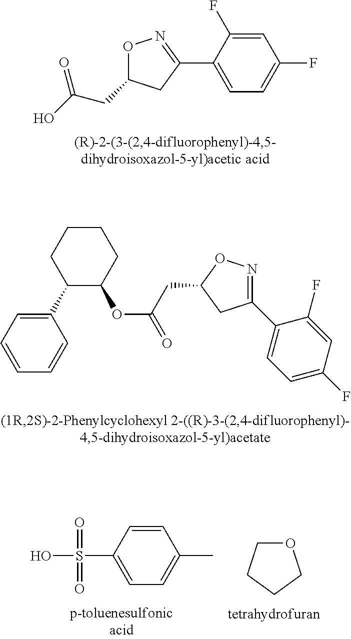

6. Preparation of (R)-2-(3-(2,4-difluorophenyl)-4,5-dihydroisoxazol-5-yl)-1-morpholinoethanone: (R)-2-(3-(2,4-difluorophenyl)-4,5-dihydroisoxazol-5-yl)acetic acid (2.26 g, 9.37 mmol) was dissolved in dichloromethane (15 mL). Oxalyl chloride (1.31 g, 10.31 mmol) was added followed by dimethylformamide (0.05 mL). After 1 hour, the solvent was evaporated in vacuo. The crude acid chloride was redissolved in dichloromethane (15 mL). Morpholine (0.85 g, 9.84 mmol) and triethylamine (0.996 g, 9.84 mmol) were added and the reaction allowed to stir for 16 hours. The reaction mixture was diluted with dichloromethane, washed with dilute hydrochloric acid (1M), dried with magnesium sulfate, filtered and the solvent evaporated. The crude amide was purified by column chromatography on silica gel, eluting with EtOAc/hexanes (20:80) to give a white solid (2.65 g, 91%). ¹H NMR (CDCl₃): δ 7.83 (1H, dt, J₁=8.6 Hz, J₂=6.4 Hz), 6.93 (1H, m), 6.87 (1H, m), 5.13 (1H, m), 3.68 (6H, m), 3.63 (1H, m), 3.49 (2H, m), 3.23 (1H, ddd, J₁=17.7 Hz, J₂=7.5 Hz, J₃=2.6 Hz), 2.93 (1H, dd, J₁=15.6 Hz, J₂=5.3 Hz), 2.64 (1H, dd, J₁=15.6 Hz, J₂=7.7 Hz). m/z (ES⁺) 311.1 ((M+H)⁺, 100%).

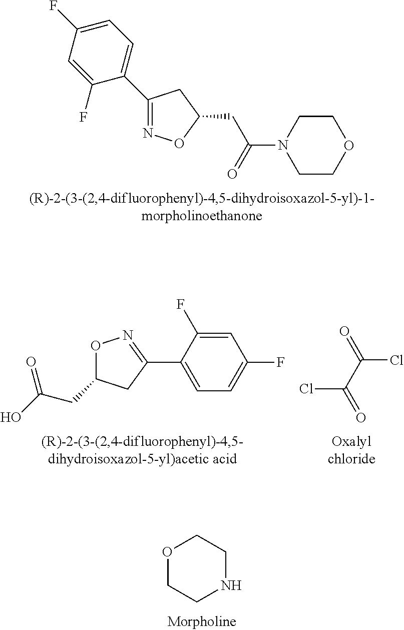

### Example 2

### Chloro Synthesis

1. Preparation of 4-chlorobenzaldehyde oxime: 4-Chlorobenzaldehyde (10.0 g, 71.2 mmol) was dissolved in methanol (125 mL), hydroxylamine hydrochloride (5.94 g, 85.4 mmol) and sodium acetate (14.58 g, 177.8 mmol) were added and the resulting mixture was heated to reflux for 16 hours. The solvent from the crude reaction mixture was partially evaporated in vacuo to give a white slush. The slush was partitioned between water and ethyl acetate. The aqueous layer was extracted twice with ethyl acetate. The combined organic extracts were washed with brine, dried with magnesium sulfate, filtered and evaporated to give a white solid. The solid was redissolved in a 1:2 mixture of warm ethyl acetate and hexanes. The resulting solution was cooled to −10° C. to initiate precipitation. The crystalline material (8.83 g, 72%) was isolated by filtration. ¹H NMR (CDCl₃): δ 8.11 (1H, s), 7.80 (1H, bs), 7.51 (2H, d, J=8.6 Hz), 7.36 (2H, d, J=8.4 Hz). m/z (ES⁺) 156.0 ((M+H)⁺, 100%).

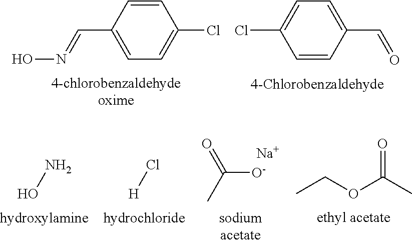

2. Preparation of 4-chloro-N-hydroxybenzimidoyl chloride: 4-Chlorobenzaldehyde oxime (7.33 g, 47.1 mmol) was dissolved in dimethylformamide (10 mL) and the flask placed in an ice bath. N-Chlorosuccinimide (6.29 g, 47.1 mmol) dissolved in dimethylformamide (15 mL) was added. The reaction was allowed to warm slowly over a 16 hour period. The reaction was partitioned between water and ethyl acetate. The aqueous layer was extracted twice with ethyl acetate. The combined organic extracts were dried with magnesium sulfate, filtered and evaporated to give a off-white solid. The solid was redissolved in a 1:2 mixture of ethyl acetate and hexanes. The resulting solution was cooled to −10° C. to initiate precipitation. The crystalline material (6.20 g, 76%) was isolated by filtration. ¹H NMR (CDCl₃): δ 7.98 (1H, bs), 7.80 (2H, d, J=8.8 Hz), 7.40 (2H, d, J=9.0 Hz). m/z (ES⁺) 190.0 ((M+H)⁺, 100%)

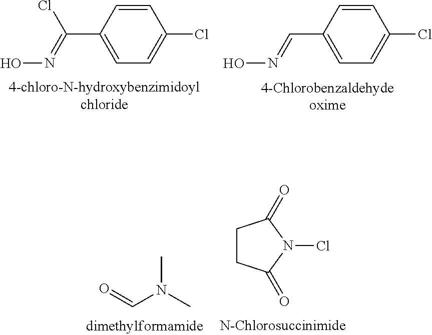

3. Preparation of (1R,2S)-2-phenylcyclohexyl 2-((R)-3-(4-chlorophenyl)-4,5-dihydroisoxazol-5-yl)acetate: 4-Chloro-N-hydroxybenzimidoyl chloride (0.291 g, 1.53 mmol) was dissolved in dichloromethane (10 mL) and triethylamine (0.229 g, 2.26 mmol) was added. (1R,2S)-2-Phenylcyclohexyl but-3-enoate (0.275 g, 1.13 mmol) was dissolved in dichloromethane (10 mL) then added via a syringe pump over a 24 hour period. Dilute hydrochloric acid (1M, 10 mL) and ethyl acetate (10 mL) were added and the layers separated. The aqueous solution was extracted twice with ethyl acetate. The combined organic extracts were dried with magnesium sulfate, filtered and the solvent evaporated in vacuo. The crude solid (0.455 g) has an RSR:RSS ratio of 95:5. This crude material could be hydrolyzed without further purification. To obtain a pure sample of each isomer, the mixture can be recrystallized from hexane or separated by column chromatography on silica gel, eluting with Et₃N/EtOAc/hexanes (1:2:97) ¹H NMR (major isomer in CDCl₃): δ 8.16 (1H, d, J=8.7 Hz), 8.11 (1H, d, J=8.7 Hz), 7.55 (1H, d, J=8.7 Hz), 7.50 (1H, d, J=8.7 Hz), 7.25 (2H, m), 7.18 (3H, m), 5.58 (1H, m), 4.97 (2H, m), 2.79 (2H, m), 2.66 (1H, m), 2.12 (1H, m), 1.86 (m, 3H), 1.57 (1H, m), 1.47 (4H, m). m/z (ES⁺) 398.2 ((M+H)⁺, 100%).

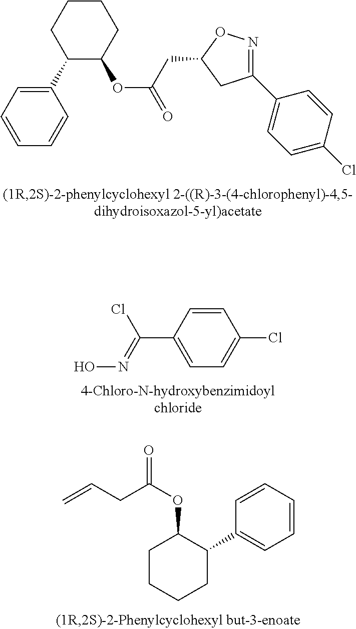 [SMILES: O=C(C[C@H]1CC(c2ccc(Cl)cc2)=NO1)O[C@@H]1CCCC[C@H]1c1ccccc1 ; O/N=C(\Cl)c1ccc(Cl)cc1 ; C=CCC(=O)O[C@@H]1CCCC[C@H]1c1ccccc1]

### Example 3

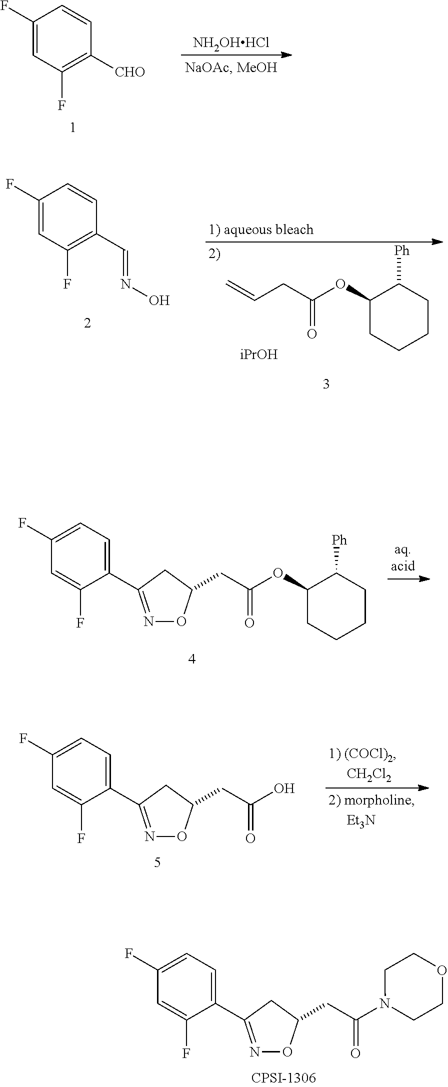

### Example 4

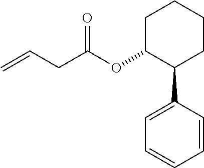

1. Preparation of (1R,2S)-2-phenylcyclohexyl but-3-enoate: (1R,2S)-trans-2-phenyl-1-cyclohexanol (2.15 g, 12.2 mmol), 3-butenoic acid (3.21 g, 37.3 mmol) and 4-toluenesulfonic acid (0.11 g, 0.59 mmol) were dissolved in toluene (30 mL). The reaction was stirred at reflux for 16 hours with azeotropic removal of water via a Dean-Stark trap. The solution was washed with aqueous, saturated sodium bicarbonate and then with water. The organic layer was dried with MgSO₄ and the solvent evaporated to give 2.65 g (89%) of the desired product: ¹H NMR δ 7.25 (m, 2H), 7.18 (m, 3H), 5.59 (m, 1H), 4.95 (m, 3H), 2.79 (m, 2H), 2.66 (m, 1H), 2.12 (d, 1H, J=10.2 Hz), 1.85 (m, 3H), 1.45 (m, 4H).

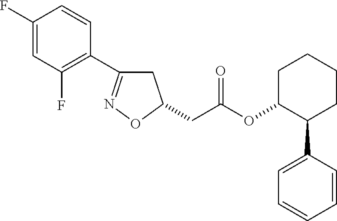

2. Preparation of (1R,2S)-2-phenylcyclohexyl 2-((R)-3-(2,4-difluorophenyl)-4,5-dihydroisoxazol-5-yl)acetate: 2,4-difluoro-N-hydroxybenzimidoyl chloride (0.105 g, 0.669 mmol) was dissolved in isopropanol (10 mL). N,N-Diisopropylethyl amine (2.87 g, 22.3 mmol) was added and the mixture heated to 60° C. (1R,2S)-2-phenylcyclohexyl but-3-enoate (0.100 g, 0.445 mmol) was dissolved in isopropanol (6 mL) and slowly added to the other solution via syringe pump over a 6 hour period and then the reaction was stirred for an additional 8 hours. The reaction was partitioned between ethyl acetate and 1.0 M aqueous hydrochloric acid. The aqueous layer was extracted with ethyl acetate. The combined organic extracts were washed with water, dried with MgSO₄ and the solvent evaporated. Chiral HPLC analysis shows a greater than 99.5:0.5 diastereomeric ratio. The crude product was purified by silica gel chromatography, eluting with 10% ethyl acetate in hexanes, to give 0.116 g (65%) of the desired product. ¹H NMR δ 7.86 (dt, 1H, J=6.4 Hz, J′=8.6 Hz), 7.25 (m, 2H), 7.18 (m, 3H), 6.90 (m, 2H), 5.13 (m, 1H), 4.95 (m, 1H), 3.59 (ddd, 1H, J=2.6 Hz, J′=10.5 Hz, J″=17.4 Hz), 3.19 (ddd, 1H, J=2.6 Hz, J′=7.4 Hz, J″=17.4 Hz), 2.87 (dd, 1H, J=6.2 Hz, J′=16.0 Hz), 2.79 (m, 2H), 2.66 (m, 2H), 2.12 (d, 1H, J=10.2 Hz), 1.85 (m, 3H), 1.45 (m, 4H); LRMS (ESI) m/z C₁₁H₉F₂NO³ (M+H⁺-cyclohexenylbenzene) 242.1, found 242.1.

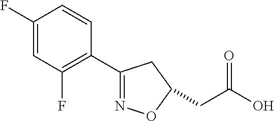 [SMILES: O=C(O)C[C@H]1CC(c2ccc(F)cc2F)=NO1]

3. Preparation of 2(R)-2-(3-(2,4-difluorophenyl)-4,5-dihydroisoxazol-5-yl)acetic acid: (1R,2S)-2-phenylcyclohexyl 2-((R)-3-(2,4-difluorophenyl)-4,5-dihydroisoxazol-5-yl)acetate (7.48 g, 18.7 mmol) was dissolved in trifluoromethanesulfonic acid solution (20 mL) which contains water (2 mL). The reaction was stirred at room temperature for 24 hours. Silica gel (5 g) was added to the crude reaction mixture and the solvent was evaporated in vacuo. Chromatography with silica gel, eluting with 50% ethyl acetate/hexanes solvent system, gives 3.88 g product (86%). ¹H NMR δ 7.86 (dt, 1H, J=6.4 Hz, J′=8.6 Hz), 6.90 (m, 2H), 5.13 (m, 1H), 3.59 (ddd, 1H, J=2.6 Hz, J′=10.5 Hz, J″=17.4 Hz), 3.19 (ddd, 1H, J=2.6 Hz, J′=7.4 Hz, J″=17.4 Hz), 2.87 (dd, 1H, J=6.2 Hz, J′=16.0 Hz), 2.66 (dd, 1H, J=7.3 Hz, J′=16.0 Hz); LRMS (ESI) m/z C₁₁H₁₀F₂NO₃ (M+H⁺) 242.1, found 242.0.

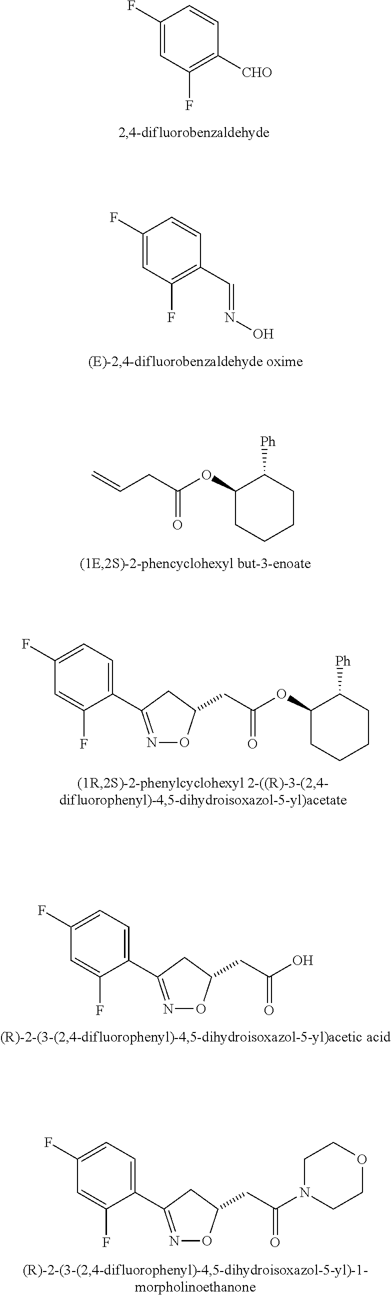 [SMILES: O=Cc1ccc(F)cc1F ; O/N=C/c1ccc(F)cc1F ; C=CCC(=O)O[C@@H]1CCCC[C@H]1c1ccccc1 ; O=C(C[C@H]1CC(c2ccc(F)cc2F)=NO1)O[C@@H]1CCCC[C@H]1c1ccccc1 ; O=C(O)C[C@H]1CC(c2ccc(F)cc2F)=NO1 ; O=C(C[C@H]1CC(c2ccc(F)cc2F)=NO1)N1CCOCC1]

The entire contents of U.S. application Ser. Nos. 10/164,630, filed Jun. 10, 2002; 10/927,494, filed Aug. 27, 2004; and 11/090,128, filed Mar. 28, 2005, are independently incorporated herein by reference.

This application is based on and claims priority to U.S. Provisional Application Ser. No. 61/264,406, filed Nov. 25, 2009, the entire contents of which are hereby incorporated by reference.

1. A process, comprising:
reacting, in the presence of a base, a compound having the following formula (I): 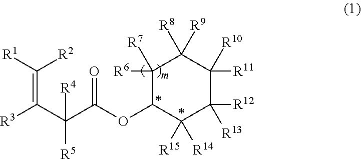
with a compound having the following formula (II): 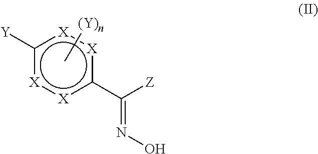
to produce a compound having the following formula (III): 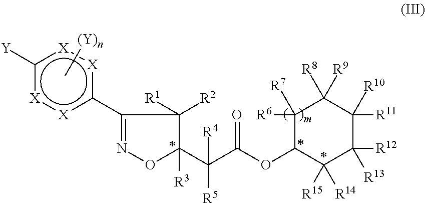
wherein at least the carbons marked “*” are chiral;
wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;
wherein R⁶, R⁷, R⁸, R⁹, R¹⁰, R¹¹, R¹², R¹³, R¹⁴, and R¹⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein m is 0 or 1; wherein any two of R⁶, R⁷, R⁸, R⁹, R¹⁰, R¹¹, R¹², R¹³, R¹⁴, and R¹⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; with the proviso that R¹⁴ and R¹⁵ are not identical groups;
wherein each X is independently carbon or nitrogen, wherein when any X is carbon, it comprises a Y substituent, n being an integer of from 1 to 4 and being the number of X's that are carbon;
wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms; and
wherein Z is iodine, bromine, chlorine, alkoxy group, aryloxy group, alkylcarbonyloxy group, alkoxycarbonyloxy group, alkylsulfonyloxy group, or alkoxysulfonyloxy group, arylcarbonyloxy group, aryloxycarbonyloxy group, arylsulfonyloxy group, or aryloxysulfonyloxy group.

2. The process of claim 1, wherein the compound having the formula (I) is selected from the following compounds:

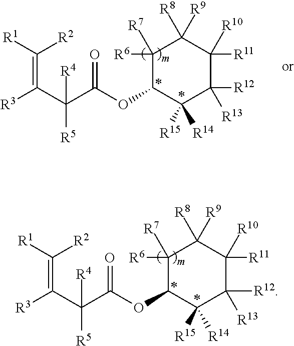

3. The process of claim 1, wherein the compound having the formula (III) is one or more selected from the following compounds:

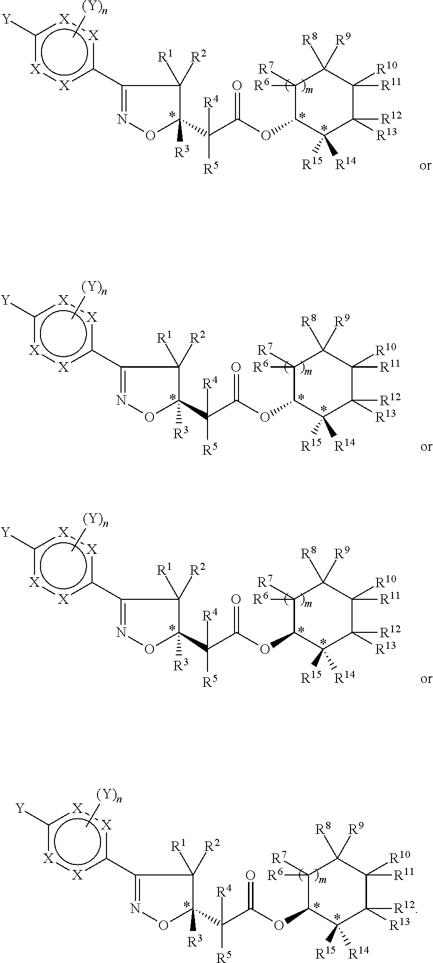

4. The process of claim 1, wherein the compound having the formula (I) is the following compound: 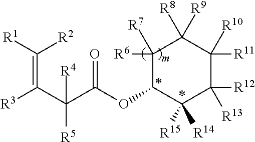
and the compound having the formula (III) is one or more of the following compounds:

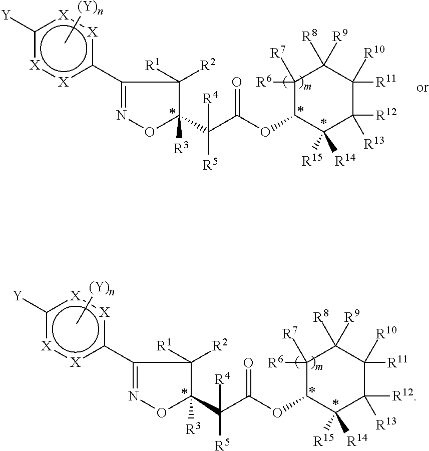

5. The process of claim 4, further comprising separating the compounds having the following formulas from one another:

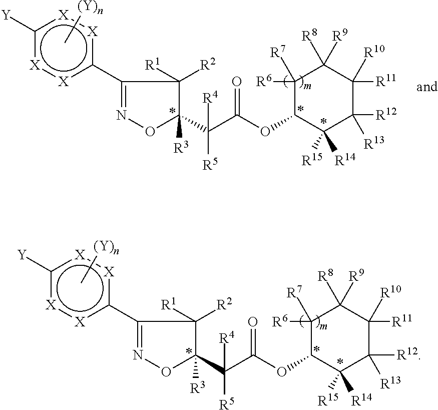

6. The process of claim 5, wherein the separating is carried out by a process comprising recrystallization, chromatography, or a combination thereof.

7. The process of claim 1, wherein the compound having the formula (I) is the following compound: 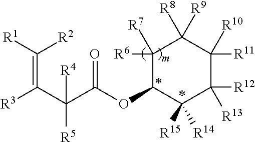
and the compound having the formula (III) is one or more of the following compounds:

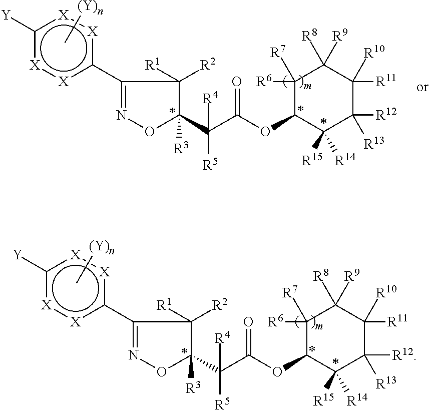

8. The process of claim 7, further comprising separating the compounds having the formulas from one another:

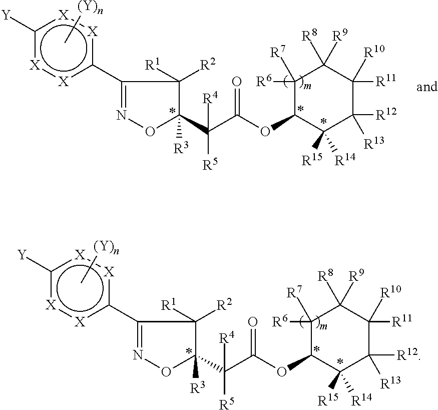

9. The process of claim 8, wherein the separating is carried out by a process comprising recrystallization, chromatography, or a combination thereof.

10. The process of claim 1, wherein the compound having the formula (III) is selected from one of the following compounds:

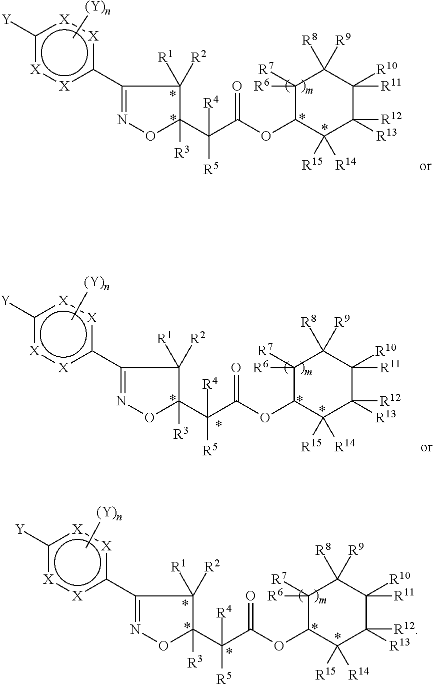

11. The process of claim 1, wherein R¹, R², R³, R⁴, and R⁵ are each independently hydrogen, an alkyl group, a cycloalkyl group, an alkenyl group, an alkynyl group, an oxo group, an aryl group, a heterocyclic group, a heteroaryl group, an aralkyl group, a heteroaralkyl group, an amino group, an alkylamino group, a dialkylamino group, an amidine group, an amide group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms.

12. The process of claim 1, wherein R⁶, R⁷, R⁸, R⁹, R¹⁰, R¹¹, R¹², R¹³, R¹⁴, and R¹⁵ are each independently hydrogen, an alkyl group, a cycloalkyl group, a halo group, an alkenyl group, an alkynyl group, a hydroxy group, an oxo group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein m is 0 or 1; wherein any two of R⁶, R⁷, R⁸, R⁹, R¹⁰, R¹¹, R¹², R¹³, R¹⁴, and R¹⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; with the proviso that R¹⁴ and R¹⁵ are not identical groups.

13. The process of claim 1, wherein Z is iodine, bromine, or chlorine.

14. The process of claim 1, wherein one or both of R⁴ and R⁵ are hydrogen.

15. The process of claim 1, wherein only one of R⁴ and R⁵ is hydrogen.

16. The process of claim 1, further comprising reacting at least one of the compounds having formula (I), (III), or a combination thereof, in which one or both of R⁴ and R⁵ are hydrogen, with an electrophile in the presence of a base, to independently and optionally selectively replace one or both of the hydrogens at R⁴ and R⁵ with at least one selected from the group including an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein each group may be optionally and independently straight or branched; wherein each group may be optionally and independently substituted by one or more independent substituents; and wherein one or more than one atom in each group may be optionally and independently replaced with one or more independent heteroatoms.

17. The process of claim 1, further comprising contacting at least one of the compounds having formula (I), (III), or a combination thereof, in which only one of R⁴ and R⁵ is hydrogen, with a base in the absence of an electrophile, and allowing to equilibrate, to produce a thermodynamically favored equilibration product.

18-33. (canceled)

34. The process of claim 1, further comprising reacting the compound having formula (III): 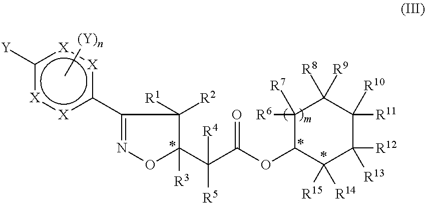
with an acid;
to produce a compound having the following formula (IV): 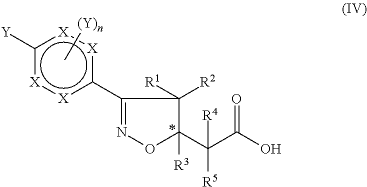
wherein at least the carbons marked “*” are chiral;
wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;
wherein R⁶, R⁷, R⁸, R⁹, R¹⁰, R¹¹, R¹², R¹³, R¹⁴, and R¹⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein m is 0 or 1; wherein any two of R⁶, R⁷, R⁸, R⁹, R¹⁰, R¹¹, R¹², R¹³, R¹⁴, and R¹⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms; with the proviso that R¹⁴ and R¹⁵ are not identical groups;
wherein each X is independently carbon or nitrogen, wherein when any X is carbon, it comprises a Y substituent, n being an integer of from 1 to 4 and being the number of X's that are carbon; and
wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms.

35-44. (canceled)

45. The process of claim 34, further comprising:
reacting, in the presence of an acid halide and simultaneously or subsequently at least one selected from a base, an inorganic base, a trialkylamine, an aromatic nitrogen-containing heterocycle, or an organometallic compound, the compound having formula (IV): 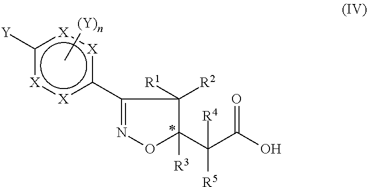
with a compound having at least one nucleophilic O, N, S, C, B or combination thereof, which forms a new bond with an electrophile;
to produce a compound having the following formula (V): 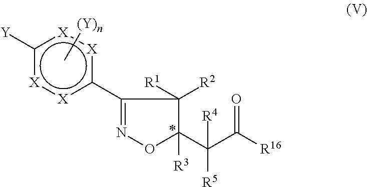
wherein at least the carbons marked “*” are chiral;
wherein R¹, R², R³, R⁴, and R⁵ are each independently hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein R¹ and R² may be taken together to form a cyclic group; wherein R⁴ and R⁵ may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms;
wherein each X is independently carbon or nitrogen, wherein when any X is carbon, it comprises a Y substituent, n being an integer of from 1 to 4 and being the number of X's that are carbon;
wherein each Y is independently a carbonyl group, a carboxylic acid group, a carboxylate group, hydrogen, an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein two Y groups may be taken together to form a cyclic or aryl group; wherein each group may be optionally and independently straight or branched; wherein each group may be optionally and independently substituted by one or more independent substituents; and wherein one or more than one atom in each group may be optionally and independently replaced with one or more independent heteroatoms;
and wherein R¹⁶ is an alkyl group, a cycloalkyl group, a halo group, an alkenyl group, an alkynyl group, a hydroxy group, an oxo group, a mercapto group, an alkylthio group, an alkoxy group, an aryl group, a heterocyclic group, a heteroaryl group, an aryloxy group, a heteroaryloxy group, an aralkyl group, a heteroaralkyl group, an aralkoxy group, a heteroaralkoxy group, an amino group, an alkylamino group, a dialkylamino group, an amidine group, an amide group, a carbamoyl group, an alkylcarbonyl group, an alkoxycarbonyl group, an alkylaminocarbonyl group, a dialkylamino carbonyl group, an arylcarbonyl group, an aryloxycarbonyl group, an alkylsulfonyl group, an arylsulfonyl group, perhaloalkyl group, a perhaloalkoxy group, a perhalocycloalkyl group, a perhaloalkenyl group, a perhaloalkynyl group, a perhaloaryl group, or a perhaloaralkyl group; wherein any two alkyl groups may be taken together to form a cyclic group; wherein each group may be optionally and independently straight or branched; wherein each group may be optionally and independently substituted by one or more independent substituents; and wherein one or more than one atom in each group may be optionally and independently replaced with one or more independent heteroatoms.

46-133. (canceled)

134. The process of claim 45, wherein the compound having formula (V) is selected from the following compounds: 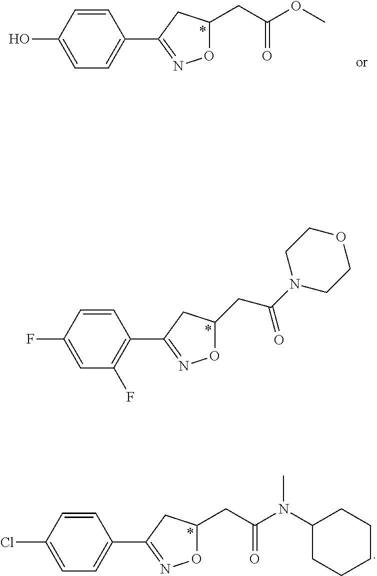 [SMILES: COC(=O)CC1CC(c2ccc(O)cc2)=NO1 ; O=C(CC1CC(c2ccc(F)cc2F)=NO1)N1CCOCC1 ; CN(C(=O)CC1CC(c2ccc(Cl)cc2)=NO1)C1CCCCC1]
